(19) United States
(12) Patent Application Publication
(10) Pub. No.: US 2026/0365596 A1
(43) Pub. Date: Nov. 19, 2026

(54) EFFICIENT AND SELECTIVE UPCYCLING OF POLYETHYLENE TO ALKYLBENZENES UNDER MODERATE HYDROGEN PRESSURE

(71) Applicant: THE REGENTS OF THE UNIVERSITY OF CALIFORNIA, Oakland, CA (US)

(72) Inventors: Jiakai SUN, Santa Barbara, CA (US); Susannah L. SCOTT, Santa Barbara, CA (US); Yu-Hsuan LEE, Santa Barbara, CA (US); Mahdi ABU-OMAR, Santa Barbara, CA (US)

(21) Appl. No.: 19/484,884

(22) PCT Filed: May 16, 2024
(86) PCT No.: PCT/US2024/029667

§ 371 Date: Nov. 17, 2025

Related U.S. Application Data
(60) Provisional application No. 63/502,520, filed on May 16, 2023.

(51) Int. Cl. C07C 4/06; C07C 15/02

(52) U.S. Cl. CPC C07C 4/06; C07C 15/02; C07C 2523/42

(57) ABSTRACT

Process for upcycling a waste material to form alkylbenzene compounds is described herein. The process typically includes the steps of feeding a waste material containing hydrocarbon polymer(s) into a reactor containing a catalyst therein, and operating the reactor at a sufficient temperature, under a sufficient H₂ pressure, and for a sufficient period of time to convert the hydrocarbon polymer(s) to a liquid product containing one or more alkylbenzene compounds. The catalyst contains a transition metal or a mixture of a transition metal and another metal, and may be dispersed on the surface of an acidic support. Using the disclosed process, an alkylbenzene mass selectivity of at least 25% relative to all aromatic compounds in the liquid product, is achieved.

### CROSS-REFERENCE TO RELATED APPLICATION

This application claims the benefit of and priority to U.S. Provisional Application No. 63/502,520 filed May 16, 2023, the entire content of which is incorporated herein by reference for all purpose in its entirety.

### STATEMENT REGARDING FEDERALLY SPONSORED RESEARCH OR DEVELOPMENT

This invention was made with government support under DE-AC02-07°CH11358 awarded by the United States Department of Energy. The government has certain rights in the invention.

### FIELD OF THE INVENTION

The invention is generally in the field of upcycling of hydrocarbon polymers, particularly methods for upcycling of waste hydrocarbon polymers.

### BACKGROUND OF THE INVENTION

Conversion of polyethylene (PE) to surfactant-range alkylaromatics (C₁₆-22) could add value to the used polymer and promote the recycling of waste plastic. The initial report on this process indicates slow reaction kinetics, which limits its practical application, particularly when considering the high rate of plastic production and disposal. Subsequently, Brønsted acid sites (BAS) were shown to complement metal sites by playing important roles in C—C bond scission, isomerization, and aromatization. Bifunctional catalysts accelerate depolymerization to alkylbenzenes, however, too much acidity leads to the undesirable formation of polyaromatics and carbon residue.

Further, industrial production of alkylbenzenes currently uses a high H₂ pressure. For example, naphtha catalytic reforming is conducted under 7-68 atm H₂, where aromatic yields are a maximum. The P(H₂) value is a tradeoff: lower P(H₂) leads to catalyst coking, while higher P(H₂) suppresses aromatic formation. In the context of PE conversion, the rate of C—C bond scission plays a role in achieving the desired molecular weight for alkylbenzenes in an acceptable time. Since alkylbenzene formation from LDPE is a combination of reforming and hydrocracking, the reaction conditions needed to increase the rate of formation and yield of desirable monoaromatic products require a proper P(H₂).

There remains a need for improved processes for upcycling hydrocarbon polymers.

Therefore, it is the object of the present invention to provide a process for upcycling plastic waste containing hydrocarbon polymers to more valuable materials, such as alkylbenzenes.

It is a further object of the present invention to provide an improved depolymerization process to produce more valuable materials, such as alkylbenzenes.

It is a further object of the present invention to provide compositions formed from depolymerization processes.

It is a further object of the present invention to provide compositions formed from upcycling plastic waste.

### SUMMARY OF THE INVENTION

Processes for upcycling plastic waste containing one or more hydrocarbon polymers to alkylbenzenes are described herein. The process disclosed herein is operated under a sufficient hydrogen gas (H₂) pressure, and thereby enhances the yield of alkylbenzenes and the selectivity of alkylbenzenes relative to polyaromatic compounds (which are environmental pollutants) resulting from the catalytic depolymerization of the plastic waste.

The process typically includes (i) feeding a plastic waste containing a hydrocarbon polymer, optionally, more than one hydrocarbon polymer, into a reactor and (ii) operating the reactor at a sufficient temperature, under a sufficient hydrogen gas (H₂) pressure, and for a sufficient period of time to convert the hydrocarbon polymer(s) to a product containing a liquid.

The liquid in the product contains an alkylbenzene compound, optionally more than one alkylbenzene compound. In some forms, the liquid in the product also contains other aromatic compounds, such as one or more alkylpolyaromatic compounds and/or one or more polyaromatic compounds. When other aromatic compounds are present in the liquid, the mass selectivity of the alkylbenzene compound(s) (also referred to herein as alkylbenzene mass selectivity) relative to all aromatic compounds in the liquid is at least 25%, at least 30%, at least 50%, in a range from 25% to 99.99%, from 25% to 99.9%, from 25% to 99%, from 25% to 95%, from 25% to 90%, from 25% to 90%, from 25% to 80%, from 25% to 70%, or from 25% to 60%. Further, the disclosed process can produce the alkylbenzene compound(s) with a yield of at least 4% or at least 5%, by weight.

The reactor contains a catalyst therein. The catalyst includes a transition metal. Typically, the catalyst is in the form of atoms, nanoclusters, or nanoparticles, or a combination thereof. Optionally, the catalyst is dispersed in the form of atoms, nanocluster, or nanoparticles, or a combination thereof on the surface of a support.

Typically, the reactor operates under a H₂ pressure that is effective to achieve a mass selectivity of the alkylbenzene compound(s) of at least 25%, relative to all aromatic compounds in the liquid of the product. For example, the reactor operates under a H₂ pressure in a range from 1 atm to 8 atm, from 1 atm to 7 atm, from 1 atm to 6 atm, from 1 atm to 5 atm, or from 1 atm to 4 atm for depolymerizing a plastic waste contained therein, and the liquid product obtained from the depolymerization reaction contains alkylbenzene compound(s) with a mass selectivity of at least 25% relative to all aromatic compounds in the liquid product. The H₂ pressure under which the reactor operates may be measured immediately prior to the depolymerization reaction in step (ii) or during step (ii). Optionally, P(H₂) is measured continuously or at regular intervals during step (ii) and the desired P(H₂) is maintained during step (ii).

### BRIEF DESCRIPTION OF THE DRAWINGS

FIG. 1a is a graph showing representative GC-FID chromatograms of gas-phase hydrocarbon products and their molar distribution (inset), recovered from the conversion of PE (0.200 g, M_{w}=3.5×10³ g mol⁻¹, Ð=1.9) catalyzed by Pt/SiO₂—Al₂O₃ (0.100 g) conducted for 24 h at 275 °C without addition of external H₂. Propene was added as an internal standard.

FIG. 1b is a bar graph showing the molar selectivity of hydrocarbon products with different carbon numbers in gas.

FIGS. 2a-2o are graphs showing GC-MS ion chromatograms for the liquid-phase hydrocarbon products obtained from the reaction of PE (0.200 g, M_{w}=3.5×10³ g mol⁻¹, Ð=1.9) catalyzed by Pt/SiO₂—Al₂O₃ (0.100 g) after 24 h at 275 °C conducted with 4 bar external H₂ (FIGS. 2a-2e); molar product distributions calculated assuming the MS response factors depending only on the type of hydrocarbon (e.g., alkane vs. alkylbenzene) (FIGS. 2f-2j); and molar product distributions calculated assuming the MS response factors depending on the type of hydrocarbon and depend linearly on the molecular weight (FIGS. 2k-2o), for alkylbenzenes (FIGS. 2a, 2f, 2k), alkylnaphthalenes (FIGS. 2b, 2g, 2l), alkylphenanthrenes (FIGS. 2c, 2h, 2m), alkanes (FIGS. 2d, 2i, 2n), and total hydrocarbons (FIGS. 2e, 2j, 2o).

FIG. 3a is a graph showing distribution of liquid-phase products obtained from the reaction of PE (0.200 g, M_{w}=3.5×10³ g mol⁻¹, Ð=1.9) catalyzed by Pt/SiO₂—Al₂O₃ (0.100 g) after 24 h at 275 °C, conducted in the absence of external H₂. The inset figure is an expansion of the short retention time region, showing assignments for the major peaks. FIG. 3b is a zoom-in view of the data shown in FIG. 3a.

FIG. 4a is a graph showing gel permeation chromatography (GPC) analysis of product distribution (black) and corresponding carbon numbers (red) for products of the conversion of PE (0.200 g, M_{w}=3.5×10³ g mol⁻¹, Ð=1.9) catalyzed by Pt/SiO₂—Al₂O₃ (0.100 g) after 24 h reaction at 275 °C in the absence of external H₂. The inset is an expansion of the low-molecular weight region. FIG. 4b is a zoom-in view of the data shown in FIG. 4a.

FIGS. 5a-5j are graphs showing thermogravimetric analysis (TGA) data (bottom curve) and the corresponding differential data (DTGA, top curve), measured in air, for unused Pt/SiO₂—Al₂O₃(FIG. 5a) unconvertedPE (M_{w}=3.5×10³ g mol⁻¹, Ð=1.9) (FIG. 5b), and the insoluble residue, recovered following CD₂°Cl₂ extraction of soluble hydrocarbons, from the reaction of PE in the absence of external H₂ (FIG. 5c), and in the presence of various amounts of external H₂: 1 bar (FIG. 5d), 2 bar (FIG. 5e), 3 bar (FIG. 5f), 4 bar (FIG. 5g), 5 bar (FIG. 5h), 6 bar (FIG. 5i), and 8 bar (FIG. 5j). Reaction conditions: PE (0.200 g, M_{w}=3.5×10³ g mol−³, Ð=1.9), Pt/SiO₂—Al₂O₃ (0.100 g), 275 °C, 24 h.

FIGS. 6a and 6b are graphs comparing the GPC chromatograms with refractive index detection (RI, rihgt) and ultraviolet detection (UV, left) for the liquid hydrocarbons recovered from the conversion of PE (0.200 g, M_{w}=3.5×10³ g mol⁻¹, Ð=1.9) catalyzed by Pt/SiO₂—Al₂O₃ (0.100 g) for 24 h, in the absence of external H₂ (FIG. 6a), and under 1 bar external H₂ (FIG. 6b).

FIG. 7a is a graph showing the dependence on reactor P(H₂) of the average carbon number for alkylaromatics (red) and total hydrocarbons (blue) in the combined liquid+wax fraction. Reaction conditions: PE (0.200 g, M_{w}=3.5×10³ g mol⁻¹, Ð=1.9), Pt/SiO₂—Al₂O₃ (0.100 g, 1.7 wt %), 24 h, 275 °C. FIG. 7b is a graph showing the dependence on time of the average carbon number of aromatics (red) and total hydrocarbons (blue) in the liquid and wax fraction, obtained from the reaction of PE (M_{w}=3.5×10³ g mol⁻¹, Ð=1.9) catalyzed by Pt/SiO₂—Al₂O₃ (0.100 g) for 0-12 h at 275 °C under P(H₂)=2 bar.

FIGS. 8a-8c are graphs showing the effect of external P(H₂) on the mass fractions of various hydrocarbon types (from top to bottom: gases (C₁₋₆), liquids (C₇₋₃₀) soluble in CH₂°Cl₂, waxes (C_{>30}) soluble in CH₂°Cl₂, organic residues insoluble in CH₂°Cl₂) (FIG. 8a); molecular weight distributions for the wax products, measured by GPC-RI (note that waxes are only formed in appreciable amounts for P(H₂)<6 bar) (FIG. 8b), and the average carbon number in the combined liquid+wax fractions, and the total number of C—C bond scission events (FIG. 8c). Batch reaction conditions: 0.200 g PE (M_{w}=3.5×10³ g mol⁻¹, Ð=1.9), Pt/SiO₂—Al₂O₃ (0.100 g), 275 °C, 24 h.

FIGS. 9a and 9b are graphs showing the effect of external P(H₂) on ¹H NMR spectra, recorded in CD₂Cl₂, of the combined liquid/wax products recovered from the catalytic conversion of PE (FIG. 9a), and the molar yield of alkylbenzenes and the alkylbenzene selectivity relative to all aromatics (FIG. 9b). Batch reaction conditions: 0.200 g PE (M_{w}=3.5×10³ g mol⁻¹, Ð=1.9), Pt/SiO₂—Al₂O₃ (0.100 g), 275 °C, 24 h.

FIGS. 10a and 10b are graphs showing the dependence on reactor P(H₂) of GC-MS of liquid products: intensities of characteristic ion fragments for alkylbenzenes (m/z=91, 92, 105, 106, 119, 120, 133, 134, green), alkyldecalins (m/z=138, 152, 166, 180, 194, blue), alkyltetralins (m/z=132, 146, 160, 174, 188, 202, 216, 230, light blue),¹⁸ alkylnaphthalenes (m/z=115, yellow), and alkylphenanthrenes (m/z=178, 192, 206, 220, 234, 248, red) relative to the total ion counts (FIG. 10a), and intensities of the characteristic ions for polyaromatics (including alkylnaphthalenes and alkylphenanthrenes, yellow) and monoaromatics (alkylbenzenes and alkyldecalins, green) relative to total aromatics (sum of mono- and polyaromatics) (FIG. 10b). Reaction conditions: PE (0.200 g, M_{w}=3.5×10³ g mol⁻¹, Ð=1.9), Pt/SiO₂—Al₂O₃ (0.100 g), 24 h, 275 °C.

FIGS. 11a-11c are graphs showing the effect of changing the reaction atmosphere during batch depolymerization of PE (M_{w}=3.5×10³ g mol⁻¹, Ð=1.9, 0.200 g) catalyzed by Pt/SiO₂—Al₂O₃ (0.100 g) at 275 °C. The reactor was evacuated and filled with the amount of H₂ indicated after each 4 h reaction period (FIG. 11a). FIG. 11b shows the total number of C—C bond scission events. FIG. 11c shows the total yield of alkylbenzenes.

FIG. 12 is a scheme illustrating an exemplary process for catalytic upcycling of plastic waste containing hydrocarbon polymers to alkylbenzene compounds.

### DETAILED DESCRIPTION OF THE INVENTION

### I. Process for Depolymerizing a Waste Solid

Process for upcycling a plastic waste material is described herein. Generally, the term “upcycling” refers to the depolymerization of a waste material to more valuable chemicals, such as alkylbenzene compounds. The waste contains a hydrocarbon polymer, optionally more than one hydrocarbon polymer. In the process, the hydrocarbon polymer in the waste is generally upcycled to form one or more alkylbenzene compounds. The process can also be a process for manufacturing various alkylbenzene compounds.

The upcycling process includes a depolymerization reaction. Optionally, the depolymerization reaction is solvent-free. For example, the waste material is in a solid form and is not dissolved in a solvent prior to or during the depolymerization reaction. The depolymerization reaction is performed under a suitable hydrogen gas (H₂) pressure. Typically, the H₂ pressure is effective to achieve a high mass selectivity (i.e., at least 15%) of alkylbenzene compound(s) relative to all aromatic compounds in the product formed by depolymerization of a plastic waste material.

The process generally includes (i) feeding a waste material containing a hydrocarbon polymer, optionally more than one hydrocarbon polymer, into a reactor, where the reactor contains a catalyst therein; and (ii) operating the reactor under a sufficient H₂ pressure to convert the hydrocarbon polymer(s) to a product. The product contains a liquid (also referred to herein as “liquid product”). The liquid product contains an alkylbenzene compound, optionally more than one alkylbenzene compound. Generally, during step (ii), the reactor is operated at a sufficient temperature, under the sufficient H₂ pressure, for a sufficient period of time, to form the product.

During step (ii), the H₂ pressure in the reactor can be a constant H₂ pressure for the duration of the reaction, or varied H₂ pressures, such as two or more different H₂ pressures, over the duration of the reaction. In some forms, the reactor is operated under a constant H₂ pressure to form the product. When a constant H₂ pressure is applied, hydrogen gas may be fed into the reactor during step (ii), one or more times, to maintain the constant P(H₂). In these instances, P(H₂) can be measured continuously or at regular intervals during the depolymerization reaction such that the desired P(H₂) is maintained during the reaction. For example, the P(H₂) in the reactor is maintained during the reaction in step (ii) at a value ranging from 1 atm to 8 atm, from 1 atm to 7 atm, from 1 atm to 6 atm, from 1 atm to 5 atm, or from 1 atm to 4 atm, such as 8 atm.

In some forms, the reactor is operated under two different H₂ pressures to produce the product. For example, step (ii) of the process includes step (iia) operating the reactor under a first P(H₂) to form fragments of the hydrocarbon polymer, and after step (iia), step (iib) operating the reactor under a second P(H₂) to form the product. Typically, the fragments of the hydrocarbon polymer formed in step (iia) are saturated fragments. Typically, the second P(H₂) is lower than the first P(H₂). For example, the first P(H₂) ranges from 5 atm to 20 atm, from 5 atm to 18 atm, from 5 atm to 15 atm, from 5 atm to 12 atm, from 5 atm to 10 atm, such as about 8 atm, and/or the second P(H₂) ranges from 0 to <5 atm, from 0 to 4 atm, from 0 to 3 atm, or from 0 to 2 atm. When the P(H₂) in the reactor is varied from a first P(H₂) to a second P(H₂) that is lower than the first P(H₂), the process can further include in step (iib) removing hydrogen gas from the reactor to provide the second P(H₂). In these forms, when the reactor is operated under the first P(H₂), the plastic waste material contained therein is depolymerized to form fragments of the hydrocarbon polymer having low weight average molecular weight (Mw). Typically, in these forms, the fragments of the hydrocarbon polymers are measured continuously or at regular intervals during step (iia) under the first P(H₂) to determine the weight average molecular weight (Mw) of the fragments. Methods for measuring the fragments of the hydrocarbon polymers to determine Mw during step (iia) are known. For example, a sample of the hydrocarbon polymer fragments is obtained from the reactor continuously or periodically and subject to measurement and analysis using a suitable instrument, such as NMR, Mass-spectrometry, etc. Generally, when the fragments of the hydrocarbon polymer obtained under the first P(H₂) reach an average chain length of <100 carbon atoms, the P(H₂) in the reactor is lowered to the second P(H₂), such as by removing the hydrogen gas from the reactor. When the reactor is operated under the second P(H₂), the liquid product that contains alkylbenzene compound(s) is formed. In some forms, the second P(H₂) can be 0. In these forms, the reactor in step (iib) is operated under a pressure of an inert gas, such as argon or nitrogen gas. The inert gas pressure can range from 0 to <5 atm, from 0 to 4 atm, from 0 to 3 atm, or from 0 to 2 atm. When an inert gas is used to provide a pressure in the reactor, the process includes, in step (iib), removing hydrogen gas from the reactor and feeding the inert gas into the reactor to provide the inert gas pressure.

In some forms, the reactor is operated under three different H₂ pressures to produce the product. For example, step (ii) of the process includes step (iia) operating the reactor under a first P(H₂) to form fragments of the hydrocarbon polymer, after step (iia), step (iib) operating the reactor under a second P(H₂) to form aromatic compounds, and after step (iib), step (iic) operating the reactor at a third P(H₂) to form the product. Typically, the second P(H₂) is lower than the first P(H₂), and the third P(H₂) is higher than the second P(H₂). For example, the first P(H₂) and second P(H₂) each are in a range as described above; and the third P(H₂) ranges from 5 atm to 15 atm, from 5 atm to 12 atm, or from 5 atm to 10 atm. The reaction under the first P(H₂), methods for determining the reaction stage for pressure change, and methods for adjusting H₂ pressure from the first P(H₂) to the second P(H₂) and optionally providing an inert gas pressure in step (iib) are as described above. When the reactor is operated under the second P(H₂), aromatic compounds that contain alkylbenzenes, polyaromatics, etc. are formed. Typically, in step (iib), the aromatic compounds are measured continuously or at regular intervals to determine the total yield of the aromatic compounds. Methods for measuring the aromatic compounds to determine their total yield during step (iib) are known. For example, a sample of the aromatic compounds is obtained from the reactor continuously or periodically and subject to measurement and analysis using a suitable instrument, such as ¹H NMR spectroscopy combined with gas chromatography. More specific methods for measuring and analyzing a sample of the aromatic compounds to determine their total yield are described in the Examples below. Generally, when the total yield of the aromatic compounds obtained under the second P(H₂) reaches a plateau, the P(H₂) in the reactor is increased to provide the third P(H₂) in step (iic), such as by feeding hydrogen gas into the reactor. When an inert gas is used in step (iib) to provide an inert gas pressure, the inert gas can be removed from the reactor using any suitable method and hydrogen gas can be fed into the reactor simultaneously with or subsequently to the inert gas removal process. In some forms, the flow of hydrogen gas into the reactor can replace any inert gas remaining in the reactor and thereby remove the inert gas from the reactor. In some forms, even an inert gas is used in step (iib) to provide an inert gas pressure, in step (iic) after step (iib), the reactor can be pressurized with H₂ without removing the inert gas from the reactor. When the reactor is operated under the third P(H₂) in step (iic), polyaromatics in the aromatic compounds formed in step (iib) can be converted to alkylbenzenes and thereby increase the yield of alkylbenzenes and remove undesired polyaromatics in the product.

Optionally, the liquid product also contains other aromatic compounds that are not alkylbenzene compounds, such as one or more alkylpolyaromatic compounds and/or one or more polyaromatic compounds. Typically, the alkylpolyaromatic compounds and the polyaromatic compounds in the liquid product contain from 7 to 40 carbon atoms. Typically, the mass selectivity of the alkylbenzene compound(s), relative to all aromatic compounds in the liquid product, is at least 25%, at least 30%, at least 50%, in a range from 25% to 99.99%, from 25% to 99.9%, from 25% to 99%, from 25% to 95%, from 25% to 90%, from 25% to 80%, from 25% to 70%, or from 25% to 60%. When alkylbenzene compound(s) is/are the only aromatic compound(s) contained in the liquid product, the mass selectivity of the alkylbenzene compound(s) is 100%. The improved alkylbenzene mass selectivity is desirable because other aromatic compounds, such as fused-ring aromatics, in the liquid product formed by depolymerization of a plastic waste material are environmental pollutants with toxic, mutagenic and/or carcinogenic properties.

Optionally, the disclosed process further includes: (a) feeding hydrogen gas into the reactor during step (i) or after step (i) and prior to step (ii). After the hydrogen gas is fed into the reactor, the H₂ pressure desired for the depolymerization reaction in step (ii) is achieved in the reactor.

The inclusion of a H₂ pressure in the process disclosed herein for performing the depolymerization of a plastic waste enhances the yield of alkylbenzenes and the selectivity of alkylbenzenes relative to all aromatic compounds resulting from the catalytic depolymerization of the plastic waste. Without being bound by theory, it is believed that the addition of H₂ pressure in the reactor can promote C—C bond scission and result in the production of hydrocarbon fragments. In contrast to existing industrial processes that show a negative reaction order in H₂ for small molecule hydrocracking, the pseudo-zeroth-order rate of C—C bond scission (r_{C-C scission}) for the depolymerization reaction in the disclosed process shows a positive reaction order in H₂.

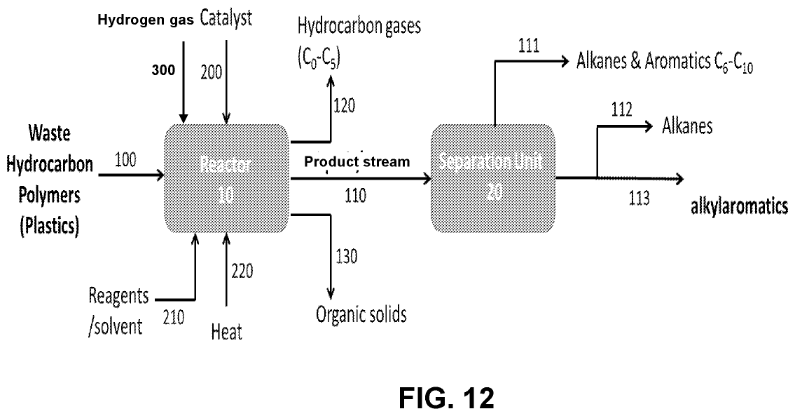

An exemplary process for upcycling a waste material is illustrated in FIG. 12 and described below.

A. Feed a Waste Material and Hydrogen Gas into a Reactor

Generally, the waste material is fed into the reactor. Optionally, when the waste material is fed into the reactor, the reactor is under an inert gas environment, such as argon gas. The inert gas is typically removed from the reactor before feeding hydrogen gas into the reactor to achieve a sufficient H₂ pressure for performing the depolymerization reaction in step (ii).

Optionally, the waste material is in the form of a solid. The waste material contains a plastic, optionally a mixed plastic. Examples of suitable plastics that can be upcycled using the process include, but are not limited to, non-chlorinated plastics, such as polyolefins, polyethylene, polypropylene, polystyrene, and copolymers thereof; and chlorinated plastics, such as polyvinylchloride (PVC), polyvinylidene chloride (PVDC), etc.

In the reactor, the waste material is in contact with the catalyst in the reactor. Reactors that can be used to upcycle a plastic waste material are known. For example, the reactor can be an autoclave reactor, such as a pressure reactor system, a flow reactor, a fixed-bed reactor, a packed-bed reactor (PBR), a continuous stirred tank reactor (CSTR), or a semi-batch reactor.

### 1. Hydrocarbon Polymers

The waste material contains a hydrocarbon polymer. The hydrocarbon polymer in the waste can be polyethylene, polypropylene, polystyrene, a copolymer of polyethylene, a copolymer of polypropylene, a copolymer of polyethylene and polypropylene, or acrylonitrile butadiene styrene (ABS). For example, the waste material contains polyethylene or polypropylene. Optionally, the waste material contains more than one hydrocarbon polymer described above, i.e., a mixture of two or more hydrocarbon polymers described above. For example, the waste material contains a mixture of polyethylene and polypropylene.

Optionally, the waste material contains a hydrocarbon polymer, which is a mixture of different densities of the same hydrocarbon polymer. For example, the waste material can contain polyethylene, which is a mixture of high density polyethylene (HDPE) and low density polyethylene (LDPE).

LDPE generally has a density in the range of 917 to 930 kg/m³. It is not reactive at room temperatures, except by strong oxidizing agents, and some solvents cause swelling. LDPE has more branching (on about 2% of the carbon atoms) than HDPE.

The density of HDPE generally is in the range of 930 to 970 kg/m³. Although the density of HDPE is only marginally higher than that of low-density polyethylene, HDPE has little branching, giving it stronger intermolecular forces and tensile strength than LDPE. The difference in strength between HDPE and LDPE exceeds the difference in density, giving HDPE a higher specific strength (a material's strength (force per unit area at failure) divided by its density; also known as the strength-to-weight ratio).

Optionally, the waste material contains a hydrocarbon polymer of different molecular weights (weight average molecular weight Mw or number average molecular weight Mn). For example, the waste material contains polyethylene, which is a mixture of high molecular weight polyethylene and medium molecular weight polyethylene, a mixture of high molecular weight polyethylene and low molecular weight polyethylene, a mixture of medium molecular weight polyethylene and low molecular weight polyethylene, or a mixture of high, medium, and low molecular weight polyethylene. Generally, a hydrocarbon polymer of low Mw is in the range from 1000 g mol⁻¹ to 10,000 g mol⁻¹; a hydrocarbon polymer of medium Mw is in the range from 10,000 g mol⁻¹ to 200,000 g mol⁻¹; a hydrocarbon polymer of high Mw is in the range from 200,000 g mol⁻¹ to 500,000 g mol⁻¹; and a hydrocarbon polymer of ultrahigh Mw is in the range from 500,000 g mol⁻¹ to 7,500,000 g mol⁻¹.

a. Average Molecular Weight

Typically, the hydrocarbon polymer in the waste material has a weight average molecular weight (Mw) of at least 1×10³ g mol⁻¹, 2×10³ g mol⁻¹, at least 2.5×10³ g mol⁻¹, at least 3×10³ g mol⁻¹, at least 3.5×10³ g mol⁻¹, at least 4×10³ g mol⁻¹, up to 7.5×10⁶ g mol⁻¹, 5×10⁶ g mol⁻¹, 1×10⁶ g mol⁻¹, 5×10⁵ g mol⁻¹, up to 1×10⁵ g mol⁻¹, up to 9.5×10⁴ g mol⁻¹, up to 9×10⁴ g mol⁻¹, up to 8.5×10⁴ g mol⁻¹, up to 8×10⁴ g mol⁻¹, up to 7.5×10⁴ g mol⁻¹, up to 7×10⁴ g mol⁻¹, up to 6.5×10⁴ g mol⁻¹, up to 6×10⁴ g mol⁻¹, up to 5.5×10⁴ g mol⁻¹, up to 5×10⁴ g mol⁻¹, between 1×10³ g mol⁻¹ and 7.5×10⁶ g mol⁻¹, between 1×10³ g mol⁻¹ and 5×10⁶ g mol⁻¹, between 1×10³ g mol⁻¹ and 1×10⁶ g mol⁻¹, between 1×10³ g mol⁻¹ and 5×10⁵ g mol⁻¹, between 2×10³ g mol⁻¹ and 1×10⁵ g mol⁻¹, between 2×10³ g mol⁻¹ and 1×10⁵ g mol⁻¹, between 3×10³ g mol⁻¹ and 1×10⁵ g mol⁻¹, between 4×10³ g mol- and 1×10⁵ g mol⁻¹, between 4×10³ g mol- and 9.5×10⁴ g mol⁻¹, between 4×10³ g mol⁻¹ and 5.5×10⁴ g mol⁻¹, or between 4×10³ g mol⁻¹ and 5×10⁴ g mol⁻¹.

Optionally, the hydrocarbon polymer in the waste material has a number average molecular weight (Mn) of at least 1×10² g mol⁻¹, at least 5×10² g mol⁻¹, at least 1×10³ g mol⁻¹, at least 1.5×10³ g mol⁻¹, at least 2×10³ g mol⁻¹, at least 2.5×10³ g mol⁻¹, at least 3×10³ g mol⁻¹, at least 3.5×10³ g mol⁻¹, at least 4×10³ g mol⁻¹, up to 2×10⁴ g mol⁻¹, up to 1.5×10⁴ g mol⁻¹, up to 1×10⁴ g mol⁻¹, between 1×10³ g mol⁻¹ and 2×10⁴ g mol⁻¹, between 1.5×10³ g mol⁻¹ and 2×10⁴ g mol⁻¹, between 1.8×10³ g mol⁻¹ and 2×10⁴ g mol⁻¹, between 2×10³ g mol⁻¹ and 2×10⁴ g mol⁻¹, between 3×10³ g mol⁻¹ and 2×10⁴ g mol⁻¹, between 4×10³ g mol⁻¹ and 2×10⁴ g mol⁻¹, between 1.5×10³ g mol- and 1.5×10⁴ g mol⁻¹, or between 1.8×10³ g mol⁻¹ and 1.5×10⁴ g mol−.

For example, the waste material contains a polyethylene having a Mw of at least 3×10³ g mol⁻¹ and optionally a Mn of at least 1.5×10³ g mol⁻¹, such as a Mw in a range between 3×10³ g mol⁻¹ and 1×10⁵ g mol⁻¹ and optionally a Mn in a range between 1.5×10³ g mol⁻¹ and 1.5×10⁴ g mol⁻¹, such as a Mw of about 3.5×10³ g mol⁻¹ and optionally a Mn of about 1.8×10³ g mol⁻¹.

Optionally, the waste material contains a hydrocarbon polymer of different average molecular weights (also referred herein as “molecular weight”). Generally, a low molecular weight hydrocarbon polymer has a Mw in a range from 1000 g mol⁻¹ to 10,000 g mol⁻¹ and a Mn in a range from 100 g mol⁻¹ to 1,000 g mol⁻¹; a medium molecular weight hydrocarbon polymer has a Mw in a range from 10,000 g mol⁻¹ to 200,000 g mol⁻¹ and a Mn in a range from 1,000 g mol⁻¹ to 20,000 g mol⁻¹; a high molecular weight hydrocarbon polymer has a Mw in a range from 200,000 g mol⁻¹ to 500,000 g mol⁻¹ and a Mn in a range from 20,000 g mol⁻¹ to 50,000 g mol⁻¹; and an ultrahigh molecular weight hydrocarbon polymer has a Mw in a range from 500,000 g mol⁻¹ to 7,500,000 g mol⁻¹ and a Mn in a range from 50,000 g mol⁻¹ to 750,000 g mol-. For example, the waste material contains polyethylene, which is a mixture of high molecular weight polyethylene and medium molecular weight polyethylene, a mixture of high molecular weight polyethylene and low molecular weight polyethylene, a mixture of medium molecular weight polyethylene and low molecular weight polyethylene, or a mixture of high, medium, and low molecular weight polyethylene.

Recitation of ranges of values herein are merely intended to serve as a shorthand method of referring individually to each separate value falling within the range, unless otherwise indicated herein, and each separate value is incorporated into the specification as if it were individually recited herein.

b. Number of Carbon Atoms in Hydrocarbon Polymer

Typically, the hydrocarbon polymer(s) in the waste material contains at least 70 carbon atoms, at least 100 carbon atoms, at least 120 carbon atoms, at least 150 carbon atoms, at least 200 carbon atoms, at least 250 carbon atoms, at least 300 carbon atoms, at least 350 carbon atoms, at least 400 carbon atoms, at least 450 carbon atoms, at least 500 carbon atoms, at least 550 carbon atoms, at least 600 carbon atoms, at least 650 carbon atoms, at least 700 carbon atoms, at least 750 carbon atoms, at least 800 carbon atoms, at least 850 carbon atoms, at least 900 carbon atoms, at least 950 carbon atoms, at least 1000 carbon atoms, up to 500,000 carbon atoms, up to 100,000 carbon atoms, up to 50,000 carbon atoms, up to 10,000 carbon atoms, up to 7000 carbon atoms, up to 6500 carbon atoms, up to 6000 carbon atoms, up to 5500 carbon atoms, up to 5000 carbon atoms, up to 4500 carbon atoms, up to 4000 carbon atoms, up to 3500 carbon atoms, between 70 and 500,000 carbon atoms, between 100 and 100,000 carbon atoms, between 120 and 50,000 carbon atoms, between 120 and 10,000 carbon atoms, between 120 and 7000 carbon atoms, between 200 and 7000 carbon atoms, between 500 and 7000 carbon atoms, between 500 and 6500 carbon atoms, between 500 and 6000 carbon atoms, between 500 and 5500 carbon atoms, between 500 and 5000 carbon atoms, between 500 and 4500 carbon atoms, between 500 and 4000 carbon atoms, between 500 and 3500 carbon atoms, or between 3500 and 7000 carbon atoms.

c. Density

Optionally, the waste material contains a hydrocarbon polymer of ultra-low density, very low density, linear low or low density, linear medium or medium density, or high density or a combination thereof. For example, the waste material contains a hydrocarbon polymer with a mixture of low and high densities. Generally, a hydrocarbon polymer of ultra-low density is in the range from 0.867 g cm⁻³ to 0.889 g cm⁻³. A hydrocarbon polymer of very low density is typically in the range from 0.890 g cm⁻³ to 0.914 g cm⁻³. A hydrocarbon polymer of linear low or low density is typically in the range from 0.919 g cm⁻³ to 0.925 g cm⁻³. A hydrocarbon polymer of linear medium or medium density is typically in the range from 0.926 g cm⁻³ to 0.940 g cm⁻³. A hydrocarbon polymer of high density is typically in the range from 0.941 g cm⁻³ to 0.970 g cm⁻³. For example, a polyethylene of ultra-low density is typically in the range from 0.867 g cm 3 to 0.889 g cm⁻³; a polyethylene of very low density is typically in the range from 0.890 g cm⁻³ to 0.914 g cm⁻³; a polyethylene of linear low or low density is typically in the range from 0.919 g cm⁻³ to 0.925 g cm⁻³; a polyethylene of linear medium or medium density is typically in the range from 0.926 g cm⁻³ to 0.940 g cm⁻³; and a polyethylene of high density is in typically the range from 0.941 g cm⁻³ to 0.970 g cm⁻³.

For example, the waste material contains a linear low-density or low-density hydrocarbon polymer, such as a linear low-density or low-density polyethylene having a weight average molecular weight (Mw) in a range from 8.9×10⁴ g mol⁻¹ to 4.7×10⁵ g mol⁻³, such as 94.5×10³ g mol⁻¹ and a density in a range from 0.919 g cm⁻³ to 0.925 g cm⁻³. Exemplary waste material that contains a low-density polyethylene includes freezer bags.

For example, the waste material contains a high-density hydrocarbon polymer, such as a high-density polyethylene having a Mw in a range from 10³ g mol⁻¹ to 10⁷ g mol⁻¹, such as 53.5×10³ g mol⁻¹ and a density in a range from 0.941 g cm⁻³ to 0.970 g cm⁻³. Exemplary waste material that contains a high-density polyethylene includes plastic bottles and plastic packaging, such as a plastic non-biodegradable grocery bag.

Optionally, the waste material contains a hydrocarbon polymer of different densities. For example, the waste material can contain a mixture of high density polyethylene and low density polyethylene.

d. Dispersity

Typically, the waste material contains hydrocarbon polymer having a high dispersity of >1.5, at least 2, at least 2.2, at least 2.5, at least 3, at least 3.5, up to 7.5, up to 7, between 1.8 and 7.5, between 2 and 7.5, or between 3.5 and 7.5, such as at least 1.9, at least 3.6, or at least 7.4.

### 2. Catalysts

The reactor contains a catalyst therein. The catalyst is generally in the form of a solid. Catalysts suitable for upcycling the waste material generally include a transition metal, optionally more than one transition metal. The term “transition metal” refers to a single transition metal or a transition metal that is an element in a compound, such as metal oxide or metal carbide. For example, the catalyst is a transition metal, a mixture of two or more metals containing at least one transition metal, a metal oxide of a transition metal, or a metal carbide of a transition metal, or a combination thereof. The transition metal of the catalyst acts as the catalytic active sites that react with the hydrocarbon polymer of the waste material.

Optionally, the hydrocarbon polymer and transition metal of the catalyst has a ratio (by mass) in a range from 20 to 200, from 30 to 200, from 30 to 150, from 35 to 200, or from 35 to 150, such as about 120.

a. Transition Metals

Optionally, the catalyst is or contains a transition metal, such as platinum, palladium, ruthenium, iridium, rhenium, rhodium, iron, cobalt, nickel, copper, molybdenum, or tungsten. For example, the catalyst is or contains platinum.

Optionally, the catalyst is or contains a mixture of two or more metals containing a transition metal, such as a bimetallic or a trimetallic. For example, the catalyst is a mixture of two or more metals and each metal in the mixture of metals is a transition metal, such as platinum, palladium, ruthenium, iridium, rhodium, iron, cobalt, nickel, copper, molybdenum, or tungsten. For example, the catalyst is a bimetallic of platinum and cobalt.

For example, the catalyst is or contains a mixture of two or more metals containing a first group of transition metal(s) and a second group of metal(s). The first group of transition metals in the mixture of metals contains one or more transition metals, and each of the first group metals is platinum, palladium, ruthenium, iridium, rhodium, iron, cobalt, nickle, copper, molybdenum, or tungsten. The second group of metal(s) in the mixture of metals contains one or more metals that are different from the metals of the first group, and each of the second group metals can be a transition metal or a non-transition metal. For example, each of the second group metals is different from each of the transition metals in the first group and is rhenium, tin, lead, tungsten, molybdenum, chromium, manganese, or zinc. For example, the mixture of metals is a bimetallic containing platinum and rhenium or platinum and tin. For example, the mixture of meals is a trimetallic, and optionally contains platinum, rhenium, and tin.

b. Metal Oxides

Optionally, the catalyst is or contains a metal oxide of a transition metal. The metal oxide can contain a single transition metal or a mixture of two or more metals where at least one of the mixture of metals is a transition metal. For example, the catalyst is a metal oxide of a single transition metal, such as platinum, palladium, ruthenium, iridium, rhodium, iron, cobalt, nickel, copper, molybdenum, or tungsten.

For example, the catalyst is or contains a metal oxide of a mixture of two or more metals and each metal in the mixture of metals is a transition metal, such as platinum, palladium, ruthenium, iridium, rhodium, iron, cobalt, nickel, copper, molybdenum, or tungsten.

For example, the catalyst is or contains a metal oxide of a mixture of two or more metals containing a first group of transition metal(s) and a second group of metal(s) as described above.

Optionally, the catalyst is or contains a metal oxide having a perovskite structure. For example, the catalyst is a metal oxide of a transition metal, such as titanium, niobium, iron, or zirconium, and the catalyst further contains calcium and/or sodium, where the elements of the metal oxide are arranged in a way such that it has the same type of crystal structure as CaTiO₃.

c. Metal Carbides

Optionally, the catalyst is or contains a metal carbide of a transition metal. The metal carbide can contain a single transition metal or a mixture of two or more metals where at least one of the mixture of metals is a transition metal. For example, the catalyst is a metal carbide of a single transition metal, such as platinum, palladium, ruthenium, iridium, rhodium, iron, cobalt, nickel, copper, molybdenum, or tungsten. For example, the metal carbide is molybdenum carbide or tungsten carbide.

For example, the catalyst is or contains a metal carbide of a mixture of two or more metals and each metal in the mixture of metals is a transition metal, such as platinum, palladium, ruthenium, iridium, rhodium, iron, cobalt, nickel, copper, molybdenum, or tungsten.

For example, the catalyst is a metal carbide of a mixture of two or more metals containing a first group of transition metal(s) and a second group of metal(s) described above.

Optionally, more than one catalyst is used in the process. Any catalysts described above may be used. For example, the reactor contains more than one metal oxide described above therein. Each of the metal oxides in the reactor contains a transition metal that is different from the other. For example, the reactor contains more than one metal carbide described above therein. Each of the metal carbides in the reactor contains a transition metal that is different from the other. For example, the reactor contains a metal oxide and a metal carbide therein. The metal oxide in the reactor contains a transition metal that is different from the metal carbide.

d. Form of the Catalyst

The catalyst can be in a variety of suitable forms, such as atoms, nanoclusters, nanoparticles, or a combination thereof.

When the catalyst is in the form of nanoclusters, the nanoclusters can have an average diameter of up to 2 nm, up to 1.5 nm, up to 1 nm, in a range from 0.5 nm to 2 nm, from 0.5 nm to 1.5 nm, from 0.5 nm to 1 nm, or from 1 nm to 2 nm.

When the catalyst is in the form of nanoparticles, the nanoparticles can have an average diameter of up to 50 nm, up to 20 nm, up to 10 nm, up to 5 nm, at least 2 nm, in a range from 2 nm to 50 nm, from 2 nm to 45 nm, from 2 nm to 40 nm, from 2 nm to 35 nm, from 2 nm to 30 mn, from 2 nm to 25 nm, from 2 nm to 20 nm, from 2 nm to 15 nm, from 2 nm to 10 nm, or from 2 nm to 5 nm.

### 3. Other Components

Optionally, the catalyst is dispersed on the surface of a support to form a catalytic system. When a support is present, the catalyst is dispersed on the surface of the support in the form of atoms, nanoclusters, or nanoparticles, or a combination thereof. For example, the catalyst is platinum nanoparticles and the platinum nanoparticles are dispersed on the surface of a silica-alumina support. The support is generally understood to separate the catalyst atoms, nanoclusters, and/or nanoparticles apart and/or adsorb the hydrocarbon polymer(s), thereby improve the efficiency of the depolymerization reaction.

Additionally, some supports, such as acidic support materials or acid-modified support materials, can be included in the catalytic system to increase the efficiency of the catalyst therein. For example, a catalytic reaction using a catalytic system that includes an acidic support produces a liquid product with a similar or higher alkylbenzene yield and a similar or higher alkylbenzene selectivity relative to all aromatic compounds in the liquid product, in a shorter time period, compared to the same catalytic reaction using a catalytic system that does not include the acidic support, under the same reaction conditions. The term “same reaction conditions” means that the catalytic reaction is performed under the same temperature, same pressure, same atmosphere, etc. Optionally, these support materials are catalytic on their own.

Optionally, more than one support is used for forming a catalytic system with the catalyst. When two or more supports are present, the catalyst is dispersed on the surface of at least one of the supports in the form of atoms, nanoclusters, or nanoparticles, or a combination thereof. For example, when two supports are used for forming a catalytic system, the catalyst is platinum nanoparticles and the platinum nanoparticles are dispersed on the surface of a first support; the platinum nanoparticles dispersed first support is mixed with a second support that is different from the first support to form the catalytic system. For example, when two supports are used for forming a catalytic system, the catalyst is platinum nanoparticles and the platinum nanoparticles are dispersed on the surface of a first support and the surface of a second support that is different from the first support; the platinum nanoparticles dispersed first support and second support are mixed to form the catalytic system.

Optionally, when two or more supports are present, a first catalyst can be dispersed on the surface of at least one of the supports in the form of atoms, nanoclusters, or nanoparticles, or a combination thereof, and a second catalyst can be dispersed on the surface of at least one of the supports that is different from the support having the first catalyst dispersed thereon, in the form of atoms, nanoclusters, or nanoparticles, or a combination thereof. For example, when two supports are used for forming a catalytic system, a first catalyst is platinum nanoparticles and the platinum nanoparticles are dispersed on the surface of a first support and a second catalyst that is different from the first catalyst is dispersed on the surface of a second support that is different from the first support.

When two or more supports are present, the weight percentage of each support in the catalytic system is selected based on the specific catalyst and the material of each support. For example, when two supports are used for forming a catalytic system, a first support having platinum nanoparticles dispersed thereon is mixed with a second support without any catalyst dispersed therein, where the second support is different from the first support. The weight percentage of the second support in the catalytic system is in a range from 2% to 90%, from 2% to 50%, from 2% to 35%, from 10% to 90%, from 10% to 80%, from 10% to 70%, from 10% to 60%, from 10% to 50%, from 10% to 30%, from 15% to 40%, from 20% to 50%, from 20% to 40%, or from 20% to 35%, such as about 20% or about 33%, with the balance of the components in the catalytic system being the weight of first support and the weight of the catalyst dispersed thereon.

a. Materials for Support

The material for the support is chemically stable when subject to the operating conditions of the reactor for the depolymerization of the waste material. For example, the material for the support does not react with common chemicals (e.g., hydrogen gas, saturated C1-C₅ alkane, benzene, or toluene, etc.) at the reaction temperature and pressure and for a period of time described herein. For example, the material for the support does not react with common chemicals (e.g., hydrogen gas, saturated C1-C₅ alkane, benzene, or toluene, etc.) at a temperature of 500 °C or less and under a pressure in a range from 1 atm to 8 atm.

Examples of suitable materials for the support include, but are not limited to, oxide compounds, halogenated oxide compounds, metal carbides, metal phosphates, and carbon-based materials, and acid modified oxides, metal carbides, metal phosphates, and carbon-based materials. The material for the support is a different material than the transition metal or mixture of metals in the catalyst.

Optionally, the material for the support is an oxide compound, such as a metal oxide or a non-metal oxide. For example, the oxide compound for the support is silicon dioxide, aluminum oxide (e.g. γ-alumina, amorphous alumina, etc.), halogenated alumina, such as Cl-alumina and F-alumina, silica-aluminum oxide, silica (e.g. silica gel, mesoporous silica, etc.), acid-modified silica, CeO₂, acid-modified CeO₂, TiO₂, acid-modified TiO₂, WO₃, MoO₃, Re₂O₇, a perovskite, clays (dealuminated clays, clays modified with organic or inorganic acids, clays ion exchanged with H⁺, NH4⁺, Fe³⁺, Na⁺, etc.), or a zeolite (e.g. ZSM-5, dealuminated zeolites, zeolites modified by organic and/or inorganic acids, zeolites modified by organic and/or inorganic bases, etc.), or a combination thereof. For example, the oxide compound for the support is silica-aluminum oxide.

Examples of acids that are suitable for modifying the above-mentioned support materials include, but are not limited to, Keggin-type heteropoly acid (H₃PW₁₂O₄₀, H₄SiW₁₂O₄₀, etc.) and their acidic salt thereof (e.g., Cs_{2.5}H_{0.5}PW₁₂O₄₀, etc.). Methods for modifying the support materials are known in the art, such as chemical modifications (e.g., covalently attaching an acid/base on the material) or non-chemical modifications (e.g., physical loading of an acid/base on or entrapping an acid/base in the material). For example, Keggin-type heteropoly acid (H₃PW₁₂O₄₀, H₄SiW₁₂O₄₀, etc.) or an acidic salt thereof (e.g., Cs_{2.5}H_{0.5}PW₁₂O₄₀, etc.) can be physically loaded on a silica-aluminum oxide support, a silica support, a TiO₂ support, or a carbon-based material.

Optionally, the material for the support is a metal carbide. For example, the metal carbide forming the support is any metal carbide described above, such as molybdenum carbide or tungsten carbide.

Optionally, the material for the support is a metal phosphate. The metal phosphate forming the support can contain a transition metal or a mixture of metals described above for metal oxides.

Optionally, the material for the support is a carbon-based material. A carbon-based material generally refers to a material where the number of carbon atoms are at least 50% of the total number of atoms in the material. Examples of suitable carbon-based materials for the support include, but are not limited to, graphite, graphite oxide, activated carbon, carbon nanotubes, carbon nanosheet, graphene, and graphene oxide, and an acid modified version thereof, or a combination thereof. Suitable acids for modifying these carbon-based materials can be any of the inorganic acids described above, such as Keggin-type heteropoly acid (H₃PW₁₂O₄₀, H₄SiW₁₂O₄₀, etc.) and their acidic salt thereof (e.g. Cs₂₅H_{0.5}PW₁₂O₄₀, etc.).

Optionally, the material for the support is a Keggin-type heteropoly acid (H₃PW₁₂O₄₀, H₄SiW₁₂O₄₀, etc.) or an acidic salt of the Keggin-type heteropoly acid (Cs_{2.5}H_{0.5}PW₁₂O₄₀, etc.).

The material for the support may be catalytic or non-catalytic. For example, the support is formed from a catalytic material that can participate in the depolymerization reaction. In these cases, the catalytic material for the support may be a transition metal, a mixture of metals containing at least one transition metal, a metal oxide containing a transition metal, or a metal carbide containing a transition metal as described above mass suitable materials for the catalyst. However, the catalytic material for the support is different from the metal in the catalyst. Optionally, the material for the support is a material that does not participate in the depolymerization reaction, such as silica oxide or a carbon-based material. For example, the material for the support is γ-alumina or silica-alumina.

Optionally, the material for the support is an acidic material. Typically, the acidic material is strong enough to protonate pyridine, as determined by known methods. Whether a given material is able to protonate pyridine, and therefore considered to be an acidic material can be shown using the following test. First, the material is dehydrated at about 400 °C under vacuum; then the dehydrated material is dosed with an excess of pyridine vapor at room temperature, followed by desorption, typically at about 150 °C under vacuum for suitable time period, such as about 20 minutes. If the IR spectra of the resulting material shows a peak at 1545 cm⁻¹, strong Brønsted acid sites are present, and thus demonstrates its acidity for protonating pyridine. Examples of suitable acidic materials that can be used for the support include, but are not limited to, halogenated oxide compounds (e.g. Cl-alumina and F-alumina), WO₃, MoO₃, Re₂O₇, acid modified oxide compounds, such as acid-modified zeolites, silica, CeO₂, TiO₂, clays, etc., and acid modified carbon-based materials (e.g. acid-modified activated carbon, carbon nanotubes, graphene, etc.), where the acid can be any of the acids described above. When the material for the support is a halogenated oxide compound, such as Cl-alumina or F-alumina, the weight percentage of the halogen in the support can be in a range from 0.1% to 5%, from 0.5% to 3%, from 0.5% to 2%, from 0.5% to 1.5%, such as about 0.7% or about 1.3%.

Optionally, when a single support is used for forming a catalytic system, such as any one of the oxide compounds, halogenated oxide compounds, metal carbides, metal phosphates, and carbon-based materials, and their acid modified versions thereof described above, the weight percentage of the support in the catalytic system is in a range from 90% to 99.9%, from 90% to 99.8%, from 90% to 99.5%, from 90% to 99%, from 98% to 99.9%, from 98% to 99.8%, from 98% to 99.5%, from 98% to 99%, from 98.5% to 99.9%, from 98.5% to 99.8%, from 98.5% to 99.5%, or from 99% to 99.9%, with the balance of the components in the catalytic system being the weight of the catalyst dispersed thereon.

Optionally, when two or more supports are used for forming a catalytic system, at least one of the supports is an acidic support as described above, such as a halogenated oxide compound (e.g., Cl-alumina and F-alumina). In these catalytic systems, the catalyst can be dispersed on the surface of the acidic support(s) and/or on the surface of the support material that is different from the acidic support(s).

For example, when two supports are used for forming a catalytic system, a first support is γ-alumina or silica-alumina having the catalyst dispersed thereon and a second support is an acidic support as described above, such as F-alumina, that does not have the catalyst dispersed thereon. The weight percentage of the acidic support in the catalyst system is in a range from 2% to 90%, from 2% to 50%, from 2% to 35%, from 10% to 90%, from 10% to 80%, from 10% to 70%, from 10% to 60%, from 10% to 50%, from 10% to 30%, from 15% to 40%, from 20% to 50%, from 20% to 40%, or from 20% to 35%, such as about 20% or about 33%, with the balance of the components in the catalytic system being the weight of the first support and the weight of the catalyst dispersed thereon.

For example, when two supports are used for forming a catalytic system, a first support is γ-alumina or silica alumina having the catalyst dispersed thereon and a second support is an acidic support as described above, such as F-alumina, that also has the catalyst dispersed thereon. The weight percentage of the acidic support in the catalyst system is in a range from 2% to 90%, from 2% to 50%, from 2% to 35%, from 10% to 90%, from 10% to 80%, from 10% to 70%, from 10% to 60%, from 10% to 50%, from 10% to 30%, from 15% to 40%, from 20% to 50%, from 20% to 40%, or from 20% to 35%, such as about 20% or about 33%, with the balance of the components in the catalytic system being the weight of the first support and the weight of the catalyst dispersed on the first and second supports. The catalyst dispersed on the first support can be the same as or different from the catalyst that is dispersed on the second support.

b. Forms of the Support

Typically, the support has a surface area having a dimension sufficiently large to allow the catalyst to be dispersed hereon. When calculating surface area of the support, any surface that the reactants are able to contact is typically included. Surface area of the support can be measured by techniques known in the art, for example, by nitrogen physisorption. Typically, the support has a surface area of at least 50 m² g⁻¹, at least 100 m² g⁻¹, at least 150 m² g⁻¹, at least 500 m² g⁻¹, in a range from 50 m² g⁻¹ to 1000 m² g⁻¹, from 50 m² g⁻¹ to 900 m² g⁻¹, from 50 m² g⁻¹ to 800 m² g⁻¹, from 50 m² g³ to 700 m² g⁻¹, from 50 m² g⁻¹ to 600 m² g⁻¹, from 50 m² g⁻¹ to 500 m² g⁻¹, from 100 m² g⁻¹ to 1000 m² g⁻¹, from 100 m² g⁻¹ to 800 m² g⁻¹, from 100 m² g⁻¹ to 600 m² g⁻¹, or from 150 m² g⁻¹ to 800 m² g⁻¹. For example, the support has a surface area of at least 150 m² g⁻¹, such as 186 m² g⁻¹ or 573 m² g⁻¹.

The support can be in a form of a mesoporous sheet or microparticles, or a combination thereof. For example, the support can be in the form of aluminum oxide microparticles. In some embodiments, the support can be in the form of a carbon sheet (e.g., graphene sheet), carbon powders, carbon particles, or carbon nanoparticles (e.g., carbon nanotubes).

The support is generally understood to stabilize the metal atoms, nanoclusters, and/or nanoparticles that are one active phase of the catalyst by slowing their aggregation, and/or to adsorb the hydrocarbon polymer(s). By separating the active metal atoms of the catalyst using the support, the accessible active surface area of the catalyst is increased prior to reaction and mostly preserved during reaction, thereby improving the efficiency of the depolymerization reaction. For example, when the catalyst is a metal or a mixture of metals dispersed on the support in the form of atoms, each atom is displaced a distance or different distances from the other atoms of the metal or mixture of metals. For example, when the catalyst is dispersed on the support in the form of nanoclusters, each nanocluster is displaced a distance or different distances from the other nanoclusters of the catalyst. For example, when the catalyst is dispersed on the support in the form of nanoparticles, each nanoparticle is displaced a distance or different distances from the other nanoparticles of the catalyst. The dispersion of the catalyst on the support can be measured by methods known in the art, such as by CO chemisorption using a Micrometrics Autochem ii 2920.

c. Weight Loading

Generally, the total weight loading of the one or more metals of the catalyst is present on the support in an amount less than 10 wt % of the total weight of the catalyst and the support. Methods for measuring the weight loading of the metal(s) of the catalyst present on the support are known, such as by using Inductively-Coupled Plasma—Optical Emission Spectrometry (ICP-OES). For example, when the catalyst includes one or more transition metals, the total weight loading of the metals present in the catalyst on the support is calculated as the sum of the weights of the transition metals in the catalyst divided by the sum of the weight of the support plus the weights of the transition metals and non-transition metals multiplied by 100. When the catalyst includes only one transition metal, the total weight loading of the metals present in the catalyst on the support is simply calculated as the weight of the transition metal in the catalyst divided by the sum of the weight of the support plus the weight of the transition metal multiplied by 100. Similarly, when the catalyst includes both one or more transition metals and one or more non-transition metals, the total weight loading of the metals present in the catalyst on the support is calculated as the sum of the weights of transition metals and non-transition metals in the catalyst divided by the sum of the weight of the support plus the weights of the transition metals and non-transition metals multiplied by 100.

For example, the total weight loading of the one or more metals of the catalyst is present on the support in an amount of less than 10 wt %, less than 8 wt %, less than 5 wt %, less than 4.5 wt %, less than 4 wt %, less than 3.5 wt %, less than 3 wt %, less than 2.5 wt %, less than 2 wt %, less than 1.5 wt %, less than 1 wt %, in a range of from 0.1 wt % to 10 wt %, from 0.1 wt % to 8 wt %, from 0.1 wt % to 5 wt %, from 0.2 wt % to 5 wt %, from 0.5 wt % to 10 wt %, from 0.5 wt % to 8 wt %, from 0.5 wt % to 5 wt %, from 1 wt % to 10 wt %, from 1 wt % to 8 wt %, from 1 wt % to 5 wt %, from 0.1 wt % to 4.5 wt %, from 0.1 wt % to 4 wt %, from 0.1 wt % to 3.5 wt %, from 0.1 wt % to 3 wt %, from 0.1 wt % to 2.5 wt %, or from 0.1 wt % to 2 wt %, from 0.1 wt % to 1.5 wt %, from 0.1 wt % to 1 wt %, or from 0.1 wt % to 0.5 wt % of the total weight of the catalyst and the support, such as about 1.7 wt % of the total weight of the catalyst and the support.

Methods for preparing the disclosed catalysts are known. For example, the disclosed catalysts may be prepared using the methods described in Garcia and Goto, Mater. Trans., 44(9):1717-1728 (2003).

### B. Operate the Reactor to Convert the Hydrocarbon Polymer to a Product.

The reactor is operated at a sufficient temperature, under a sufficient H₂ pressure, and for a sufficient period of time, to convert the hydrocarbon polymer to a product. The hydrocarbon polymer is converted to the product via depolymerization reactions. Optionally, the depolymerization reaction in step (ii) is performed under stirring using methods known in the art, such as magnetic stirring.

The product contains a liquid and the liquid contains an alkylbenzene compound, optionally more than one alkylbenzene compound. Optionally, the liquid product also contains other aromatic compounds, such as one or more alkylpolyaromatic compounds and/or one or more polyaromatic compounds. Optionally, in addition to the alkylbenzene compound(s) and optionally the other aromatic compound(s), the liquid product also contains one or more alkanes and/or one or more non-aromatic unsaturated compounds (such as olefins). The alkylbenzene compound(s), other aromatic compound(s) (when present), alkane(s) (when present), and non-aromatic unsaturated compounds (when present) in the liquid product each contains from 7 to 40 carbon atoms.

When one or more other aromatic compounds are present in the liquid product, the mass selectivity of the alkylbenzene compound(s) relative to all aromatic compounds in the liquid product is at least 25%, at least 30%, at least 50%, in a range from 25% to 99.99%, from 25% to 99.9%, from 25% to 99%, from 25% to 95%, from 25% to 90%, from 25% to 90%, from 25% to 80%, from 25% to 70%, or from 25% to 60%. The mass selectivity of alkylbenzene compound(s) relative to all aromatic compounds in the liquid product is calculated as the sum of the weights of the alkylbenzene compound(s) in the liquid product relative to the sum of the weights of all aromatic compounds in the liquid product, which includes alkylbenzene compound(s) and other aromatic compound(s) that is/are not an alkylbenzene compound, as a percentage. When the liquid product contains alkylbenzene compound(s) as the only aromatic compound(s), the selectivity of the alkylbenzene compound(s) is simply 100%.

Typically, the reactor is operated at a low temperature, such as less than or equal to 500 °C, optionally less than or equal to 450 °C, less than or equal to 400 °C, less than or equal to 350 °C, or less than or equal to 300 °C. Optionally, the process includes, prior to step (ii), preheating the reactor to the depolymerization temperature in step (ii).

1. Hydrogen gas pressure

In step (ii), the reactor can be operated under a constant H₂ pressure or varied H₂ pressures for the duration of the depolymerization reaction to form the liquid product containing alkylbenzene compound(s).

The constant H₂ pressure or the first H₂ pressure (when varied H₂ pressures are used) in the reactor can be achieved by feeding hydrogen gas into the reactor during step (i), feeding the waste material into the reactor, or after step (i) and prior to the step of operating the reactor to depolymerize the waste material contained in the reactor. The H₂ pressure under which the reactor operates may be measured immediately prior to the depolymerization reaction in step (ii) or during step (ii).

For example, as shown in FIG. 12, hydrogen gas is fed into the reactor 10 via a hydrogen gas stream 300 simultaneously or substantially simultaneously with feeding the waste material, or subsequent to feeding the waste material and prior to the depolymerization reaction.

Following the hydrogen gas feeding step, the desired H₂ pressure for performing depolymerization reaction of the plastic waste material is achieved in the reactor.

Optionally, the hydrogen gas is purified prior to being fed into the reactor. Methods for purifying hydrogen gas are known, for example, by passing through 13× molecular sieves to remove water and BTS catalyst to remove oxygen.

Typically, the H₂ pressure(s) under which the reactor is operated is effective to achieve the above-described alkylbenzene mass selectivity, i.e., an alkylbenzene mass selectivity of at least 25% relative to all aromatic compounds in the liquid product.

For example, the reactor operates under a constant H₂ pressure in a range from 1 atm to 8 atm, from 1 atm to 7 atm, from 1 atm to 6 atm, from 1 atm to 5 atm, or from 1 atm to 4 atm for depolymerizing a plastic waste material contained therein, and the liquid product obtained from the depolymerization reaction contains alkylbenzene compound(s) with a mass selectivity of at least 25% relative to all aromatic compounds in the liquid product.

For example, the reactor operates under two different H₂ pressures, such as a first P(H₂) ranging from 5 atm to 20 atm, from 5 atm to 18 atm, from 5 atm to 15 atm, from 5 atm to 12 atm, from 5 atm to 10 atm, and a second P(H₂) ranging from 0 to <5 atm, from 0 to 4 atm, from 0 to 3 atm, or from 0 to 2 atm for depolymerizing a plastic waste material contained therein, and the liquid product obtained from the depolymerization reaction contains alkylbenzene compound(s) with a mass selectivity of at least 25% relative to all aromatic compounds in the liquid product.

For example, the reactor operates under three different H₂ pressures, such as a first P(H₂) ranging from 5 atm to 20 atm, from 5 atm to 18 atm, from 5 atm to 15 atm, from 5 atm to 12 atm, from 5 atm to 10 atm; a second P(H₂) ranging from 0 to <5 atm, from 0 to 4 atm, from 0 to 3 atm, or from 0 to 2 atm; and a third P(H₂) ranging from 5 atm to 15 atm, from 5 atm to 12 atm, or from 5 atm to 10 atm, for depolymerizing a plastic waste material contained therein, and the liquid product obtained from the depolymerization reaction contains alkylbenzene compound(s) with a mass selectivity of at least 25% relative to all aromatic compounds in the liquid product.

a. Constant Hydrogen Gas Pressure

In some forms, in step (ii), the reactor is operated under a constant H₂ pressure to form the product. When a constant H₂ pressure is applied, hydrogen gas may be fed into the reactor during step (ii), one or more times, to maintain the constant P(H₂). In these instances, P(H₂) can be measured continuously or at regular intervals during the depolymerization reaction such that the desired P(H₂) is maintained during the reaction. For example, the P(H₂) in the reactor is maintained during the reaction in step (ii) at a value ranging from 1 atm to 8 atm, from 1 atm to 7 atm, from 1 atm to 6 atm, from 1 atm to 5 atm, or from 1 atm to 4 atm, such as 8 atm.

b. Varied Hydrogen Gas Pressures

In some forms, in step (ii), the reactor is operated under two or more different H₂ pressures to form the product.

i. Two Different Hydrogen Gas Pressures

For example, the reactor is operated under two different H₂ pressures to produce the product, and step (ii) of the process includes step (iia) operating the reactor under a first P(H₂) to form fragments of the hydrocarbon polymer, and after step (iia), step (iib) operating the reactor under a second P(H₂) to form the product. Typically, the second P(H₂) is lower than the first P(H₂). For example, the first P(H₂) ranges from 5 atm to 20 atm, from 5 atm to 18 atm, from 5 atm to 15 atm, from 5 atm to 12 atm, from 5 atm to 10 atm, such as about 8 atm, and/or the second P(H₂) ranges from 0 to <5 atm, from 0 to 4 atm, from 0 to 3 atm, or from 0 to 2 atm.

When the P(H₂) in the reactor is varied from a first P(H₂) to a second P(H₂) that is lower than the first P(H₂), the process can further include in step (iib) removing hydrogen gas from the reactor to provide the second P(H₂). In these forms, when the reactor is operated under the first P(H₂), the plastic waste material contained therein is depolymerized to form fragments of the hydrocarbon polymer having lower weight average molecular weight (Mw).

Typically, the fragments of the hydrocarbon polymers are measured continuously or at regular intervals during step (iia) under the first P(H₂) to determine the weight average molecular weight (Mw) of the fragments. Methods for measuring the fragments of the hydrocarbon polymers to determine Mw during step (iia) are known. For example, a sample of the hydrocarbon polymer fragments is obtained from the reactor continuously or periodically and subject to measurement and analysis using a suitable instrument, such as NMR, Mass-spectrometry, etc.

Generally, when the fragments of the hydrocarbon polymer obtained under the first P(H₂) reach an average chain length of <100 carbon atoms, the P(H₂) in the reactor is lowered to the second P(H₂), such as by removing the hydrogen gas from the reactor.

When the reactor is operated under the second P(H₂), the liquid product that contains alkylbenzene compound(s) is formed. In some forms, the second P(H₂) can be 0. In these forms, the reactor in step (iib) is operated under a pressure by an inert gas, such as an argon pressure. The inert gas pressure can range from 0 to <5 atm, from 0 to 4 atm, from 0 to 3 atm, or from 0 to 2 atm. When an inert gas is used to provide a pressure in the reactor, the process includes, in step (iib), removing hydrogen gas from the reactor and feeding the inert gas into the reactor to provide the inert gas pressure.

ii. Three Different Hydrogen Gas Pressures

In some forms, the reactor is operated under three different H₂ pressures to produce the product. For example, step (ii) of the process includes step (iia) operating the reactor under a first P(H₂) to form fragments of the hydrocarbon polymer, after step (iia), step (iib) operating the reactor under a second P(H₂) to form aromatic compounds, and after step (iib), step (iic) operating the reactor at a third P(H₂) to form the product. Typically, the second P(H₂) is lower than the first P(H₂), and the third P(H₂) is higher than the second P(H₂). For example, the first P(H₂) and second P(H₂) each are in a range as described above; and the third P(H₂) ranges from 5 atm to 15 atm, from 5 atm to 12 atm, or from 5 atm to 10 atm.

The reaction under the first P(H₂), methods for determining the reaction stage for pressure change, and methods for adjusting H₂ pressure from the first P(H₂) to the second P(H₂) and optionally providing an inert gas pressure in step (iib) are as described above.

When the reactor is operated under the second P(H₂), aromatic compounds that contain alkylbenzenes, polyaromatics, etc. are formed. Typically, in step (iib), the aromatic compounds is measured continuously or at regular intervals to determine the total yield of the aromatic compounds. Methods for measuring the aromatic compounds to determine their total yield during step (iib) are known. For example, a sample of the aromatic compounds is obtained from the reactor continuously or periodically and subject to measurement and analysis using a suitable instrument, such as ¹H NMR spectroscopy combined with gas chromatography. More specific methods for measuring and analyzing a sample of the aromatic compounds to determine their total yield are described in the Examples below.

Generally, when the total yield of the aromatic compounds obtained under the second P(H₂) reach a plateau, the P(H₂) in the reactor is increased to provide the third P(H₂) in step (iic), such as by feeding hydrogen gas into the reactor. When an inert gas is used in step (iib) to provide an inert gas pressure, the inert gas can be removed from the reactor using any suitable method and hydrogen gas can be fed into the reactor simultaneously with or subsequently to the inert gas removal process. In some forms, the flow of hydrogen gas into the reactor can replace any inert gas remained in the reactor and thereby remove the inert gas from the reactor. In some forms, even an inert gas is used in step (iib) to provide an inert gas pressure, in step (iic) after step (iib), the reactor can be pressurized with H₂ without removing the inert gas from the reactor.

When the reactor is operated under the third P(H₂), the liquid product that contains alkylbenzene compound(s) is formed. Generally, the third P(H₂) used for operating the reactor in step (iic) can convert the polyaromatics in the aromatic compounds formed in step (iib) to alkylbenzenes and thereby increase the yield of alkylbenzenes and remove undesired polyaromatics in the product.

### 2. Exemplary Process

An exemplary upcycling process, which includes the steps of (i) feeding a waste material containing hydrocarbon polymer(s) into a reactor; and (ii) operating the reactor at a sufficient temperature, under a sufficient H₂ pressure, and for a sufficient period of time to convert the hydrocarbon polymer to a product, is schematically illustrated in FIG. 12. As shown in FIG. 12, the process for upcycling a waste material typically includes feeding the plastic waste material containing one or more hydrocarbon polymer(s) into a reactor 10 via waste material stream 100. The reactor 10 contains a catalyst therein. The catalyst is fed into the reactor via the catalyst stream 200. Hydrogen gas is fed into the reactor 10 via the hydrogen gas stream 300. The hydrogen gas can be fed into the reactor simultaneously or subsequent to feeding the waste material into the reactor 10. Optionally a reagent/solvent is fed into the reactor 10 via reagent stream 210. The catalyst and optionally the reagent/solvent can be fed into the reactor 10 prior to, simultaneously, or subsequent to feeding the waste material into the reactor 10. Following step (i), the waste material is in contact with the catalyst in the reactor 10, and the reactor is under a sufficient H₂ pressure for the depolymerization reaction to form a product. The H₂ pressure in reactor 10 can be maintained at a constant value or varied. When the H₂ pressure in reactor 10 is varied, hydrogen gas in the reactor can be removed from the reactor such as via the gas stream 300 to lower the H₂ pressure in reactor during the reaction. Additionally and alternatively, hydrogen gas can be added into the reactor optionally via the gas stream 300 to increase the H₂ pressure in reactor during the reaction. The hydrogen gas addition and/or removal process can be repeated one or more times. Optionally, an inert gas can be fed into the reactor 10 via an inert gas stream (not shown in FIG. 12) to adjust the H₂ pressure in the reactor. For example, during the reaction to form the product, hydrogen can be removed from the reactor 10 via the gas stream 300 or a separate gas outlet, and an inert gas can be fed into the reactor to achieve a suitable inert gas pressure. During the reaction, the reactor 10 is heated via heating path 220 to a sufficient temperature and maintained at this temperature for a sufficient period of time to convert the hydrocarbon polymer to the product stream 110, which contains a liquid and optionally a wax. When wax is present in the product stream 110, the liquid and wax can be separated by passing the mixture through a separation unit 20.

The liquid of the product stream 110 contains an alkylbenzene compound or more than one alkylbenzene compound. Optionally, the liquid of the product stream 110 also contains other aromatic compounds (such as one or more alkylpolyaromatic compounds and/or one or more polyaromatic compounds), one or more alkanes, and/or one or more non-aromatic unsaturated compounds (such as one or more olefins), in addition to the alkylbenzene compound(s). The mass selectivity of the alkylbenzene compound(s) in the liquid of the product stream 110 is at least 25% relative to all aromatic compounds in the liquid. In addition to the product stream 110 containing the liquid and optionally the wax, gas products (light C1-C6 hydrocarbons and other gas such as hydrogen gas) also exit the reactor. Additionally, some unreacted solid waste may remain. The solid waste can be removed from the reactor 10 via the organic solid stream 130. The gas product containing hydrogen gas and/or a short-chain hydrocarbon(s), such as a saturated C₁-C₅ alkane, benzene, or toluene, or a combination thereof exits the reactor via gas stream 120.

Optionally, the product stream 110 is fed to a separation unit 20 where the liquid and optionally the wax is separated into short-chain (i.e. C₆-C₁₀) aromatic compound(s) and optionally short-chain (i.e. C₆-C₁₀) alkane stream 111, alkane stream 112 containing a mixture of alkanes, such as alkanes with carbon numbers ranging from 11 to 80 or from 16 to 22, and/or alkylaromatic compound stream 113 containing alkylaromatic compound(s) with average carbon numbers described below, such as alkylaromatic compound(s) with an average carbon number of about 20. The alkylaromatic compound stream 113 contains one or more alkylbenzene compounds and optionally one or more alkylpolyaromatic compounds. Although not illustrated, when alkylpolyaromatic compounds are present in the product stream 110, the alkylpolyaromatic compounds can be separated from the alkylbenzene compounds by passing through the separation unit such that the alkylaromatic compound stream 113 only contains the alkylbenzene compounds, or the alkylaromatic compound stream 113 containing alkylbenzene compounds and alkylpolyaromatic compounds are further separated to produce an alkylbenzene stream.

### 3. Product

Following steps (i) and (ii), product containing a liquid (also referred to herein as “liquid product”) and optionally a wax, is formed. The product containing a liquid and optionally a wax is identified as stream 110 in FIG. 12. Optionally, the wax (when present) in the product contains long chain alkanes with a carbon number from 41 to 100 and optionally can become a flowable liquid when heated to a temperature of at least 45 °C.

The liquid product contains an alkylbenzene compound, optionally more than one alkylbenzene compound. For example, the liquid product contains a mixture of two alkylbenzene compounds, a mixture of three alkylbenzene compounds, a mixture of four alkylbenzene compounds, a mixture of five alkylbenzene compounds, and or a mixture of more than five different alkylbenzene compounds. Optionally, the liquid product contains other aromatic compounds that are not alkylbenzene compounds (such as alkylpolyaromatic compounds and polyaromatic compounds), alkanes (e.g., alkanes with carbon numbers ranging from 7 to 40), and/or non-aromatic unsaturated compounds, such as olefins with carbon numbers ranging from 7 to 40.

Optionally, the liquid product includes xylene, ethylbenzene, propylbenzene, tetramethylbenzene, or butylbenzene, or a combination thereof, and the mass selectivity of these alkylbenzene compounds is at least 15% relative to all aromatic compounds in the liquid product . . . .

a. Alkylbenzene Compounds

The liquid product contains an alkylbenzene compound, and optionally contains more than one alkylbenzene compound. For example, the liquid product contains a mixture of two alkylbenzene compounds, a mixture of three alkylbenzene compounds, a mixture of four alkylbenzene compounds, a mixture of five alkylbenzene compounds, and or a mixture of more than five different alkylbenzene compounds. After exiting the reactor, the alkylbenzene compounds can be separated from the other compounds in the product, such as alkylpolyaromatic compounds, polyaromatic compounds, C7-C40 alkanes, and/or C7-C40 olefins in the liquid and optionally C41-C100 alkanes in the wax, in the product stream by passing through a separation unit. For example, in the exemplary process shown in FIG. 12, the separation unit 20 separates the compounds in the product stream 110 to produce an alkylaromatic compound(s) stream 113 that predominantly contains alkylbenzene compound(s) (such as >99% by weight). Alternatively, for example, although not illustrated in FIG. 12, the alkylaromatic stream 113 contains both alkylbenzene compound(s) and alkylpolyaromatic compound(s), which are further separated to produce an alkylbenzene stream.

i. Number of Carbons and Average

### Molecular Weight

Typically, the alkylbenzene compound or each alkylbenzene compound in the liquid product contains at least 7 carbon atoms and up to 40 carbon atoms, such as in a range from 10 to 30 carbon atoms.

Typically, the alkylbenzene compound or each alkylbenzene compound in the liquid product has a Mw less than 800 g mol⁻¹, less than 750 g mol⁻¹, less than 700 g mol⁻¹, less than 650 g mol−, less than 600 g mol⁻¹, less than 550 g mol⁻¹, less than 500 g mol⁻¹, at least 300 g mol⁻¹, at least 200 g mol⁻¹, at least 150 g mol⁻¹, in a range of from 150 g mol⁻¹ to 800 g mol⁻¹, from 200 g mol⁻¹ to 800 g mol⁻¹, from 200 g mol⁻¹ to 600 g mol⁻¹, from 200 g mol⁻¹ to 500 g mol⁻¹, from 200 g mol⁻¹ to 400 g mol⁻¹, or from 200 g mol⁻¹ to 400 g mol⁻¹. For example, the alkylbenzene compound or each alkylbenzene compound in the liquid product has a Mw ranging from 200 g mol−³ to 400 g mol⁻¹.

Optionally, the alkylbenzene compound or each alkylbenzene compound in the liquid product has a Mn less than 700 g mol−, less than 650 g mol⁻¹, less than 600 g mol−, less than 550 g mol⁻¹, less than 500 g mol⁻¹, at least 300 g mol⁻¹, at least 200 g mol⁻¹, at least 150 g mol⁻¹, in a range of from 150 g mol⁻¹ to 700 g mol⁻¹, from 200 g mol⁻¹ to 700 g mol⁻¹, from 200 g mol⁻¹ to 600 g mol⁻¹, from 200 g mol⁻¹ to 500 g mol⁻¹, from 300 g mol⁻¹ to 700 g mol⁻¹, from 300 g mol⁻¹ to 600 g mol⁻¹, from 300 g mol⁻¹ to 500 g mol⁻¹, or from 200 g mol⁻¹ to 400 g mol⁻¹. For example, the alkylbenzene compound or each alkylbenzene compound in the liquid product has a Mn ranging from 200 g mol⁻¹ to 400 g mol⁻¹.

Optionally, the alkylbenzene compound or each alkylbenzene compound in the liquid product has a Mw or Mn that is at least 5-times less than the Mw or Mn of the hydrocarbon polymer (reactant) in the waste material before depolymerization, at least 10-times less than the Mw or Mn of the hydrocarbon polymer, at least 15-times less than the Mw or Mn of the hydrocarbon polymer, at least 20-times less than the Mw or Mn of the hydrocarbon polymer, at least 25-times less than the Mw or Mn of the hydrocarbon polymer, at least 30-times less than the Mw or Mn of the hydrocarbon polymer, at least 35-times less than the Mw or Mn of the hydrocarbon polymer, at least 40-times less than the Mw or Mn of the hydrocarbon polymer, at least 45-times less than the Mw or Mn of the hydrocarbon polymer, at least 50-times less than the Mw or Mn of the hydrocarbon polymer, at least 55-times less than the Mw or Mn of the hydrocarbon polymer, at least 60-times less than the Mw or Mn of the hydrocarbon polymer, at least 65-times less than the Mw or Mn of the hydrocarbon polymer, at least 70-times less than the Mw or Mn of the hydrocarbon polymer, at least 75-times less than the Mw or Mn of the hydrocarbon polymer, at least 80-times less than the Mw or Mn of the hydrocarbon polymer, at least 85-times less than the Mw or Mn of the hydrocarbon polymer, at least 90-times less than the Mw or Mn of the hydrocarbon polymer, at least 95-times less than the Mw or Mn of the hydrocarbon polymer, at least 100-times less than the Mw or Mn of the hydrocarbon polymer, at least 120-times less than the Mw or Mn of the hydrocarbon polymer, at least 150-times less than the Mw or Mn of the hydrocarbon polymer, or at least 200-times less than the Mw or Mn of the hydrocarbon polymer.

For example, the alkylbenzene compound or each alkylbenzene compound in the liquid product has a Mw at least 5-times less than the Mw of the hydrocarbon polymer (reactant) of the waste material before depolymerization, at least 10-times less than the Mw of the hydrocarbon polymer (reactant), at least 20-times less than the Mw of the hydrocarbon polymer (reactant), at least 50-times less than the Mw of the hydrocarbon polymer (reactant), at least 100-times less than the Mw of the hydrocarbon polymer (reactant), at least 150-times less than the Mw of the hydrocarbon polymer (reactant), or at least 200-times less than the Mw of the hydrocarbon polymer (reactant).

For example, the alkylbenzene compound or each alkylbenzene compound in the liquid product has a Mn at least 5-times less than the Mn of the hydrocarbon polymer (reactant) of the waste material before depolymerization, at least 10-times less than the Mn of the hydrocarbon polymer (reactant), at least 20-times less than the Mn of the hydrocarbon polymer (reactant), at least 50-times less than the Mn of the hydrocarbon polymer (reactant), at least 100-times less than the Mn of the hydrocarbon polymer (reactant), at least 150-times less than the Mn of the hydrocarbon polymer (reactant), or at least 200-times less than the Mn of the hydrocarbon polymer (reactant).

ii. Dispersity

Generally, the dispersity (polydispersity index, PDI) of the distribution of the molecular weight of the alkylbenzene compound in the liquid product is smaller than the dispersity of the distribution of the molecular weight of the hydrocarbon polymer (reactant) prior to depolymerization. Typically, the alkylbenzene compound in the liquid product has a dispersity of less than 4.0, less than 3.5, less than 3.0, less than 2.5, less than 2.0, less than 1.5, less than 1.3, less than 1.2, between 1.1 and 1.5, between 1.1 and 1.4, between 1.1 and 1.3, between 1.1 and 1.2, or between 1.0 and 1.1.

iii. Structure of the Alkylbenzene Compounds

The alkylbenzene compound or each alkylbenzene compound in the liquid product can contain two alkyl groups, three alkyl groups, four alkyl groups, five alkyl groups, or six alkyl groups, attached to the benzene ring. For example, the alkylbenzene compound or each alkylbenzene compound in the liquid product can contain two alkyl groups, three alkyl groups, or four alkyl groups, attached to the benzene ring. Generally, the alkyl group of the alkylbenzene compound in the liquid product can be linear, branched, or cyclic.

For example, the alkyl group of the alkylbenzene compound or each alkylbenzene compound in the liquid product can be a linear C₁-C₂₀ alkyl, a branched C₄-C₂₀ alkyl, or a cyclic C₃-C₂₀ alkyl, optionally, a linear C₁-C₁₅ alkyl, a branched C₄-C₁₅ alkyl, or a cyclic C₃-C₁₅ alkyl, optionally a linear C₁-C₁₂ alkyl, a branched C₄-C₁₂ alkyl, or a cyclic C₃-C₁₂ alkyl, optionally a linear C₁-C₁₀ alkyl, a branched C₄-C₁₀ alkyl, or a cyclic C₃-C₁₀ alkyl, optionally a linear C₁-C₅ alkyl, a branched C₄-C₅ alkyl, or a cyclic C₃-C₅ alkyl.

Optionally, the alkyl group of the alkylbenzene compound or each alkylbenzene compound in the liquid product is a linear C₁-C₂₀, C1-C₁₅, C₁-C₁₂, C₁-C₁₀, C₁-C₈, C₁-C₅, C₂-C₂₀, C₂-C₁₅, C₂-C₁₂, C2-C10, C2-C₅, C₂-C₅, C₃-C₂₀, C₃-C₁₅, C₃-C₁₂, C3-C10, C3-C₅, C₃-C₅, C₄-C₂₀, C₄-C₁₅, C₄-C₁₂, C₄-C₁₀, C₄-C₅, C₄-C₅, C₅-C₂₀, C₅-C₁₅, C₅-C₁₂, C₅-C₁₀, or C₅-C₅ alkyl group.

Optionally, the alkylbenzene compound or each alkylbenzene compound in the liquid product contains one, two, three, four, or five alkyl groups attached to the benzene ring of the alkylbenzene compound. When two or more alkyl groups are attached to the benzene ring of the alkylbenzene compound, the alkyl groups can be arranged at any suitable relative positions. For example, when two alkyl groups are attached to the benzene ring of the alkylbenzene compound, the second alkyl group may be at the ortho-, para-, or meta-position relative to the first alkyl group.

Optionally, the alkylbenzene compound or each alkylbenzene compound in the liquid product has a structure of Formula (I).

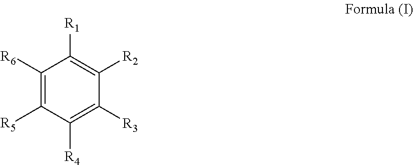

wherein R₁-R₆ are independently hydrogen or an alkyl group (such as a C₁-C₂₀ alkyl group, a C₁-C₁₅ alkyl group, a C₁-C₁₂ alkyl group, a C₁-C₁₀ alkyl group, or a C₁-C₅ alkyl group), and wherein at least one of R₁-R₆ is an alkyl group.

Optionally, at least one of R₁-R₆ of Formula (I), such as one, two, three, four, five, or six of R₁-R₆ of Formula (I), is/are a linear C₁-C₂₀ alkyl group, a branched C₄-C₂₀ alkyl group, a cyclic C₃-C₂₀ alkyl group, a linear C₁-C₁₅ alkyl group, a branched C₄-C₁₅ alkyl group, a cyclic C₃-C₁₅ alkyl group, a linear C₁-C₁₂ alkyl group, a branched C₄-C₁₂ alkyl group, a cyclic C₁-C₁₂ alkyl group, a linear C₁-C₁₀ alkyl group, a branched C₄-C₁₀ alkyl group, a cyclic C₃-C₁₀ alkyl group, a linear C₁-C₅ alkyl group, a branched C₄-C₅ alkyl group, a cyclic C₃-C₈ alkyl group, a linear C₁-C₆ alkyl group, a branched C₄-C₆ alkyl group, a cyclic C₃-C₆ alkyl group, a linear C₁-C₅ alkyl group, a branched C₄-C₅ alkyl group, or a cyclic C₃-C₅ alkyl group, such as a linear C₁-C₂₀ alkyl group, a linear C₁-C₁₅ alkyl group, a linear C₁-C₁₂ alkyl group, a linear C₁-C₁₀ alkyl group, a linear C₁-C₅ alkyl group, a linear C₁-C₆ alkyl group, or a linear C₁-C₅ alkyl group.

Optionally, at least one of R₁-R₆ of Formula (I), such as two, three, or four of R₁-R₆ of Formula (I), is/are a linear C₁-C₂₀ alkyl group, a branched C₄-C₂₀ alkyl group, a cyclic C₃-C₂₀ alkyl group, a linear C₁-C₁₅ alkyl group, a branched C₄-C₁₅ alkyl group, a cyclic C₃-C₁₅ alkyl group, a linear C₁-C₁₂ alkyl group, a branched C₄-C₁₂ alkyl group, a cyclic C₁-C₁₂ alkyl group, a linear C₁-C₁₀ alkyl group, a branched C₄-C₁₀ alkyl group, a cyclic C₃-C₁₀ alkyl group, a linear C₁-C₅ alkyl group, a branched C₄-C₅ alkyl group, a cyclic C₃-C₅ alkyl group, a linear C₁-C₆ alkyl group, a branched C₄-C₆ alkyl group, a cyclic C₃-C₆ alkyl group, a linear C₁-C₈alkyl group, a branched C₄-C₅ alkyl group, or a cyclic C₃-C₅ alkyl group, such as a linear C₁-C₂₀ alkyl group, a linear C₁-C₁₅ alkyl group, a linear C₁-C₁₂ alkyl group, a linear C₁-C₁₀ alkyl group, a linear C₁-C₈alkyl group, a linear C₁-C₆ alkyl group, or a linear C₁-C₈alkyl group.

For example, at least one of R₁-R₆ of Formula (I), such as one, two, three, four, five, or six of R₁-R₆ of Formula (I), are independently —CH₂R, wherein R is hydrogen, a linear C₁-C₁₅ alkyl group, a branched C₄-C₁₅ alkyl group, a cyclic C₃-C₁₅ alkyl group, a linear C₁-C₁₂ alkyl group, a branched C₄-C₁₂ alkyl group, a cyclic C₁-C₁₂ alkyl group, a linear C₁-C₁₀ alkyl group, a branched C₄-C₁₀ alkyl group, a cyclic C₃-C₁₀ alkyl group, a linear C₁-C₈alkyl group, a branched C₄-C₅ alkyl group, a cyclic C₃-C₅ alkyl group, a linear C₁-C₆ alkyl group, a branched C₄-C₆ alkyl group, a cyclic C₃-C₆ alkyl group, a linear C₁-C₅ alkyl group, a branched C₄-C₅ alkyl group, or a cyclic C₃-C₅ alkyl group, such as a linear C₁-C₁₅ alkyl group, a linear C₁-C₁₂ alkyl group, a linear C₁-C₁₀ alkyl group, a linear C₁-C₈alkyl group, a linear C₁-C₆ alkyl group, or a linear C₁-C₅ alkyl group.

For example, at least one of R₁-R₆ of Formula (I), such as two, three, or four of R₁-R₆ of Formula (I), are independently —CH₂R, wherein R is hydrogen, a linear C₁-C₁₅ alkyl group, a branched C₄-C₁₅ alkyl group, a cyclic C₃-C₁₅ alkyl group, a linear C₁-C₁₂ alkyl group, a branched C₄-C₁₂ alkyl group, a cyclic C₁-C₁₂ alkyl group, a linear C₁-C₁₀ alkyl group, a branched C₄-C₁₀ alkyl group, a cyclic C₃-C₁₀ alkyl group, a linear C₁-C₈alkyl group, a branched C₄-C₅ alkyl group, a cyclic C₃-C₅ alkyl group, a linear C₁-C₆ alkyl group, a branched C₄-C₆ alkyl group, a cyclic C₃-C₆ alkyl group, a linear C₁-C₅ alkyl group, a branched C₄-C₅ alkyl group, or a cyclic C₃-C₅ alkyl group, such as a linear C₁-C₁₅ alkyl group, a linear C₁-C₁₂ alkyl group, a linear C₁-C₁₀ alkyl group, a linear C₁-C₈alkyl group, a linear C₁-C₆ alkyl group, or a linear C₁-C₅ alkyl group.

Optionally, the alkylbenzene compound or each alkylbenzene compound in the liquid product has a structure of:

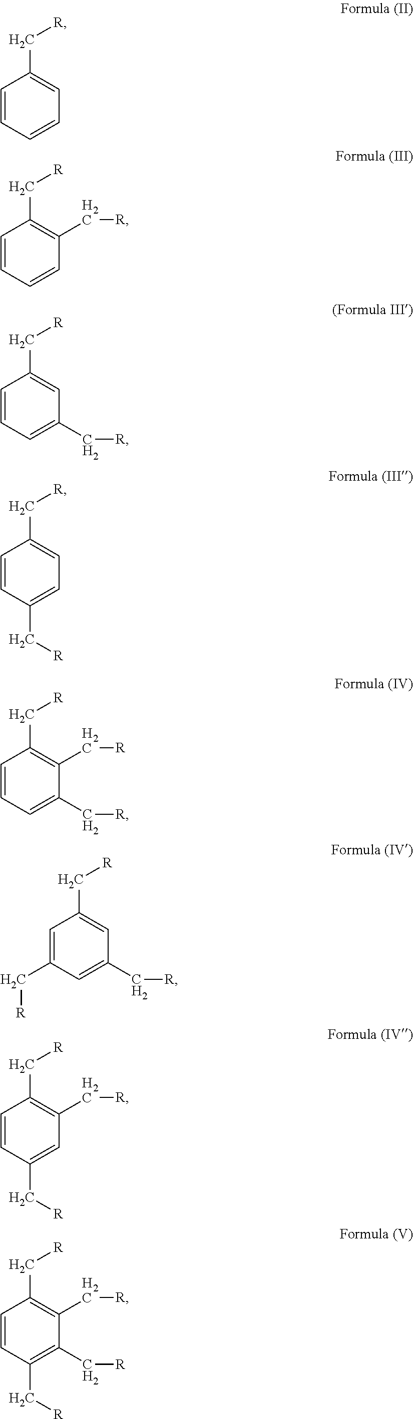

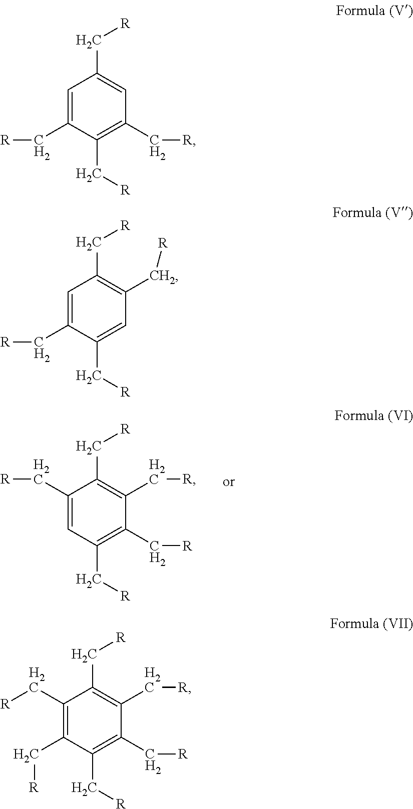

where each R is independently hydrogen, a C₁-C₁₅ alkyl group, a C₁-C₁₂ alkyl group, a C₁-C₁₀ alkyl group, or a C₁-C₅ alkyl group, such as a linear C₁-C₁₅ alkyl group, a branched C₄-C₁₅ alkyl group, a cyclic C₃-C₁₅ alkyl group, a linear C₁-C₁₂ alkyl group, a branched C₄-C₁₂ alkyl group, a cyclic C₁-C₁₂ alkyl group, a linear C₁-C₁₀ alkyl group, a branched C₄-C₁₀ alkyl group, a cyclic C₃-C₁₀ alkyl group, a linear C₁-C₅ alkyl group, a branched C₄-C₅ alkyl group, a cyclic C₃-C₅ alkyl group, a linear C₁-C₆ alkyl group, a branched C₄-C₆ alkyl group, a cyclic C₃-C₆ alkyl group, a linear C₁-C₅ alkyl group, a branched C₄-C₅ alkyl group, or a cyclic C₃-C₅ alkyl group, such as a linear C₁-C₁₅ alkyl group, a linear C₁-C₁₂ alkyl group, a linear C₁-C₁₀ alkyl group, a linear C₁-C₅ alkyl group, a linear C₁-C₆ alkyl group, or a linear C₁-C₅ alkyl group, for example, a linear C₁-C₁₅ alkyl group, a linear C₁-C₁₂ alkyl group, a linear C₁-C₁₀ alkyl group, a linear C₁-C₅ alkyl group, a linear C₁-C₆ alkyl group, or a linear C₁-C₈alkyl group.

For example, the alkylbenzene compound or each alkylbenzene compound in the liquid product has the structure of Formula (III), (III′), (III″), (IV), (IV′), (IV″), (V), (V′), or (V″), where R is as defined above. For example, the alkylbenzene compound or each alkylbenzene compound in the liquid product has the structure of Formula (II), (III), (III′), (III″), (IV), (IV′), (IV″), (V), (V′), or (V″), where R is as defined above. For example, the alkylbenzene compound or each alkylbenzene compound in the liquid product has the structure of Formula (II), (III), (III′), (III″), (IV), (IV′), or (IV″), where R is as defined above.

In some forms, the alkylbenzene compound or each alkylbenzene compound in the liquid product has the structure of Formula (III), (III′), (III″), (IV), (IV′), (IV″), (V), (V′), (V″), or (VI), where two R groups are independently a linear C₃-C₁₅ alkyl group, a linear C₃-C₁₂ alkyl group, a linear C₃-C₁₀ alkyl group, a linear C₃-C₅ alkyl group, a linear C₄-C₁₅ alkyl group, a linear C₄-C₁₂ alkyl group, a linear C₄-C₁₀ alkyl group, or a linear C₄-C₅ alkyl group, such as a linear C₄ or a linear C₅ alkyl group; and the other R group(s) is/are hydrogen. In some forms, the product containing alkylbenzene compounds having such structures may be more biodegradable compared to alkylbenzene compounds having a single, long, branched alkyl chain attached to the benzene ring, such as

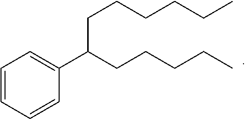 [SMILES: CCCCCCC(CCCCC)c1ccccc1]

In some forms, the alkylbenzene compound or each alkylbenzene compound in the liquid product has the structure of Formula (III), (III′), (III″), (IV), (IV′), or (IV″), where two R groups are independently a linear C₃-C₁₅ alkyl group, a linear C₃-C₁₂ alkyl group, a linear C₃-C₁₀ alkyl group, a linear C₃-C₅ alkyl group, a linear C₄-C₁₅ alkyl group, a linear C₄-C₁₂ alkyl group, a linear C₄-C₁₀ alkyl group, or a linear C₄-C₅ alkyl group, such as a linear C₄ or a linear C₅ alkyl group; and the other R is hydrogen.

In some forms, the liquid product can contain an alkylbenzene compound having the structure of Formula (III), (III′), or (III″), where each R is independently a linear C₃-C₁₅ alkyl group, a linear C₃-C₁₂ alkyl group, a linear C₃-C₁₀ alkyl group, a linear C₃-C₅ alkyl group, a linear C₄-C₁₅ alkyl group, a linear C₄-C₁₂ alkyl group, a linear C₄-C₁₀ alkyl group, or a linear C₄-C₅ alkyl group, such as a linear C₄ or a linear C₅ alkyl group.

In some forms, the liquid product can contain an alkylbenzene compound having the structure of Formula (IV), (IV′), or (IV″), where two R groups are independently a linear C₃-C₁₅ alkyl group, a linear C₃-C₁₂ alkyl group, a linear C₃-C₁₀ alkyl group, a linear C₃-C₅ alkyl group, a linear C₄-C₁₅ alkyl group, a linear C₄-C₁₂ alkyl group, a linear C₄-C₁₀ alkyl group, or a linear C₄-C₅ alkyl group, such as a linear C₄ or a linear C₅ alkyl group; and the other R is hydrogen.

In some forms, the liquid product can contain an alkylbenzene compound having the structure of

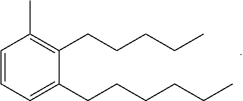 [SMILES: CCCCCCc1cccc(C)c1CCCCC]

b. Other Compounds

Optionally, the liquid product further contains one or more other aromatic compounds that are not alkylbenzene compounds (such as one or more alkylpolyaromatic compounds and/or one or more polyaromatic compounds), one or more alkanes, and/or one or more non-aromatic unsaturated compounds (such as olefins). Additionally or alternatively, in addition to the liquid, the product further contains a wax. The wax typically contains long chain alkanes, i.e., C41-C100 alkanes. The presence of wax in the product typically indicates that the depolymerization reaction is not completed and can further proceed for a longer period of time to convert the wax to liquid.

The alkanes in the liquid product typically have a carbon number in a range from 7 to 40. The alkylpolyaromatic compounds and polyaromatic compounds can each contain two aromatic rings, three aromatic rings, four aromatic rings, five aromatic rings, or six aromatic rings. For example, the alkylpolyaromatic compound in the liquid product is alkylnaphthalene, alkylanthracene, or alkylphenanthrene, or a combination thereof. Optionally, the alkylpolyaromatic compound in the liquid product is a partially hydrogenated analog of a polycyclic aromatic compound. For example, the alkylpolyaromatic compound in the liquid product is a partially hydrogenated analog of alkylnaphthalene, i.e., alkyltetralin. The non-aromatic unsaturated compound in the liquid product can be an olefin. Olefins are unsaturated hydrocarbons that contain at least one carbon-carbon double bond. The olefin in the product can be linear, branched, or cyclic.

For example, in addition to the alkylbenzene compound(s), the product further contains one or more polyaromatic compounds (e.g. naphthalene, anthracene, phenanthrene, or a partially hydrogenated analog thereof, such as tetralin, or a combination thereof), one or more alkylpolyaromatic compounds (e.g. alkylnaphthalene, alkylanthracene, alkylphenanthrene, or a partially hydrogenated analog thereof, such as alkyltetralin, or a combination thereof), one or more C7-C40 alkanes, and/or one or more olefins (e.g., a diene and/or a cycloalkene).

The other aromatic compounds and non-aromatic unsaturated compounds can contain any number of carbons described above for the alkylbenzene compound. The other aromatic compounds and non-aromatic unsaturated compounds can have a Mw or Mn as described above for the alkylbenzene compound.

The other aromatic compound(s), alkane(s), and/or non-aromatic unsaturated compounds in the liquid product, and/or the long-chain alkanes in the wax, when present, can be separated from the alkylbenzene compounds in the liquid product. For example, after exiting the reactor, the product stream 110 containing a liquid and optionally a wax can be fed into a separation unit. For example, as shown in FIG. 12, the separation unit 20 separates the liquid and optionally wax product stream 110 to produce a C₆-C₁₀ alkane and C₆-C₁₀ aromatic compound stream 111, an alkane stream 112 (e.g., a mixture of alkanes, such as alkanes with carbon numbers ranging from 11 to 80 or from 16 to 22), and an alkylaromatic stream 113. The alkylaromatic stream 113 may contain predominantly alkylbenzene compound(s) or a mixture of alkylbenzene compound(s) and alkylpolyaromatic compound(s) that may be subjected to further separation to produce an alkylbenzene stream.

### 4. Gas

The process described herein may also produce other compounds that are not in the form of a liquid or wax. For example, depolymerization of the solid waste using the process described herein may also produce a gas. Generally, the gas contains a saturated or unsaturated compound, optionally more than one saturated or unsaturated compound, such as a saturated C₁-C₆ alkane, hydrogen, benzene, or toluene, or a combination thereof.

Optionally, the saturated C₁-C₆ alkane in the produced gas can be linear, branched, or cyclic. For example, the saturated alkane is a linear C₁-C₆ saturated alkane, a branched C₄-Cₑ saturated alkane, or a cyclic C₃-C₆ saturated alkane, optionally a linear C₁-C₆ saturated alkane, a branched C₄-C₆ saturated alkane, or a cyclic C₃-C₆ saturated alkane.

For example, the gas produced from depolymerization of the solid waste contains C₁-C₆ saturated alkanes, such as C₃H₈, norm-butane, iso-butane, norm-pentane, and/or pentane isomers (such as isopentane, neopentane).

### 5. Operating Conditions

The reactor is operated at a suitable temperature, under a suitable H₂ pressure, and for a period of time sufficient to convert the solid hydrocarbon polymer of the waste material to the product containing a liquid and optionally a wax. The liquid contains one or more alkylbenzene compounds. Optionally, the liquid product further contains other aromatic compounds that are not alkylbenzene compounds, such as one or more alkylpolyaromatic compounds and/or polyaromatic compounds. When other aromatic compounds are present in the liquid product, the mass selectivity of the alkylbenzene compound(s) relative to all aromatic compounds in the liquid is at least 25%, at least 30%, at least 50%, in a range from 25% to 99.99%, from 25% to 99.9%, from 25% to 99%, from 25% to 95%, from 25% to 90%, from 25% to 90%, from 25% to 80%, from 25% to 70%, or from 25% to 60%.

a. Temperature, Pressure, and Period of time

Typically, the operating temperature for the reactor is relatively low, for example, the operating temperature does not exceed 500 °C. Such low operating temperatures allow for energy-efficient depolymerization of waste hydrocarbon polymers.

Optionally, the temperature sufficient to convert the waste hydrocarbon polymer to the product that contains a liquid containing one or more alkylbenzene compounds is up to 500 °C, up to 450 °C, up to 400 °C, up to 350 °C, up to 320 °C, up to 300 °C, up to 290 °C, between 250 °C and 500 °C, between 250 °C and 450 °C, between 250 °C and 400 °C, between 300 °C and 500 °C, between 320 °C and 500 °C, between 300 °C and 450 °C, between 320 °C and 450 °C, between 300 °C and 400 °C, between 300 °C and 360 °C, between 250 °C and 350 °C, between 250 °C and 350 °C, between 250 °C and 320 °C, or between 250 °C and 300 °C, such as about 280 °C.

The temperature is selected based on the hydrocarbon polymer(s) in the waste material, the catalyst, and/or the H₂ pressure under which the reactor is operated. For example, when a Pt/silica-Al₂O₃ catalyst is used for depolymerization of the waste material, the temperature sufficient to convert the hydrocarbon polymer to the product is up to 350 °C, up to 320 °C, up to 300 °C, up to 290 °C, between 250 °C and 350 °C, between 250 °C and 320 °C, or between 250 °C and 300 °C, such as about 280 °C. For example, when a Pt/silica-Al₂O₃ catalyst is used for depolymerization of polyethylene, the temperature sufficient to convert the hydrocarbon polymer to the product is up to 350 °C, up to 320 °C, up to 300 °C, up to 290 °C, between 250 °C and 350 °C, between 250 °C and 320 °C, or between 250 °C and 300 °C, such as about 280 °C.

Typically, the H₂ pressure under which the reactor is operated for depolymerization of the plastic waste material is effective to produce the liquid product containing alkylbenzene compound(s) with an alkylbenzene mass selectivity of at least 25% relative to all aromatic compounds in the liquid product.

For example, when the reactor operates under a constant H₂ pressure to form the liquid product, the P(H₂) in the reactor can be in a range from 1 atm to 8 atm, from 1 atm to 7 atm, from 1 atm to 6 atm, from 1 atm to 5 atm, or from 1 atm to 4 atm to depolymerize a plastic waste material contained therein, and the liquid product obtained from the depolymerization reaction contains alkylbenzene compound(s) with a mass selectivity of at least 25% relative to all aromatic compounds in the liquid product.

When the P(H₂) under which the reactor is operated to form the liquid product is varied from a first P(H₂) to a second P(H₂), the first P(H₂) can be effective to depolymerize the plastic waste material contained therein and form fragments of hydrocarbon polymer, and the second P(H₂) can be effective to form the liquid product that contains alkylbenzene compound(s) with a mass selectivity of at least 25% relative to all aromatic compounds in the liquid product. Typically, the second P(H₂) is lower than the first P(H₂). In these forms, operating the reactor under the first high P(H₂) can break the long chain of the hydrocarbon polymer of the waste and form fragments of the hydrocarbon polymer having lower weight average molecular weight (Mw). Generally, when the fragments of the hydrocarbon polymer obtained under the first P(H₂) reach an average chain length of <100 carbon atoms, the P(H₂) in the reactor can be adjusted to the second, lower P(H₂). Operating the reactor under the second, lower P(H₂) can form the liquid product containing alkylbenzene compound(s). In some forms, the first P(H₂) can range from 5 atm to 20 atm, from 5 atm to 18 atm, from 5 atm to 15 atm, from 5 atm to 12 atm, from 5 atm to 10 atm, such as about 8 atm, and the second P(H₂) can range from 0 to <5 atm, from 0 to 4 atm, from 0 to 3 atm, or from 0 to 2 atm. In some forms, the second P(H₂) can be 0, such that the reactor is operated under an inert gas pressure, for example, an argon pressure ranging from 0 to <5 atm, from 0 to 4 atm, from 0 to 3 atm, or from 0 to 2 atm.

When the P(H₂) under which the reactor is operated to form the liquid product is varied from a first P(H₂) to a second P(H₂) then to a third P(H₂). Typically, the second P(H₂) is lower than the first P(H₂), such as those described above, and the third P(H₂) is higher than the second P(H₂). In these forms, the first high P(H₂) can break the long chain of the hydrocarbon polymer of the waste and form fragments of the hydrocarbon polymer having lower weight average molecular weight (Mw); the second lower P(H₂) can form a liquid containing aromatic compounds (which include alkylbenzenes, polyaromatics, etc.); and the third P(H₂) can form the liquid product that contains alkylbenzene compound(s) with a mass selectivity of at least 25% relative to all aromatic compounds in the liquid product. In these forms, the first P(H₂) in the reactor can be adjusted to the lower, second P(H₂) when the fragments of the hydrocarbon polymer obtained under the first P(H₂) reach an average chain length of <100 carbon atoms; and the second P(H₂) in the reactor can be adjusted to the higher, third P(H₂) when the total yield of the aromatic compounds reaches a plateau, such as determined using ¹H NMR spectroscopy combined with gas chromatography. Operating the reactor under the third P(H₂) can suppress the formation of polyaromatics and further increase the formation of alkylbenzene compounds. In some forms, the first P(H₂) can range from 5 atm to 20 atm, from 5 atm to 18 atm, from 5 atm to 15 atm, from 5 atm to 12 atm, from 5 atm to 10 atm, such as about 8 atm, the second P(H₂) can range from 0 to <5 atm, from 0 to 4 atm, from 0 to 3 atm, or from 0 to 2 atm, and the third P(H₂) can range from 5 atm to 15 atm, from 5 atm to 12 atm, or from 5 atm to 10 atm. In some forms, the second P(H₂) can be 0, such that the reactor is operated under an inert gas pressure, for example, an argon pressure ranging from 0 to <5 atm, from 0 to 4 atm, from 0 to 3 atm, or from 0 to 2 atm.

Typically, the period of time sufficient to convert the hydrocarbon polymer to the product is up to 24 hours, up to 6 hours, up to 5.5 hours, up to 5 hours, up to 4.5 hours, up to 4 hours, up to 3.5 hours, up to 3 hours, up to 2.5 hours, up to 2 hours, up to 1.5 hours, up to 1 hour, up to 55 minutes, up to 50 minutes, up to 45 minutes, up to 40 minutes, up to 35 minutes, up to 30 minutes, up to 25 minutes, up to 20 minutes, up to 15 minutes, up to 10 minutes, up to 5 minutes, in a range from 5 minutes to 6 hours, from 5 minutes to 5.5 hours, from 5 minutes to 5 hours, from 5 minutes to 4.5 hours, from 5 minutes to 4 hours, from 5 minutes to 3.5 hours, from 5 minutes to 3 hours, from 5 minutes to 2.5 hours, from 5 minutes to 2 hours, from 5 minutes to 1.5 hours, from 1 minute to 1 hour, from 5 minutes to 1 hour, from 10 minutes to 1 hour, from 15 minutes to 1 hour, from 20 minutes to 1 hour, from 30 minutes to 1 hour, from 1 minute to 55 minutes, from 1 minute to 50 minutes, from 1 minute to 45 minutes, from 1 minute to 40 minutes, from 1 minute to 35 minutes, or from 1 minute to 30 minutes. When the hydrogen pressure in the reactor is varied, the reactor can be operated under each hydrogen pressure for any of the time periods described above.

For example, the depolymerization reaction can be performed at a suitable temperature, such as up to 500 °C, up to 450 °C, up to 400 °C, up to 380 °C, up to 350 °C, up to 320 °C, up to 300 °C, up to 290 °C, between 250 °C and 500 °C, between 250 °C and 450 °C, between 250 °C and 400 °C, between 320 °C and 500 °C, between 320 °C and 450 °C, between 320 °C and 400 °C, between 350 °C and 500 °C, between 350 °C and 450 °C, between 350 °C and 400 °C, between 250 °C and 360 °C, between 250 °C and 350 °C, between 250 °C and 330 °C, between 250 °C and 320 °C, or between 250 °C and 300 °C, such as about 280 °C. When the hydrogen pressure in the reactor is varied, the reactor can be operated under each hydrogen pressure at any of the temperatures described above.

For example, the depolymerization reaction is performed at any one of the temperatures above, under a constant H₂ pressure, such as a constant H₂ pressure in a range from 1 atm to 8 atm, from 1 atm to 7 atm, from 1 atm to 6 atm, from 1 atm to 5 atm, or from 1 atm to 4 atm, for a suitable period of time, such as from 5 minutes to 6 hours, from 10 minutes to 6 hours, from 20 minutes to 6 hours, from 30 minutes to 6 hours, from 5 minutes to 5 hours, from 10 minutes to 5 hours, from 20 minutes to 5 hours, from 30 minutes to 5 hours, from 5 minutes to 4 hours, from 10 minutes to 4 hours, from 20 minutes to 4 hours, from 30 minutes to 4 hours, from 5 minutes to 3 hours, from 10 minutes to 3 hours, from 20 minutes to 3 hours, from 30 minutes to 3 hours, from 5 minutes to 2 hours, from 10 minutes to 2 hours, from 20 minutes to 2 hours, or from 30 minutes to 2 hours that are sufficient to convert the hydrocarbon polymer to the product that contains a liquid containing one or more alkylbenzene compounds.

### 6. Characterization of the Depolymerization Process

The disclosed process for depolymerizing a solid waste containing hydrocarbon polymer(s) can be characterized by alkylbenzene yield and alkylbenzene mass selectivity.

a. Yield

Typically, the liquid product produced in the depolymerization reaction in step (ii) contains alkylbenzene compound(s) with a yield of at least 4% or at least 5%, by weight. The yield of alkylbenzene compound(s) is calculated as the sum of the weights of the alkylbenzene compound(s) in the liquid product divided by the sum of the weights of the hydrocarbon polymer(s) of the waste material, as a percentage.

b. Selectivity

The process disclosed herein may produce other compounds in the liquid product that are not alkylbenzene compounds, such as other aromatic compounds (e.g., alkylpolyaromatic compounds and/or polyaromatic compounds), non-aromatic unsaturated compounds, and/or alkanes. For example, depolymerization of polyethylene using the disclosed process produces alkylbenzene compound(s), such as one or more alkylbenzene compounds as described above, and one or more alkylpolyaromatic compounds such as those described above, in the liquid product.

The mass selectivity of the disclosed process to convert the hydrocarbon polymer to the alkylbenzene compound(s) relative to all aromatic compounds in the liquid product can be measured by NMR in combination with GPC or GC-FID. Exemplary methods for determining the selectivity of the alkylbenzene compound(s) relative to all aromatic compounds in the liquid product, are shown in Example 1.

Typically, the disclosed process is able to convert the hydrocarbon polymer to the liquid product with an alkylbenzene mass selectivity of at least 25%, at least 30%, at least 50%, in a range from 25% to 99.99%, from 25% to 99.9%, from 25% to 99%, from 25% to 95%, from 25% to 90%, from 25% to 90%, from 25% to 80%, from 25% to 70%, or from 25% to 60%, relative to all aromatic compounds in the liquid product.

For example, as shown in FIG. 12, the mass selectivity of the alkylbenzene compound(s) in the liquid of product stream 110 can be at least 25%, at least 30%, at least 50%, in a range from 25% to 99.99%, from 25% to 99.9%, from 25% to 99%, from 25% to 95%, from 25% to 90%, from 25% to 90%, from 25% to 80%, from 25% to 70%, or from 25% to 60%, relative to all aromatic compounds in the liquid of product stream 110.

### C. Optional Steps

### 1. Processing the Waste Hydrocarbon Polymer

The process optionally includes a step of processing the waste material containing one or more hydrocarbon polymers to a suitable form prior to step (i) or subsequent to step (i) and prior to step (ii). Generally, the waste material containing one or more hydrocarbon polymer(s) is processed to a form that improves the contact between the polymer and the catalyst.

For example, the waste material is in the form of a solid and the solid waste is processed by shredding, cutting, and/or grinding the waste hydrocarbon polymer to small parts prior to feeding it into the reactor, such that the contact between processed solid waste and the catalyst is improved compared with the solid waste prior to processing.

Optionally, the waste material is in the form of a solid and the solid waste is dissolved in a solvent, optionally in a hydrocarbon solvent, prior to feeding it into the reactor or the waste is dissolved in the solvent in the reactor after feeding it into the reactor and prior to the depolymerization reaction, such that the contact between the dissolved solid waste and the catalyst is improved compared with the solid waste without dissolving. For example, the solvent is fed into the reactor, and the solid waste is subsequently fed into the reactor and dissolved in the solvent. Alternatively, the solid waste is fed into the reactor and the solvent is subsequently fed into the reactor and dissolves the solid waste. Optionally, the solid waste and the solvent are fed into the reactor simultaneously or substantially simultaneously, and the solid waste is dissolved in the solvent in the reactor.

Typically, the hydrocarbon solvent is a small hydrocarbon solvent, i.e., a hydrocarbon solvent containing less than 11 carbon atoms. Suitable hydrocarbon solvents for dissolving the solid waste include, but are not limited to, hexane, cyclohexane, isopentane, a mixture of n-pentane and isopentane (e.g. pentane 60/40 and pentane 80/20), toluene, TOPSol (e.g. TOPSol 60/145, TOPSol A100, TOPSol A150, TOPSol A150ND, TOPSol BF, TOPSol X2000, and TOPSol 2046), a mixture of paraffins, cycliparaffins, and aromatics (WS 200), xylene, benzene, ethylbenzene, and a mixture of xylene and ethylbenzene.

For example, as shown in FIG. 12, a solvent can be fed into the reactor 10 via a reagent stream 200 prior to feeding the waste material, or subsequent to feeding the waste material and prior to the depolymerization reaction, or simultaneously or substantially simultaneously with feeding the waste material, such that the solid waste is dissolved in the solvent in the reactor.

### 2. Feeding Hydrogen Gas

Optionally, the process includes, during step (ii), a step of feeding hydrogen gas into the reactor. The step of feeding H₂ gas may occur one or more times during the depolymerization reaction in step (ii) to maintain a sufficient H₂ pressure in the reactor for achieving an alkylbenzene mass selectivity of at least 25% relative to all aromatic compounds in the liquid of the product.

Optionally, the process includes, after step (ii), a step of removing hydrogen gas and optionally hydrocarbon gas from the reactor and then feeding hydrogen gas into the reactor to achieve a second value for P(H₂) in the reactor that is different from the P(H₂) in step (ii). Subsequent to the feeding of hydrogen gas, the process may include a step of (d) operating the reactor at a suitable temperature, under the second P(H₂), and for a sufficient period of time to convert any unreacted hydrocarbon polymer to a second product. The second product may contain a liquid and/or a wax, and the liquid may contain one or more alkylbenzene compounds, such as those described above. The specific temperature and time period for the subsequent depolymerization reaction performed under the second P(H₂) depends on the specific catalyst and/or plastic waste material used in the reaction, and may be the same or different from those used in step (ii). In the depolymerization reaction performed under the second P(H₂) in step (d) (that is different from the P(H₂) used in step (ii)), the pressure in the reactor can range from 0.01 atm to 50 atm, from 0.1 atm to 50 atm, from 0.5 atm to 40 atm, from 0.1 atm to 40 atm, from 0.1 atm to 30 atm, from 0.1 atm to 20 atm, from 0.1 atm to 10 atm, from 0.1 atm to 5 atm, or from 0.5 atm to 5 atm, such as from 1 atm to 2 atm.

### 3. Cooling the Reactor to Room Temperature

The process optionally includes a step of cooling the reactor to a temperature below 50 °C at 1 atm, such as to room temperature, after the depolymerization reaction in step (ii) or optional step (d). The reactor may be cooled by any suitable method. For example, the reactor can be cooled in an airflow or in a water bath of room temperature (i.e., 22 °C-25 °C at 1 atm).

### 4. Recycling the Catalyst

The method may include a step of recycling the catalyst after the depolymerization reaction in step (ii) or optional step (d). Typically, the catalyst can be recycled and reused for the depolymerization reaction without significant loss of activity. For example, the catalyst can be recycled and reused for the depolymerization reaction with less than 5%, less than 10%, or less than 20% decrease in alkylbenzene yield. Optionally, the selectivity of the process to form alkylbenzene compounds relative to all aromatic compounds using a recycled catalyst is the same or substantially the same as that using a fresh catalyst. The catalyst can be recycled and reused at least 2 times, at least 3 times, at least 4 times, at least 5 times, at least 6 times, at least 7 times, at least 8 times, at least 9 times, or at least 10 times.

Catalyst may be recycled by oxidation with an oxidizing gas and then optionally reduced with a reducing gas.

a. Oxidation

After the depolymerization reaction in step (ii) or optional step (d), the catalyst is recycled. For example, the catalyst is exposed to an oxidizing gas to oxidize the catalyst.

Examples of suitable oxidizing gas for oxidizing the catalyst include, but are not limited to, oxygen and air. For example, the oxidizing gas is oxygen.

Generally, the catalyst is oxidized with an oxidizing gas at a temperature at least 200 °C, at least 250 °C, at least 300 °C, up to 450 °C, up to 400 °C, from 200 °C to 450 °C, from 250 °C to 450 °C, or from 300 °C to 450 °C, such as from 350 °C to 450 °C.

Generally, the catalyst is oxidized with the oxidizing gas for a time period at least 10 minutes, at least 20 minutes, at least 30 minutes, at least 40 minutes, at least 50 minutes, at least 1 hour, up to 5 hours, up to 4 hours, up to 3 hours, up to 2 hours, between 10 minutes and 2 hours, between 30 minutes and 2 hours, or between 1 hour and 2 hours.

For example, the oxidation is performed at a temperature at least 200 °C, at least 250 °C, at least 300 °C, up to 450 °C, up to 400 °C, from 200 °C to 450 °C, from 250 °C to 450 °C, or from 300 °C to 450 °C, for a time period at least 10 minutes, at least 20 minutes, at least 30 minutes, at least 40 minutes, at least 50 minutes, at least 1 hour, up to 5 hours, up to 4 hours, up to 3 hours, up to 2 hours, between 10 minutes and 2 hours, between 30 minutes and 2 hours, or between 1 hour and 2 hours. For example, the oxidation is performed at a temperature from 250 °C to 450 °C for a time period between 10 minutes and 2 hours or between 30 minutes and 2 hours.

b. Reduction

Optionally, after oxidation the oxidized catalyst is reduced with a reducing gas. Examples of suitable reducing gas for reducing the oxidized catalyst include, but are not limited to, hydrogen, carbon monoxide, ammonia, methane, and nitric oxide. For example, the reducing gas is hydrogen.

The reduction temperature can be at least 150 °C, at least 200 °C, at least 250 °C, up to 400 °C, up to 350 °C, up to 300 °C, between 150 °C and 400 °C, between 150 °C and 350 °C, or between 150 °C and 300 °C, such as between 200 °C and 300 °C. Typically, the reduction temperature is lower than the oxidation temperature. For example, the catalyst is oxidized at 400 °C and then reduced at 250 °C.

The period of time for the reduction step can be the same, substantially the same, or different than the period of time for the oxidation step.

For example, the reduction step is performed at a temperature at least 150 °C, at least 200 °C, at least 250 °C, up to 400 °C, up to 350 °C, up to 300 °C, between 150 °C and 400 °C, between 150 °C and 350 °C, or between 150 °C and 300 °C for a period of time at least 10 minutes, at least 20 minutes, at least 30 minutes, at least 40 minutes, at least 50 minutes, at least 1 hour, up to 5 hours, up to 4 hours, up to 3 hours, up to 2 hours, between 10 minutes and 2 hours, between 30 minutes and 2 hours, or between 1 hour and 2 hours. For example, the oxidation is performed at a temperature from 250 °C to 450 °C for a time period between 10 minutes and 2 hours or between 30 minutes and 2 hours, and the reduction is performed at a temperature between 200 °C and 300 °C for a time period between 30 minutes and 3 hours or between 1 hour and 3 hours.

Optionally, the period of time for the reduction step is different than the period of time for the oxidation step. The period of time for the reduction step may be longer or shorter than the period of time for the oxidation step. For example, the period of time for the oxidation step is 2 hours, and the period of time for the reduction step is 3 hours.

### 7. Separating the Liquid and/or Wax Product

When the product formed in step (ii) contains a liquid and a wax, the upcycling process can include a step of separating the liquid and wax in the product and optionally further separates the compounds in the liquid product subsequent to step (ii). For example, as shown in FIG. 12, the product stream 110 containing a liquid and a wax enters into a separation unit 20 and is separated into two or more streams. The product stream 110 enters into separation unit 20 and is separated into three streams: an alkylaromatic compound stream 113, an alkane stream 112 (i.e., a mixture of alkanes, such as alkanes with carbon numbers ranging from 11 to 80 or from 16-22), and an C₆-C₁₀ alkane and C₆-C₁₀ aromatic stream 111.

Suitable separators that can be used for the step of separating the liquid and/or wax product are known. For example, the liquid and/or wax product can be separated by distillation as described in U.S. Pat. No. 2,848,387 to Glazier, et al. and U.S. Pat. No. 3,308,060 to Ellis.

As noted in Example 1 below, the wax generally contains a negligible amount of alkylaromatic compounds, such as less than 1% by weight.

### 8. Feeding Catalyst into the Reactor

The process optionally includes a step of feeding the catalyst into the reactor prior to step (i), or subsequent to step (i) and prior to step (ii), or simultaneously or substantially simultaneously with step (i). The catalyst in the reactor is in contact with the hydrocarbon polymer of the waste material. For example, as shown in FIG. 12, the catalyst is fed into the reactor 10 via a catalyst stream 200 prior to feeding the waste material, or subsequent to feeding the waste material and prior to the depolymerization reaction, or simultaneously or substantially simultaneously with feeding the waste material.

The disclosed processes and compositions can be further understood through the following enumerated paragraphs.

1. A process for upcycling a waste material, wherein the waste material comprises a hydrocarbon polymer, comprising
(i) feeding the waste material into a reactor,
   wherein the reactor comprises a catalyst therein,
      wherein the catalyst comprises a transition metal; and
(ii) operating the reactor at a sufficient temperature, under a sufficient hydrogen gas pressure (P(H₂)), and for a sufficient period of time to convert the hydrocarbon polymer to a product comprising a liquid, wherein the liquid comprises an alkylbenzene compound.

2. The process of paragraph 1, wherein the liquid further comprises one or more other aromatic compounds and optionally one or more alkane and/or one or more non-aromatic compounds.

3. The process of paragraph 2, wherein the mass selectivity of alkylbenzene compound relative to all aromatic compounds in the liquid is at least 25%, at least 30%, at least 50%, in a range from 25% to 99.99%, from 25% to 99.9%, from 25% to 99%, from 25% to 95%, from 25% to 90%, from 25% to 90%, from 25% to 80%, from 25% to 70%, or from 25% to 60%.

4. The process of any one of paragraphs 1-3, wherein the hydrocarbon polymer is polyethylene, polypropylene, polystyrene, a copolymer of polyethylene and polypropylene, or acrylonitrile butadiene styrene.

5. The process of any one of paragraphs 1-4, wherein the alkylbenzene compound contains from 7 to 40 carbon atoms or from 10 to 30 carbon atoms.

6. The process of any one of paragraphs 1-5, wherein the alkylbenzene compound has a weight average molecular weight (Mw) in a range from 150 g mol⁻¹ to 800 g mol⁻¹, from 200 g mol⁻¹ to 800 g mol⁻¹, from 200 g mol⁻¹ to 600 g mol⁻¹, from 200 g mol⁻¹ to 500 g mol⁻¹, or from 200 g mol⁻¹ to 400 g mol⁻¹.

7. The process of any one of paragraphs 1-6, wherein the alkylbenezene has a structure of Formula (I):

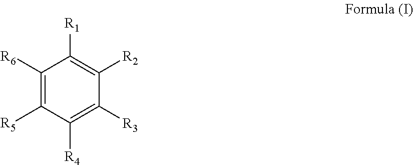

wherein R₁-R₆ are independently hydrogen, an alkyl group, and wherein at least one of R₁-R₆ is an alkyl group.

8. The process of paragraph 7, wherein R₁-R₆ are independently hydrogen or —CH₂R, wherein R is a hydrogen, C₁-C₁₉ alkyl group, C₁-C₁₅ alkyl group, a C₁-C₁₂ alkyl group, a C₁-C₁₀ alkyl group, or a C₁-C₅ alkyl group.

9. The process of any one of paragraphs 1-8, wherein the alkylbenzene has a structure of:

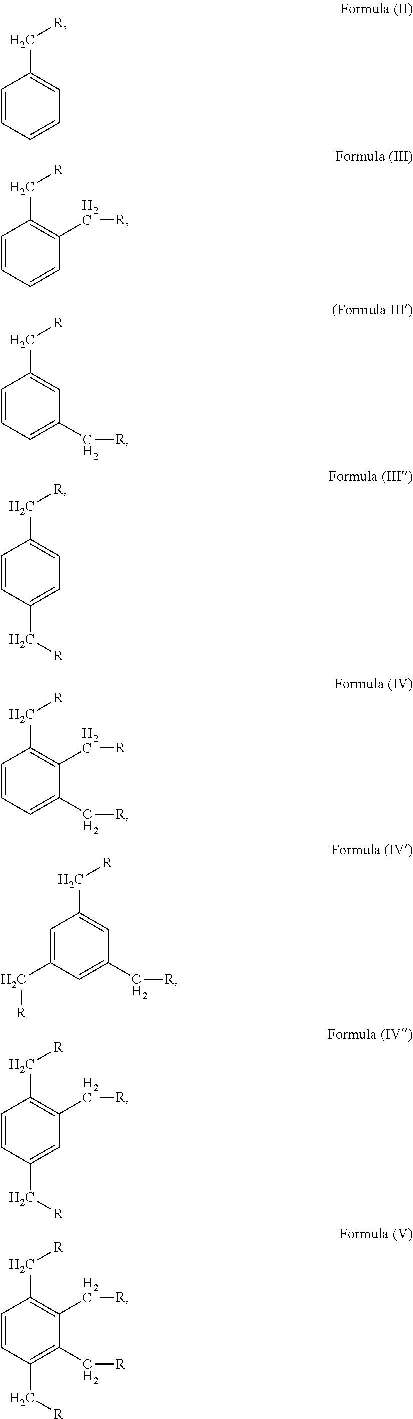

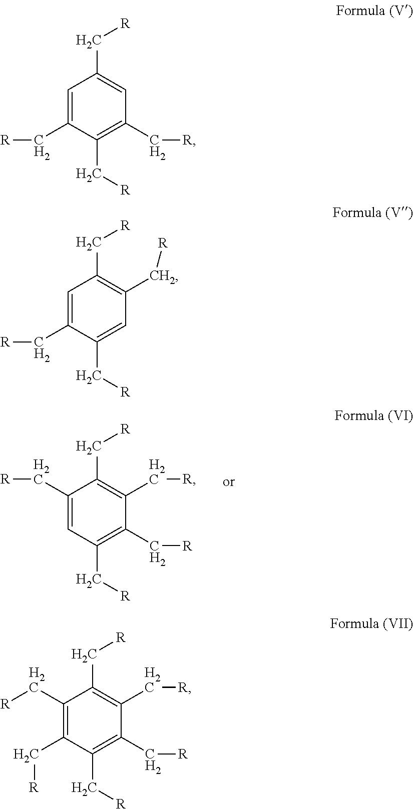

or a combination thereof,
wherein each R is independently hydrogen, a C₁-C₁₅ alkyl group, a C₁-C₁₂ alkyl group, a C₁-C₁₀ alkyl group, or a C₁-C₅ alkyl group.

10. The process of any one of paragraphs 1-9, wherein the transition metal is selected from the group consisting of platinum, palladium, ruthenium, iridium, rhenium, rhodium, iron, cobalt, nickel, copper, molybdenum, and tungsten.

11. The process of any one of paragraphs 1-10, wherein the catalyst comprises more than one transition metal, wherein each of the transition metals is selected from the group consisting of platinum, palladium, ruthenium, iridium, rhenium, rhodium, iron, cobalt, nickel, copper, molybdenum, and tungsten.

12. The process of any one of paragraphs 1-11, wherein the catalyst comprises more than one transition metal, wherein each of the transition metals is selected from the group consisting of platinum, palladium, ruthenium, iridium, rhodium, iron, cobalt, nickel, copper, molybdenum, and tungsten; and wherein the catalyst further comprises a second metal, wherein the second metal is different from each of the transition metals, and wherein the second metal is selected from the group consisting of rhenium, tin, lead, tungsten, molybdenum, chromium, manganese, and zinc, or a combination thereof

13. The process of any one of paragraphs 1-12, wherein the catalyst is a metal, a mixture of two or more metals comprising the transition metal, a metal oxide of the transition metal, or a metal carbide of the transition metal, or a combination thereof.

14. The process of any one of paragraphs 1-13, wherein the catalyst is dispersed on a surface of a first support and wherein the catalyst is in the form of atoms, nanoclusters, or nanoparticles, or a combination thereof.

15. The process of paragraph 14, wherein the catalyst is platinum nanoparticles, wherein the first support is an aluminum oxide support or silica-aluminum oxide support, and wherein the platinum nanoparticles are dispersed on the surface of the aluminum oxide support or silica-aluminum oxide support.

16. The process of paragraph 14 or 15, further comprising a second support, wherein the first support having the catalyst dispersed thereon is mixed with the second support and the second support is an acidic support.

17. The process of paragraph 16, wherein the second support is a halogenated alumina support, optionally a fluorinated alumina support, and the weight percentage of the halogen, optionally the fluorine, in the second support is in a range from 0.1% to 5%, from 0.5% to 3%, from 0.5% to 2%, from 0.5% to 1.5%, optionally about 0.7% or about 1.3%.

18. The process of any one of paragraphs 14-17, wherein the total weight loading of the metal is in a range from 0.1 wt % to 10 wt %, from 0.1 wt % to 8 wt %, from 0.1 wt % to 5 wt %, from 0.1 wt % to 1 wt %, from 0.5 wt % to 10 wt %, from 0.5 wt % to 8 wt %, from 0.5 wt % to 5 wt %, from 1 wt % to 10 wt %, from 1 wt % to 8 wt %, or from 1 wt % to 5 wt % of the total weight of the catalyst and the first support or the total weight of the catalyst and the first and second support.

19. The process of any one of paragraphs 16-18, wherein the weight percentage of the second support is in a range from 2% to 90%, from 2% to 50%, from 2% to 35%, from 10% to 90%, from 10% to 80%, from 10% to 70%, from 10% to 60%, from 10% to 50%, from 10% to 30%, from 15% to 40%, from 20% to 50%, from 20% to 40%, or from 20% to 35% of the total weight of the catalyst and the first and second support.

20. The process of any one of paragraphs 1-19, wherein during step (ii), the reactor is operated at a temperature of up to 500 °C, up to 450 °C, up to 400 °C, up to 350 °C, up to 320 °C, up to 300 °C, up to 290 °C, between 250 °C and 500 °C, between 250 °C and 450 °C, between 250 °C and 400 °C, between 300 °C and 500 °C, between 320 °C and 500 °C, between 300 °C and 450 °C, between 320 °C and 450 °C, between 300 °C and 400 °C, between 300 °C and 360 °C, between 250 °C and 350 °C, between 250 °C and 350 °C, between 250 °C and 320 °C, or between 250 °C and 300 °C.

21. The process of any one of paragraphs 1-20, wherein during step (ii), the reactor is operated under a H₂ pressure in a range from 1 atm to 8 atm, from 1 atm to 7 atm, from 1 atm to 6 atm, from 1 atm to 5 atm, or from 1 atm to 4 atm.

22. The process of any one of paragraphs 1-21, wherein during step (ii), the reactor is operated for a period of time of up to 24 hours, up to 6 hours, up to 5.5 hours, up to 5 hours, up to 4.5 hours, up to 4 hours, up to 3.5 hours, up to 3 hours, up to 2.5 hours, up to 2 hours, up to 1.5 hours, up to 1 hour, up to 55 minutes, up to 50 minutes, up to 45 minutes, up to 40 minutes, up to 35 minutes, up to 30 minutes, up to 25 minutes, up to 20 minutes, up to 15 minutes, up to 10 minutes, up to 5 minutes, in a range from 5 minutes to 6 hours, from 5 minutes to 5.5 hours, from 5 minutes to 5 hours, from 5 minutes to 4.5 hours, from 5 minutes to 4 hours, from 5 minutes to 3.5 hours, from 5 minutes to 3 hours, from 5 minutes to 2.5 hours, from 5 minutes to 2 hours, from 5 minutes to 1.5 hours, from 1 minute to 1 hour, from 5 minutes to 1 hour, from 10 minutes to 1 hour, from 15 minutes to 1 hour, from 20 minutes to 1 hour, from 30 minutes to 1 hour, from 1 minute to 55 minutes, from 1 minute to 50 minutes, from 1 minute to 45 minutes, from 1 minute to 40 minutes, from 1 minute to 35 minutes, or from 1 minute to 30 minutes.

23. The process of any one of paragraphs 1-22, wherein following steps (i) and (ii), the yield of the alkylbenzene compound in the product is at least 4% or at least 5%.

24. The process of any one of paragraphs 1-23, wherein the hydrocarbon polymer is a high density polyethylene polymer or a low density polyethylene polymer, or a combination thereof.

25. The process of any one of paragraphs 1-24, further comprising a step of (a) feeding hydrogen gas into the reactor during step (i) or after step (i) and prior to step (ii).

26. The process of paragraph 25, further comprising purifying the hydrogen gas prior to step (a).

27. The process of any one of paragraphs 1-26, further comprising a step of (b) feeding hydrogen gas into the reactor during step (ii) to maintain the P(H₂), wherein step (b) occurs one or more times.

28. The process of any one of paragraphs 1-27, further comprising a step of (c) after step (ii), removing hydrogen gas from the reactor and feeding hydrogen gas into the reactor.

29. The process of paragraph 28, wherein following step (c), the reactor is under a second P(H₂) that is different from the P(H₂) in step (ii).

30. The process of paragraph 29, further comprising a step of (d) operating the reactor at a second sufficient temperature, under the second P(H₂), and for a second sufficient period of time to convert unreacted hydrocarbon polymer to a second product comprising a liquid, wherein the liquid comprises an alkylbenzene compound.

31. The process of any one of paragraphs 1-30, further comprising a step of (iii) processing the waste to a suitable form prior to step (i).

32. The process of paragraph 31, wherein the waste is in the form of solid waste, and wherein step (iii) includes shredding, cutting, and/or grinding the waste hydrocarbon polymer to small parts.

33. The process of paragraph 31, wherein the waste is in the form of solid waste, and wherein the process further comprises, prior to step (i), a step of dissolving the solid waste in a solvent, optionally in a hydrocarbon solvent.

34. The process of any one of paragraphs 1-33, further comprising a step of (iv) cooling the reactor to room temperature after step (ii).

35. The process of any one of paragraphs 1-34, further comprising a step of (v) recycling the catalyst after step (ii).

36. The process of any one of paragraphs 25-35, further comprising separating the liquid and wax in the product after step (ii).

37. A composition comprising an alkylbenzene compound, wherein the composition is in the form of a liquid, and wherein the composition is produced by the process of any one of paragraphs 1 to 36.

The disclosed processes and compositions can be further understood through the following enumerated paragraphs.

1. A process for upcycling a waste material, wherein the waste material comprises a hydrocarbon polymer, comprising
(i) feeding the waste material into a reactor,
   wherein the reactor comprises a catalyst therein,
      wherein the catalyst comprises a transition metal; and
(ii) operating the reactor under a sufficient hydrogen gas pressure (P(H₂)) to form a product comprising a liquid comprising one or more alkylbenzene compound(s).

2. The process of paragraph 1, wherein the liquid further comprises one or more other aromatic compounds and optionally one or more alkane and/or one or more non-aromatic compounds.

3. The process of paragraph 2, wherein the mass selectivity of the one or more alkylbenzene compound(s) relative to all aromatic compounds in the liquid is at least 25%, at least 30%, at least 50%, in a range from 25% to 99.99%, from 25% to 99.9%, from 25% to 99%, from 25% to 95%, from 25% to 90%, from 25% to 90%, from 25% to 80%, from 25% to 70%, or from 25% to 60%.

4. The process of any one of paragraphs 1-3, wherein the hydrocarbon polymer is polyethylene, polypropylene, polystyrene, a copolymer of polyethylene and polypropylene, or acrylonitrile butadiene styrene, optionally wherein the hydrocarbon polymer is polyethylene.

5. The process of any one of paragraphs 1-4, wherein the number of carbon atoms in each of the one or more alkylbenzene compound(s) ranges from 7 to 40 carbon atoms or from 10 to 30 carbon atoms.

6. The process of any one of paragraphs 1-5, wherein each of the one or more alkylbenzene compound(s) has a weight average molecular weight (Mw) in a range from 150 g mol⁻¹ to 800 g mol⁻¹, from 200 g mol⁻¹ to 800 g mol⁻¹, from 200 g mol⁻¹ to 600 g mol⁻¹, from 200 g mol⁻¹ to 500 g mol⁻¹, or from 200 g mol⁻¹ to 400 g mol⁻¹.

7. The process of any one of paragraphs 1-6, wherein each of the one or more alkylbenezene compound(s) has a structure of Formula (I):

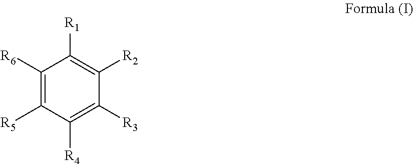

wherein R₁-R₆ are independently hydrogen or an alkyl group, and wherein at least one of R₁-R₆ is an alkyl group, optionally wherein the alkyl group is —CH₂R, and R is a hydrogen, C₁-C₁₉ alkyl group, C₁-C₁₅ alkyl group, a C₁-C₁₂ alkyl group, a C₁-C₁₀ alkyl group, or a C₁-C₅ alkyl group.

8. The process of any one of paragraphs 1-7, wherein the one or more alkylbenzene compound(s) are independently:

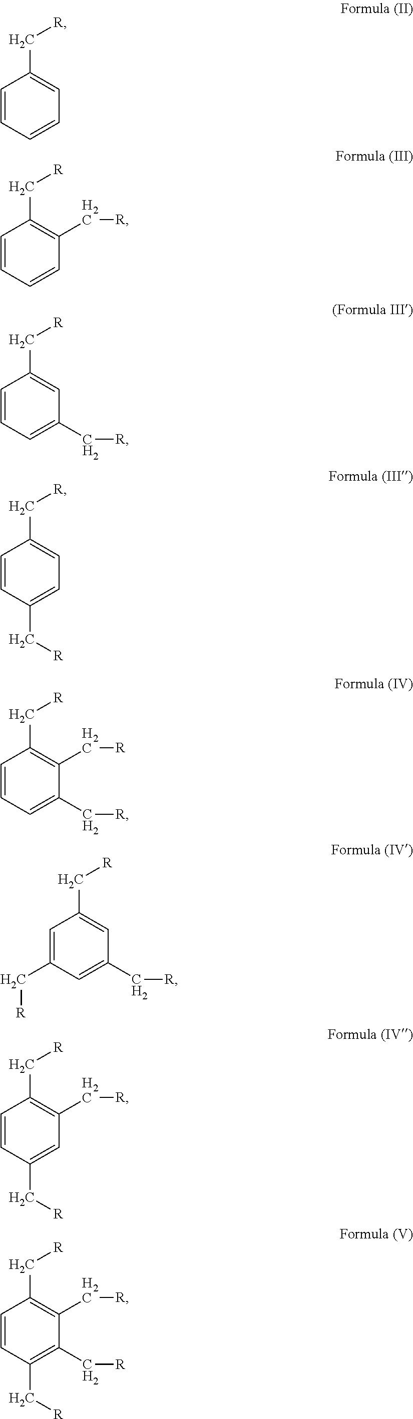

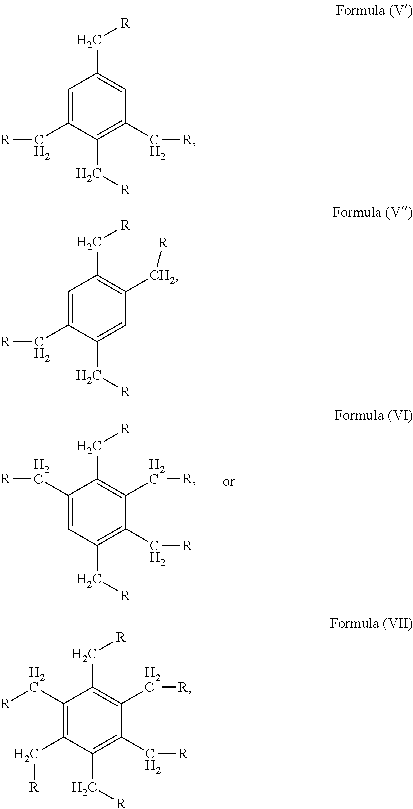

wherein each R is independently hydrogen, a C₁-C₁₅ alkyl group, a C₁-C₁₂ alkyl group, a C₁-C₁₀ alkyl group, or a C₁-C₅ alkyl group.

9. The process of any one of paragraphs 1-8, wherein the one or more alkylbenzene compound(s) are independently:

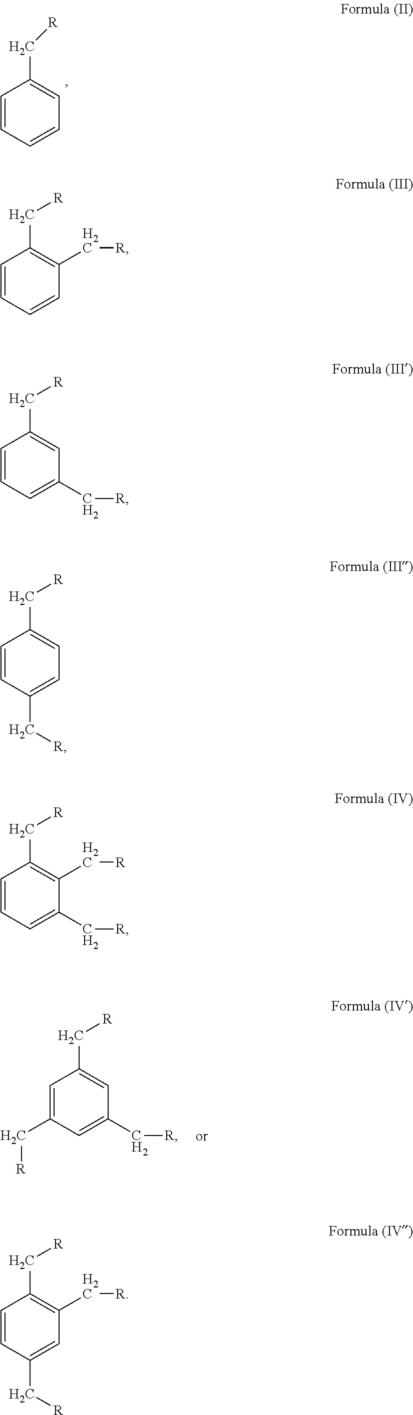

10. The process of any one of paragraphs 1-9, wherein the transition metal is selected from the group consisting of platinum, palladium, ruthenium, iridium, rhenium, rhodium, iron, cobalt, nickel, copper, molybdenum, and tungsten.

11. The process of any one of paragraphs 1-10, wherein the catalyst comprises more than one transition metal, wherein each of the transition metals is independently selected from the group consisting of platinum, palladium, ruthenium, iridium, rhenium, rhodium, iron, cobalt, nickel, copper, molybdenum, and tungsten.

12. The process of paragraph 11, wherein the catalyst further comprises a second metal, wherein the second metal is different from each of the transition metals, and wherein the second metal is selected from the group consisting of rhenium, tin, lead, tungsten, molybdenum, chromium, manganese, and zinc, or a combination thereof

13. The process of any one of paragraphs 1-12, wherein the catalyst is the transition metal, a mixture of two or more metals comprising the transition metal, a metal oxide of the transition metal, or a metal carbide of the transition metal, or a combination thereof.

14. The process of any one of paragraphs 1-13, wherein the catalyst is dispersed on a surface of a first support and wherein the catalyst is in the form of atoms, nanoclusters, or nanoparticles, or a combination thereof.

15. The process of paragraph 14, wherein the catalyst is platinum nanoparticles, wherein the first support is an aluminum oxide support or silica-aluminum oxide support.

16. The process of paragraph 14 or 15, further comprising a second support, wherein the first support is combined with the second support and the second support is an acidic support.

17. The process of paragraph 16, wherein the second support is a halogenated alumina support, optionally a fluorinated alumina support, and the weight percentage of the halogen, optionally the fluorine, in the second support is in a range from 0.1% to 5%, from 0.5% to 3%, from 0.5% to 2%, from 0.5% to 1.5%, optionally about 0.7% or about 1.3%.

18. The process of any one of paragraphs 14-17, wherein the total weight loading of the transition metal is in a range from 0.1 wt % to 10 wt %, from 0.1 wt % to 8 wt %, from 0.1 wt % to 5 wt %, from 0.1 wt % to 1 wt %, from 0.5 wt % to 10 wt %, from 0.5 wt % to 8 wt %, from 0.5 wt % to 5 wt %, from 1 wt % to 10 wt %, from 1 wt % to 8 wt %, or from 1 wt % to 5 wt % of the total weight of the catalyst and the first support or the total weight of the catalyst and the first and second support.

19. The process of any one of paragraphs 16-18, wherein the weight percentage of the second support is in a range from 2% to 90%, from 2% to 50%, from 2% to 35%, from 10% to 90%, from 10% to 80%, from 10% to 70%, from 10% to 60%, from 10% to 50%, from 10% to 30%, from 15% to 40%, from 20% to 50%, from 20% to 40%, or from 20% to 35% of the total weight of the catalyst and the first and second support.

20. The process of any one of paragraphs 1-19, further comprising step (a) feeding hydrogen gas into the reactor during step (i) or after step (i) and prior to step (ii).

21. The process of paragraph 20, further comprising purifying the hydrogen gas prior to step (a).

22. The process of any one of paragraphs 1-21, further comprising step (b) feeding hydrogen gas into the reactor during step (ii) to maintain the P(H₂), wherein step (b) occurs one or more times.

23. The process of any one of paragraphs 1-22, wherein during step (ii), the P(H₂) in the reactor is in a range from 1 atm to 8 atm, from 1 atm to 7 atm, from 1 atm to 6 atm, from 1 atm to 5 atm, or from 1 atm to 4 atm.

24. A process for upcycling a waste material, wherein the waste material comprises a hydrocarbon polymer, comprising
(i) feeding the waste material into a reactor,
   wherein the reactor comprises a catalyst therein,
      wherein the catalyst comprises a transition metal;
(iia) operating the reactor under a first P(H₂) to form fragments of the hydrocarbon polymer, and
after step (iia),
(iib) operating the reactor at a second P(H₂) to form a product comprising a liquid comprising one or more alkylbenzene compound(s),
wherein the second P(H₂) is lower than the first P(H₂).

25. The process of paragraph 24, wherein step (iib) further comprises removing hydrogen gas from the reactor and, optionally, feeding an inert gas into the reactor to provide the second P(H₂), and optionally, an inert gas pressure, in the reactor.

26. The process of paragraph 24 or 25, wherein the first P(H₂) ranges from 5 atm to 20 atm, from 5 atm to 18 atm, from 5 atm to 15 atm, from 5 atm to 12 atm, from 5 atm to 10 atm, such as about 8 atm, and/or the second P(H₂) ranges from 0 to <5 atm, from 0 to 4 atm, from 0 to 3 atm, or from 0 to 2 atm.

27. The process of paragraph 25 or 26, wherein in step (iib), the second P(H₂) is 0 and the reactor is under the inert gas pressure ranging from 0 to <5 atm, from 0 to 4 atm, from 0 to 3 atm, or from 0 to 2 atm.

28. The process of any one of paragraphs 24-27, further comprising step (c) measuring the fragments of the hydrocarbon polymers during step (iia) to determine the weight average molecular weight (Mw) of the fragments, wherein step (c) occurs one or more times.

29. A process for upcycling a waste material, wherein the waste material comprises a hydrocarbon polymer, comprising
(i) feeding the waste material into a reactor,
   wherein the reactor comprises a catalyst therein,
      wherein the catalyst comprises a transition metal;
(iia) operating the reactor under a first P(H₂), to form fragments of the hydrocarbon polymer,
after step (iia),
(iib) operating the reactor at a second P(H₂), to form aromatic compounds, and after step (iib),
(iic) operating the reactor at a third P(H₂), to form a product comprising a liquid comprising one or more alkylbenzene compound(s),
wherein the second P(H₂) is lower than the first P(H₂), and the third P(H₂) is higher than the second P(H₂).

30. The process of paragraph 29, wherein step (iib) further comprises removing hydrogen gas from the reactor and, optionally, feeding an inert gas into the reactor to provide the second P(H₂), and optionally, an inert gas pressure, in the reactor.

31. The process of paragraph 29 or 30, wherein the first P(H₂) ranges from 5 atm to 20 atm, from 5 atm to 18 atm, from 5 atm to 15 atm, from 5 atm to 12 atm, from 5 atm to 10 atm, such as about 8 atm, and/or the second P(H₂) ranges from 0 to <5 atm, from 0 to 4 atm, from 0 to 3 atm, or from 0 to 2 atm.

32. The process of 30 or 31, wherein the second P(H₂) is 0 and the reactor is under an inert gas pressure ranging from 0 to <5 atm, from 0 to 4 atm, from 0 to 3 atm, or from 0 to 2 atm.

33. The process of any one of paragraphs 29-32, wherein step (iic) further comprises feeding hydrogen gas into the reactor to provide the third P(H₂) in the reactor.

34. The process of any one of paragraphs 29-33, wherein third P(H₂) ranges from 5 atm to 15 atm, from 5 atm to 12 atm, or from 5 atm to 10 atm.

35. The process of any one of paragraphs 29-34, further comprising step (c) measuring the fragments of the hydrocarbon polymers during step (iia) to determine the weight average molecular weight (Mw) of the fragments, wherein step (c) occurs one or more times.

36. The process of any one of paragraphs 29-35, further comprising step (d) measuring the aromatic compounds during step (iic) to determine the total yield of the aromatic compounds, wherein step (d) occurs one or more times.

37. The process of any one of paragraphs 24-36, further comprising step (a) feeding hydrogen gas into the reactor during step (i) or after step (i) and prior to step (iia).

38. The process of paragraph 37, further comprising purifying the hydrogen gas prior to step (a).

39. The process of any one of paragraphs 24-38, wherein the liquid further comprises one or more other aromatic compounds and optionally one or more alkane and/or one or more non-aromatic compounds.

40. The process of paragraph 39, wherein the mass selectivity of the one or more alkylbenzene compound(s) relative to all aromatic compounds in the liquid is at least 25%, at least 30%, at least 50%, in a range from 25% to 99.99%, from 25% to 99.9%, from 25% to 99%, from 25% to 95%, from 25% to 90%, from 25% to 90%, from 25% to 80%, from 25% to 70%, or from 25% to 60%,

41. The process of any one of paragraphs 24-40, wherein the hydrocarbon polymer is polyethylene, polypropylene, polystyrene, a copolymer of polyethylene and polypropylene, or acrylonitrile butadiene styrene, optionally wherein the hydrocarbon polymer is polyethylene.

42. The process of any one of paragraphs 24-41, wherein the number of carbon atoms in each of the one or more alkylbenzene compound(s) ranges from 7 to 40 carbon atoms or from 10 to 30 carbon atoms.

43. The process of any one of paragraphs 24-42, wherein each of the one or more alkylbenzene compound(s) has a weight average molecular weight (Mw) in a range from 150 g mol⁻¹ to 800 g mol⁻¹, from 200 g mol⁻¹ to 800 g mol⁻¹, from 200 g mol⁻¹ to 600 g mol⁻¹, from 200 g mol⁻¹ to 500 g mol⁻¹, or from 200 g mol⁻¹ to 400 g mol⁻¹.

44. The process of any one of paragraphs 24-43, wherein each of the one or more alkylbenezene compound(s) has a structure of Formula (I):

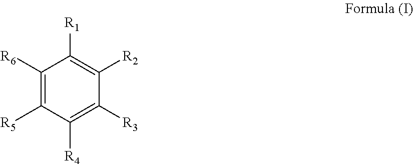

wherein R₁-R₆ are independently hydrogen or an alkyl group, and wherein at least one of R₁-R₆ is an alkyl group, optionally wherein the alkyl group is —CH₂R, and R is a hydrogen, C₁-C₁₉ alkyl group, C₁-C₁₅ alkyl group, a C₁-C₁₂ alkyl group, a C₁-C₁₀ alkyl group, or a C₁-C₅ alkyl group.

45. The process of any one of paragraphs 24-44, wherein the one or more alkylbenzene compound(s) are independently:

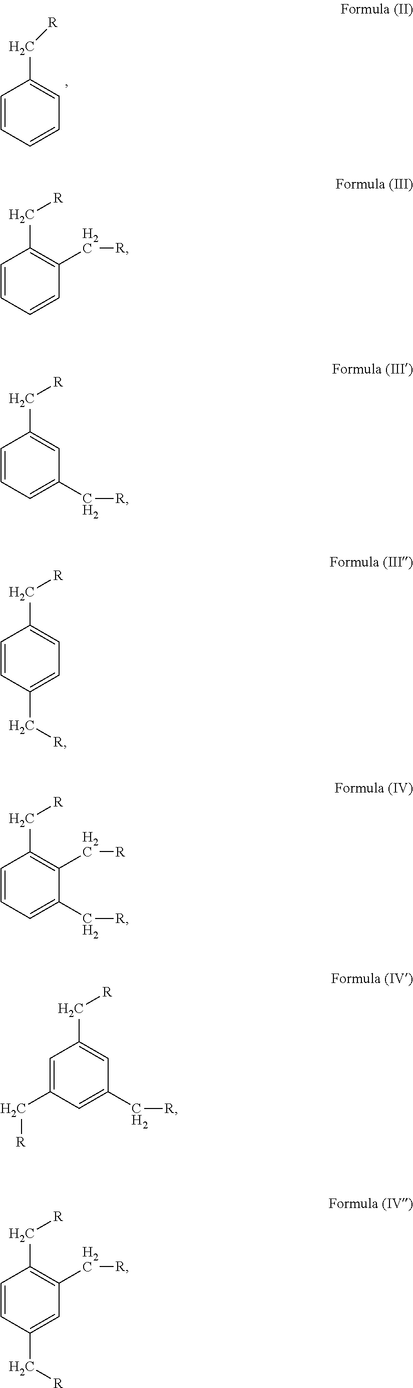

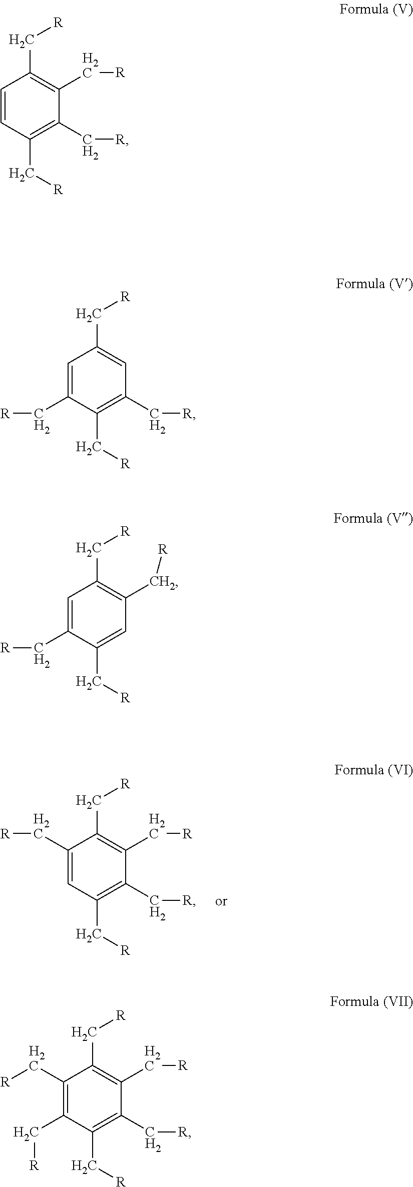

wherein each R is independently hydrogen, a C₁-C₁₅ alkyl group, a C₁-C₁₂ alkyl group, a C₁-C₁₀ alkyl group, or a C₁-C₅ alkyl group.

46. The process of any one of paragraphs 24-45, wherein the one or more alkylbenzene compound(s) are independently:

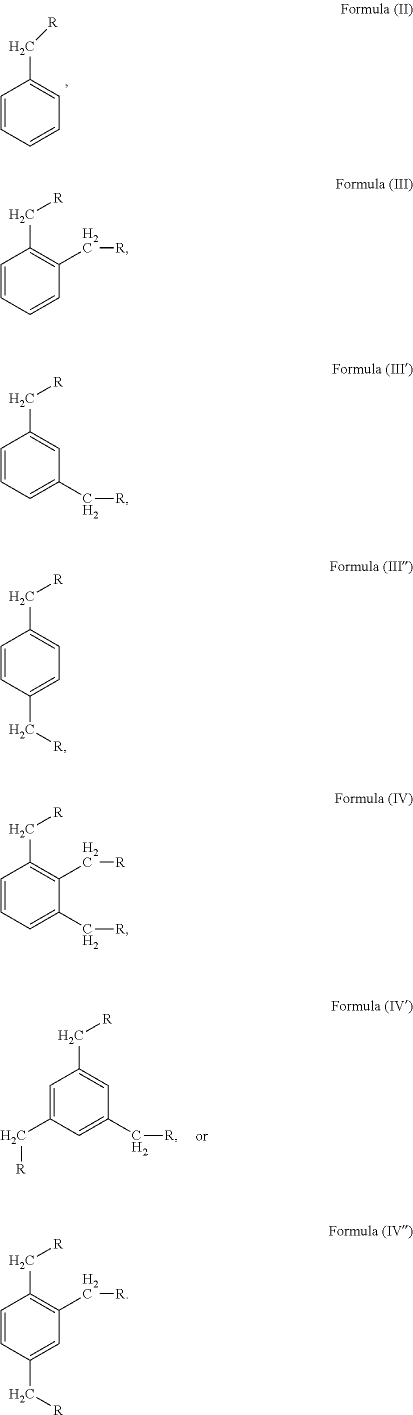

47. The process of any one of paragraphs 24-46, wherein the transition metal is selected from the group consisting of platinum, palladium, ruthenium, iridium, rhenium, rhodium, iron, cobalt, nickel, copper, molybdenum, and tungsten.

48. The process of any one of paragraphs 24-47, wherein the catalyst comprises more than one transition metal, wherein each of the transition metals is independently selected from the group consisting of platinum, palladium, ruthenium, iridium, rhenium, rhodium, iron, cobalt, nickel, copper, molybdenum, and tungsten.

49. The process of paragraph 48, wherein the catalyst further comprises a second metal, wherein the second metal is different from each of the transition metals, and wherein the second metal is selected from the group consisting of rhenium, tin, lead, tungsten, molybdenum, chromium, manganese, and zinc, or a combination thereof

50. The process of any one of paragraphs 24-49, wherein the catalyst is the transition metal, a mixture of two or more metals comprising the transition metal, a metal oxide of the transition metal, or a metal carbide of the transition metal, or a combination thereof.

51. The process of any one of paragraphs 24-50, wherein the catalyst is dispersed on a surface of a first support and wherein the catalyst is in the form of atoms, nanoclusters, or nanoparticles, or a combination thereof.

52. The process of paragraph 51, wherein the catalyst is platinum nanoparticles, wherein the first support is an aluminum oxide support or silica-aluminum oxide support, and wherein the platinum nanoparticles are dispersed on the surface of the aluminum oxide support or silica-aluminum oxide support.

53. The process of paragraph 51 or 52, wherein the first support is combined with a second support and the second support is an acidic support.

54. The process of paragraph 53, wherein the second support is a halogenated alumina support, optionally a fluorinated alumina support, and the weight percentage of the halogen, optionally the fluorine, in the second support is in a range from 0.1% to 5%, from 0.5% to 3%, from 0.5% to 2%, from 0.5% to 1.5%, optionally about 0.7% or about 1.3%.

55. The process of any one of paragraphs 51-54, wherein the total weight loading of the transition metal is in a range from 0.1 wt % to 10 wt %, from 0.1 wt % to 8 wt %, from 0.1 wt % to 5 wt %, from 0.1 wt % to 1 wt %, from 0.5 wt % to 10 wt %, from 0.5 wt % to 8 wt %, from 0.5 wt % to 5 wt %, from 1 wt % to 10 wt %, from 1 wt % to 8 wt %, or from 1 wt % to 5 wt % of the total weight of the catalyst and the first support or the total weight of the catalyst and the first and second support.

56. The process of any one of paragraphs 53-55, wherein the weight percentage of the second support is in a range from 2% to 90%, from 2% to 50%, from 2% to 35%, from 10% to 90%, from 10% to 80%, from 10% to 70%, from 10% to 60%, from 10% to 50%, from 10% to 30%, from 15% to 40%, from 20% to 50%, from 20% to 40%, or from 20% to 35% of the total weight of the catalyst and the first and second support.

57. The process of any one of paragraphs 1-56, wherein the yield of the one or more alkylbenzene compound(s) in the product is at least 4% or at least 5%.

58. The process of any one of paragraphs 1-57, wherein the waste is in the form of a solid, and wherein the process further comprises step (iii) processing the waste prior to step (i), wherein step (iii) includes shredding, cutting, and/or grinding the waste hydrocarbon polymer to small parts, or dissolving the solid waste in a solvent, optionally in a hydrocarbon solvent.

59. The process of any one of paragraphs 25-35, wherein the product further comprises a wax, and wherein the process further comprises step (e) separating the liquid and wax in the product.

60. A composition comprising one or more alkylbenzene compound(s), wherein the composition is in the form of a liquid, and wherein the composition is produced by the process of any one of paragraphs 1 to 59.

61. A composition comprising an alkylbenzene compound having the structure of:

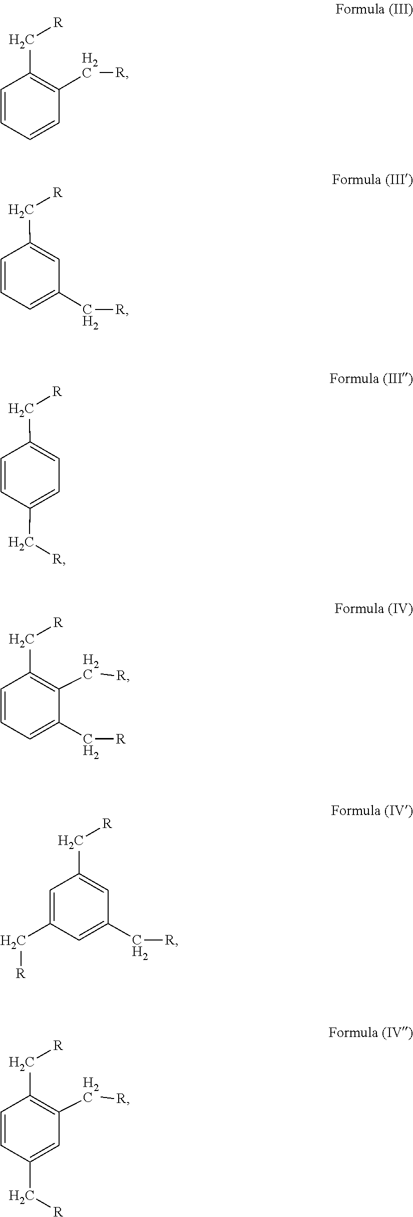

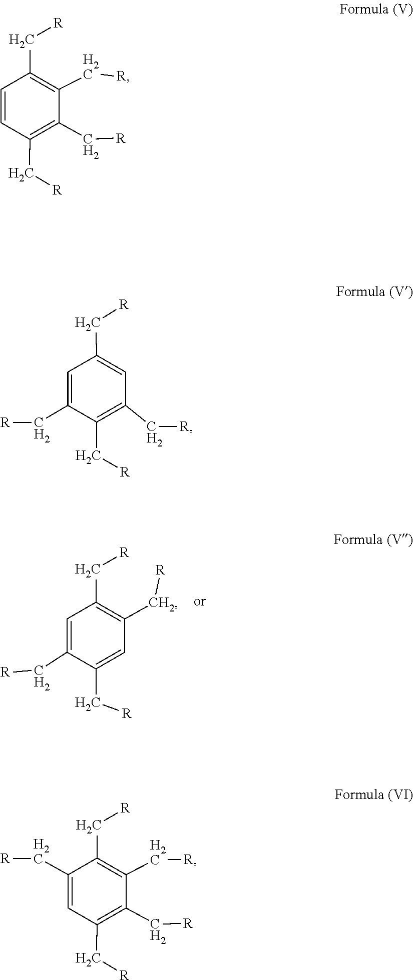

wherein two R groups are independently a linear C₃-C₁₅ alkyl group, a linear C₃-C₁₂ alkyl group, a linear C₃-C₁₀ alkyl group, a linear C₃-C₅ alkyl group, a linear C₄-C₁₅ alkyl group, a linear C₄-C₁₂ alkyl group, a linear C₄-C₁₀ alkyl group, or a linear C₄-C₅ alkyl group, such as a linear C₄ or a linear C₅ alkyl group; and the other R group(s) are hydrogen.

62. The composition of paragraph 61, wherein the alkylbenzene compound has the structure of Formula (III), (III′), (III″), (IV), (IV′), or (IV″).

63. The composition of paragraph 61 or 62, wherein the alkylbenzene compound has the structure of Formula (III), (III′), or (III″).

64. The composition of paragraph 61 or 62, wherein the alkylbenzene compound has the structure of Formula (IV), (IV′), or (IV″).

65. The composition of any one of paragraphs 61-64, wherein the alkylbenzene compound has the structure of

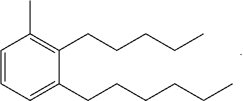 [SMILES: CCCCCCc1cccc(C)c1CCCCC]

The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The present invention will be further understood by reference to the following non-limiting examples. Although the Examples described below are bench scale reactions, the conditions can be scaled up for use in industrial scale reactors to produce alkylbenzenes with the yield and selectivity that are similar to or substantially similar to those produced in the bench scale reactions described below.

### EXAMPLES

### Example 1. Selective Polyethylene Upcycling to Alkylbenzenes Under Moderate Hydrogen Gas Pressure (P(H₂))

### Materials and Methods

### Materials

Trimethyl(cyclopentadienyl)platinum (CpPtMe₃, 99%) was purchased from Strem. Silica-alumina (SiO₂—Al₂O₃, Davicat 3113, 7.6 wt % Al, B.E.T. surface area 573 m² g⁻¹, pore volume 0.76 cm³ g⁻¹) was obtained from Grace-Davison.

A standard Pt solution containing (976±2) mg Pt/kg in 5% w/w HCl, pyridine (anhydrous, 99.8%), polyethylenes of two different molecular weights (Lots SKU 427772 and SKU 427799), dichloromethane (>99.5%), and phenanthrene (analytical standard) were purchased from MilliporeSigma. Nitric acid (68.0-70.0 wt %), hydrochloric acid (36.5-38.0 wt %), and chloroform (HPLC grade, OmniSolv®, CX1054-6) were obtained from EMD Millipore Corp. HPLC-grade water and HPLC-grade triethylamine were purchased from Fisher Chemicals. CD₂°Cl₂ (D, 99.8%) was purchased from Cambridge Isotope Laboratories. All chemicals were used as received.

H₂ (5 vol % in Ar, Airgas Certified Standard) was purified by passing through 13× molecular sieves and BTS catalyst (MilliporeSigma) before use for Pt reduction. Ar (UHP, Airgas) and H₂ (UHP, Airgas) were used in the study of H₂ effects. Propene (99.8%) was obtained from PRAXAir. Carbon monoxide (9.890 vol % in He, Airgas Certified Standard) was used in pulsed CO chemisorption.

### Catalyst Synthesis

Prior to Pt deposition, SiO₂—Al₂O₃ (1.50 g) was first calcined in static air in a temperature-controlled muffle furnace (Thermo Scientific Lindberg/Blue M BF51848 °C) at 500 °C for 4 h. The calcined SiO₂—Al₂O₃ was then dehydrated at ca. 10⁻² Torr and 400 °C for 8 h. Pt/SiO₂—Al₂O₃ was prepared by chemical vapor deposition of CpPtMe₃ (42 mg) onto dehydrated SiO₂—Al₂O₃ (1.5 g), followed by heating to 280 °C at a rate of 2 °C min⁻¹ in flowing H₂ (5.0 vol % in Ar), then holding for 2 h.

### Catalyst Characterization

### Pt Analysis

Pt analysis was performed using Inductively-Coupled Plasma (ICP)—Optical Emission Spectrometry (ICP-OES), on a Thermo iCAP 6300. The calibration curve was constructed using a standard Pt solution, prepared by diluting the commercial Pt standard with homemade aqua regia (5 vol %) in HPLC-grade water. Pt was extracted from catalyst samples following a literature procedure, involving stirring in aqua regia at 75 °C for 30 min. To ensure complete dissolution of Pt, the extraction method was modified by allowing the suspensions to stand at room temperature for an additional 12 h. The suspension was then filtered using a syringe filter (pore size 0.2 μm). The solid residue was washed three times with HPLC-grade H₂O. The filtrate and washings were combined and diluted with aqua regia (5 vol % in water), then analyzed by ICP-OES. The Pt loading was measured to be 1.7 wt %.

Accessible Pt surface areas in the freshly reduced catalyst was measured by CO chemisorption, using a Micrometrics Autochem ii 2920. The catalyst (ca. 40 mg) was placed on a plug of glass wool in a U-shaped quartz reactor. A thermocouple was attached to the outside of the reactor, at the level of the middle of the bed. Complete reduction of the catalyst was ensured by heating in flowing H₂ (5.0 mol % in He) at a rate of 5 °C min⁻¹ to 200 °C, then holding for 1 h before switching to flowing He and cooling to room temperature. The catalyst was subjected to 30°CO pulses (9.9 vol % in He, 124 μL each at 110 °C). The amount of CO not adsorbed in each pulse was measured using a TCD detector. Pt dispersion (D, %) was calculated to be 65% assuming a stoichiometry factor of 1.

TEM images of catalysts were obtained using a ThermoFisher Talos F200X. Pt particle sizes were estimated by analysis of the high angle annular dark field (HAADF) image (Pt/SiO₂—Al₂O₃ (1.7 wt % Pt), data not shown). The average nanoparticle size, obtained by fitting a Gaussian function to the distribution, is (1.0±0.2) nm. The average particle size obtained in this way, (1.0±0.2) nm, was slightly smaller than the Pt particle size estimated by CO chemisorption (1.5 nm, assuming hemispherical Pt nanoparticles). A difference was expected because the TEM method gives a number-average particle size, whereas CO chemisorption gives a surface area-average particle size.

### Acidity Analysis

Brønsted acidity was measured by pyridine adsorption. A sample of dry Pt/SiO₂—Al₂O₃ (ca. 80 mg) was transferred to a Schlenk tube inside an Ar-filled glovebox. The Schlenk tube was sealed, removed from the glovebox, and connected to a vacuum line (base pressure 30 mTorr). The tube was evacuated at 400 °C for 2 h, then cooled to room temperature. Excess dry, air-free pyridine vapor was introduced at room temperature and allowed to equilibrate for 15 min. The tube was then evacuated at ca. 30 mTorr for 30 min while heating at 150 °C to remove physisorbed pyridine. The tube was then transferred into an Ar-filled glovebox, where ca. 10 mg (precisely weighed) was pressed into a self-supporting pellet of diameter 5.0 mm using a hand press (International Crystal Laboratories).

IR spectra were recorded using a Bruker Alpha FTIR spectrometer over the range 4000-400 cm⁻¹ at a resolution of 2 cm⁻¹, accumulating 64 scans. The IR spectrum of Pt/SiO₂—Al₂O₃ (10.0 mg) was recorded after adsorption of excess pyridine at room temperature, followed by desorption of weakly-adsorbed pyridine at 150 °C and ca. 30 mTorr for 30 min. The spectrum of glovebox atmosphere was used as the background. The absorbance was normalized by the precise sample mass. BAS was quantified using the peak areas at 1455 cm⁻¹ (data not shown), using a previously reported IR calibration curve. The area of the peak at 1455 cm⁻¹, corresponding to pyridine interacting with the strong Brønsted acid sites (BAS) of SiO₂—Al₂O₃, was used to quantify the number of strong BAS. The number of Brønsted acid sites on Pt/SiO₂—Al₂O₃ is 42 μmol/g.

### Catalytic Reaction and Product Analysis

### Polyethylene Depolymerization

A Parr reactor (10 mL, models 2550 and 4590; 100 mL, Parr Series 5000 Multiple Reactor System) equipped with a pressure gauge and type J thermocouple was loaded with PE, a freshly-reduced Pt/SiO₂—Al₂O₃ catalyst, and a Pyrex-encapsulated stir bar inside an Ar-filled glovebox. The reactor was sealed and removed from glovebox, which was then connected to the vacuum line (base pressure of 30 mbar) and evacuated. The reactor was then filled with the target pressure of the desired gas, and placed into a heating block. The contents were stirred magnetically at ca. 100 rpm. Timing of the reaction began after ca. 30 min, when the internal reactor temperature reached 275 °C.

After the reaction, the reactor was removed from the heating block and rapidly cooled using a jet of air to room temperature. To characterize volatile products, the reactor and a Schlenk flask capped with a white rubber septum were connected to a glass vacuum line equipped with a pressure gauge and a gas sampling port. The flask and vacuum line were evacuated (10⁻² Torr), then isolated from the pumping system. Gases in the reactor headspace were expanded into the line and Schlenk flask. The pressure reading as recorded before (P₀) and after (P₁) gas expansion.

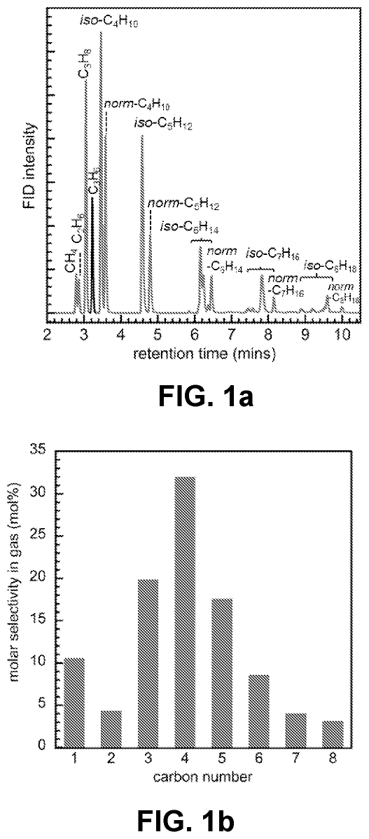

H₂ analysis by GC-TCD was achieved by removing an aliquot of gas (400 L) via the gas sampling port using a gas-tight syringe. Ar present in the autoclave at the start of the reaction was used as an internal standard to quantify H₂ present after reaction by GC-TCD. When Ar was not present, the amount of H₂ was estimated from the pressure (see Section: H₂ Balance). Hydrocarbon gases (mostly C₁ to C₆, m_{C1-C6}) were analyzed by GC-FID. Propene (400 μL, ca. 400 mbar) was injected into the Schlenk tube for use as an internal standard prior to removing a gas aliquot (200 μL) for GC-FID analysis (see, e.g., FIGS. 1a and 1b).

After gas analysis, CD₂°Cl₂ (ca. 3.0 mL) was added to the reactor via the vent hose. The CD₂°Cl₂ and products were stirred at ca. 100 rpm for 5 min, then allowed to stand for 3 min to allow solids to settle. An aliquot of the supernatant (ca. 1 mL) was used for ¹H NMR analysis.

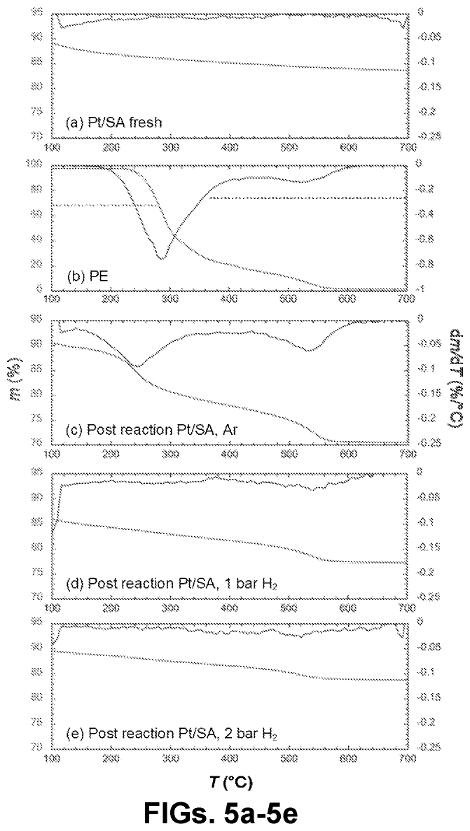

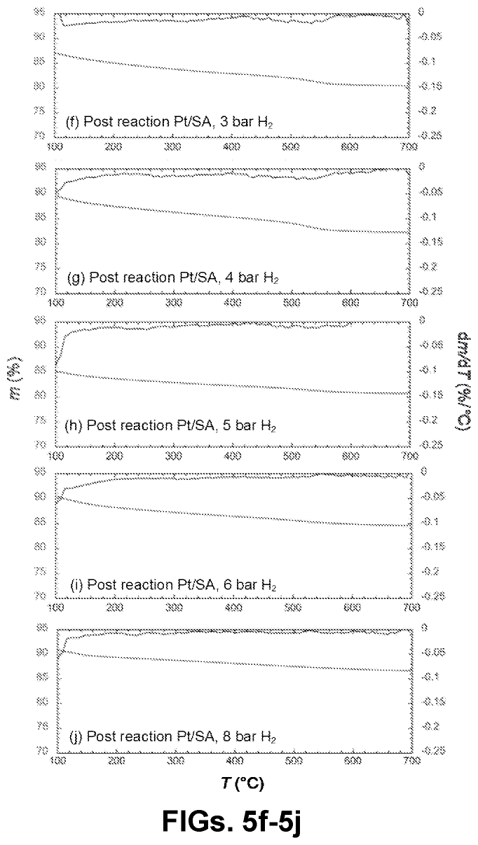

To separate CD₂°Cl₂-soluble hydrocarbons, the suspension was vacuum-filtered through a fine frit (4.0-5.5 μm). The filtrate (including the supernatant for ¹H NMR analysis) was transferred to a volumetric flask and diluted with CH₂°Cl₂ to 25.00 mL. Two aliquots of this solution were used for qualitative and quantitative analysis of liquid hydrocarbons (C₇ to C₃₀) by GC-MS (see FIG. 4 for an example) and GC-FID (see FIG. 5 for an example), respectively. CH₂°Cl₂ and CD₂°Cl₂ were removed under reduced pressure (0.1 Torr, 2 h), resulting in loss of lighter hydrocarbons (C₇-C₁₁). The resulting liquid contains liquid hydrocarbons larger than C₁₂ (C₁₂-C₃₀) and waxes (C_{>30}). The liquid and wax were weighed (mₜₒₜₐₗ) to obtain the mass of wax (eq S1).

m_{wax}=mₜₒₜₐₗ−M_{C12-C30} (S1)

To obtain the molecular weight distribution of the wax, a portion of the remaining liquid/wax (ca. 5 mg) was dissolved in CHCl₃ (1 mL, containing 0.25 vol % triethylamine) for GPC analysis. Typically, a bimodal distribution was observed, with a lower molecular weight peak corresponding to <200 g/mol (see, e.g., FIGS. 4a and 4b). The molecular weight distribution was not accurately reflected in the GPC, and was better characterized by GC-FID, as described above. The higher molecular weight peak contains mostly molecules with carbon numbers C_{>30}, but the error was still significant (the molecular weight of squalane, C₃₀H₆₂, was measured to have 560 g/mol, an error of 30%). The number- and mass-average molecular weights of this wax fraction were calculated using eq S2 and eq S3, respectively. The number-average molecular weight of the combined liquid and wax products was then calculated using eq S4.

$${M}_{n , wax} = \frac{{\sum }_{i} {f}_{i}}{{\sum }_{i} ( \frac{{f}_{i}}{{M}_{i}} )} , {M}_{i} > 420 g / mol \qquad ( S2 )$$

$${M}_{w , wax} = \frac{{\sum }_{i} {f}_{i} {M}_{i}}{{\sum }_{i} {f}_{i}} , {M}_{i} > 420 g / mol \qquad ( S3 )$$

$${M}_{n , liquid / wax} = \frac{{m}_{liquid} + {m}_{wax}}{{n}_{liquid} + \frac{{m}_{wax}}{{M}_{n , wax}}} \qquad ( S4 )$$

where fᵢ is the detector response in the GPC experiment, and Mᵢ is obtained from the GPC retention time based on a calibration curve. Values for both m_{liquid} and n_{liquid} were obtained by GC-FID analysis.

The insoluble material remaining on the frit, including the catalyst and insoluble hydrocarbons, was recovered and weighed. The mass of insoluble hydrocarbons was calculated by subtracting the initial mass of the catalyst. In some cases, the post-extraction solid was characterized by thermogravimetric analysis (FIGS. 5a-5j), giving information on unreacted PE (insoluble in CH₂ °C₂ but fully oxidized in air to CO₂ and H₂O below 350 °C) and carbon residue (insoluble in CH₂°Cl₂ and completely oxidized only above 350 °C in air).

### Depolymerization at Quasi-Constant P(H₂)

In this set of experiments, reactions were conducted in a series of timed stages, each typically 4 h long. The first stage followed the procedure for packing the reactor, conducting the reaction, and analyzing the gas products described in Section: Polyethylene Depolymerization. Between each stage, the reactor headspace was analyzed, then the reactor was evacuated and refilled with Ar or H₂, to keep P(H₂) relatively constant. This procedure was repeated multiple times. In the final stage, the analysis of gas, liquid, wax, and insoluble hydrocarbons was conducted as described in Section: Polyethylene Depolymerization. The total gas products represent the sum of all the gases recovered at the end of each stage.

### H₂ and Hydrocarbon Analysis

H₂ analysis by GC-TCD. H₂ was quantified on a Shimadzu GC-8AIT gas chromatograph equipped with a packed column (ShinCarbon ST 80/100, 2 m×2 mm) and a thermal conductivity detector (TCD). The column, injector and detector temperatures were all 130 °C. The TCD current was 70 mA and the head pressure was 300 kPa (N₂). When Ar was present in the reactor, it was used as the internal standard. The linear responses of the TCD signal to the injected volumes of H₂ and Ar were confirmed using standard H₂/Ar gas mixtures. The response factors were obtained as described previously.

Gas hydrocarbon analysis by GC-FID. Hydrocarbons in the gas fraction (C₁₋₈) were analyzed qualitatively and quantitatively on a Shimadzu GC-2010 gas chromatograph equipped with a capillary column (Supelco Alumina Sulfate plot, 30 m×0.32 mm) and a flame ionization detector (FID). The injector and detector temperatures were both 200 °C. The temperature program was: 90 °C (hold 3 min), ramp 20 °C/min to 180 °C (hold 20 min). The yields of each hydrocarbon were calculated assuming all have the same per-carbon response factor.

Liquid hydrocarbon analysis by GC-FID. The liquid hydrocarbons were quantified on an Agilent 6890N Network gas chromatograph equipped with an Agilent DB-5 capillary column (fused silica, 30 m×0.25 mm×0.25 μm) and an FID detector. The inlet and detector temperatures were 300 and 280 °C, respectively. The temperature ramp program was: 40 °C (hold 3 min), ramp 25 °C/min to 320 °C (hold 10 min). The flow rate was 1.0 mL/min (He) with a 5:1 split ratio. The same per-carbon response factor (f) was assumed for all hydrocarbons. n-Octadecane in CH₂°Cl₂ was used as an external standard. Peak areas were measured by injection of several n-octadecane solutions of different concentrations, giving a calibration curve (data not shown).

GC-MS analysis. An aliquot of the liquid products dissolved in CH₂°Cl₂ (2 mL) was transferred to a vial for solvent removal in a rotary evaporator (30 °C, 350 Torr), then analyzed on a Shimadzu GC-2010 gas chromatograph equipped with an Agilent DB-5 MS capillary column ((5%-phenyl)-methylpolysiloxane, 30 m×0.25 mm×0.25 μm) coupled to a QP2010 Mass Spectrometer. The injector and detector temperatures were 250 °C. The temperature ramp program was: 60 °C (hold 2 min), ramp 15 °C per min to 270 °C, hold 50 min.

NMR spectroscopic analysis. ¹H NMR spectra of liquid products recovered from the reactions of PE were recorded on a Varian Unity Inova AS600 spectrometer, and analyzed by MestReNova (v11.0.1, Mestrelab Research S. L.).

Gel permeation chromatography analysis. Molecular weight distributions of total hydrocarbons and aromatic chromophores were analyzed by GPC with RI and UV detection, respectively. Analyses were conducted on a Waters 2690 HPLC equipped with an Agilent column (PLgel, 5 μm MiniMIX-D, 250×4.6 mm) and a guard column (Mw linear range 200-400,000 g mol⁻¹), a Waters 2410 refractive index (RI) detector and a Waters 2998 photodiode array detector (PDA). Chloroform containing 0.25 vol % triethylamine was used as the mobile phase at room temperature and 0.35 mL min⁻¹. Calibration was achieved using polystyrene standards (Agilent EasiVial kit, linear response for molecular weights in the range 200-400,000 g mol⁻¹).

Thermogravimetric analysis. To assess the nature of hydrocarbon residues post-reaction, solids recovered from the reactor after PE conversion were analyzed by TGA. The measurements were performed in air with a TA Discovery Thermo-Gravimetric Analyzer while heating at 10 °C min⁻¹ from 50 to 700 °C. Differential thermogravimetric analysis shows a distinct peak at ca. 250-350 °C corresponding to the oxidation of unreacted PE, while the peak at ca. 550 °C is assigned to the oxidation of coke (FIGS. 5a-5j).

### Analysis of Aromatic Products

### Analysis of Alkyl Substituents

To understand the type of alkyl substituents present on the aromatic rings, ¹H NMR signals in the region 2.0-3.5 ppm (Hα) due to these alkyl substituents were integrated. The region can be further subdivided into 2.0-2.5 ppm (H_{α-methyl}), 2.5-3.1 ppm (H_{α-methylene}), and 3.1-3.5 ppm (H_{α-methine}). The average number of H_{α} per alkyl substituent, q, was calculated using eq S5.

q=(H_{α,methyl}+H_{α,methylene}+H_{α,methine})/(H_{α,methyl}/3+H_{α,methylene}/2+H_{α,methine}) (S5)

As P(H₂) increases from 0 to 6 bar, the value of q increases slightly from 1.9 to 2.2. This result indicated that for all of these reaction conditions the alkyaromatic products contain, on average, two H_{α} per alkyl substituent (i.e., —CH₂R).

To estimate the average number of alkyl substituents per aromatic ring, the ratio of Hα/Hₐᵣₒₘ (where Hₐᵣₒₘ corresponds to ¹H NMR signals in the range 6.5-9.0 ppm) was evaluated. When P(H₂) was ≤1 bar, alkylphenanthrene was the most abundant aromatic product. Hα/Hₐᵣₒₘ was close to 1.2 when P(H₂) is 0 or 1 bar, indicating that the major products were trialkyl-substituted phenanthrenes. The bifunctional mechanism to make alkylaromatics (Scheme 1a) shows that one —CH₃ and two —CH₂R substituents are likely (Scheme 1b), where the —CH₃ group originates from acid-catalyzed skeletal isomerization of the polymer. The H_{α}/Hₐᵣₒₘ ratio increased further, from 1.3 to 1.7, as P(H₂) increased from 2 to 4 bar, indicating that the major products shifted to trialkyl-substituted naphthalenes. The Hα/Hₐᵣₒₘ. further increased above 2 for P(H₂) values from 5 to 8 bar, corresponding to trialkyl-substituted benzenes. Therefore, trialkyl-substituted benzenes and phenanthrenes were the major aromatic products under moderate, and low P(H₂), respectively.

Scheme 1a. Proposed mechanism for methyldialkylaromatic formation from methyl-branched PE.

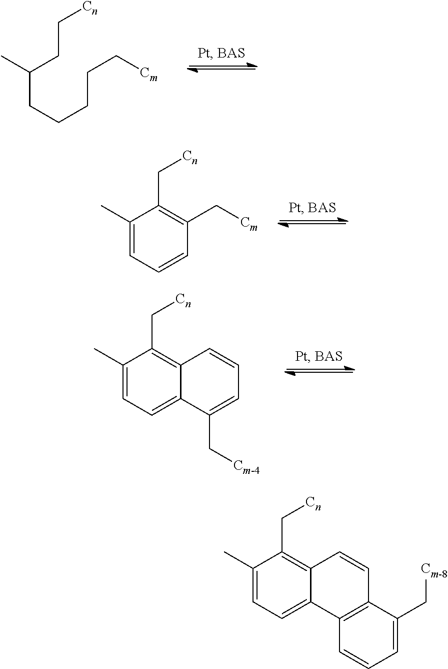

Scheme 1b. Structures of the major alkylaromatic products (trialkyl-substituted phenanthrenes, naphthalenes, and benzenes) formed in the depolymerization of PE (0.200 g, M_{w}=3.5×10³ g mol⁻¹, Ð=1.9) catalyzed by Pt/SiO₂—Al₂O₃ (0.100 g) at 275 °C for 24 h in the presence of 0 to 8 bar external H₂.

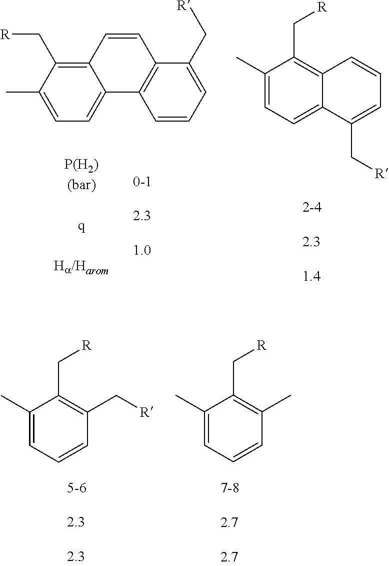

q is the average number of Hₐ per alkyl substituent, according to eq S5 described above.

### Selectivity and Yield of Aromatics

Trialkyl-substituted benzenes, naphthalenes, and phenanthrenes contributed 3, 5, and 7 aromatic protons, respectively. Alkanes were also present in the products. The ratio of aromatic protons to total protons was calculated using eq S6, adapted from the literature.³

$$\frac{{H}_{arom}}{{H}_{total}} = \frac{3 {\sum }_{i} {n}_{ben , i} + 5 {\sum }_{i} {n}_{nap , i} + 7 {\sum }_{i} {n}_{phe , i}}{\begin{matrix}( 2 \overline{{i}_{ben}} - 6 ) {\sum }_{i} {n}_{ben , i} + ( 2 \overline{{i}_{nap}} - 12 ) {\sum }_{i} {n}_{nap , i} + \\ ( 2 \overline{{i}_{phe}} - 18 ) {\sum }_{i} {n}_{phe , i} + ( 2 \overline{{i}_{alk}} + 2 ) {\sum }_{i} {n}_{alk , i}\end{matrix}} \qquad ( S6 )$$

where ben, nap, phe, and alk represent alkylbenzenes, alkylnaphthalenes, alkylphenanthrenes, and alkanes, respectively.

$$[ {\sum }_{i} {n}_{i}$$

and [symbol] represent the total number of moles and the average carbon number for each type of hydrocarbon.

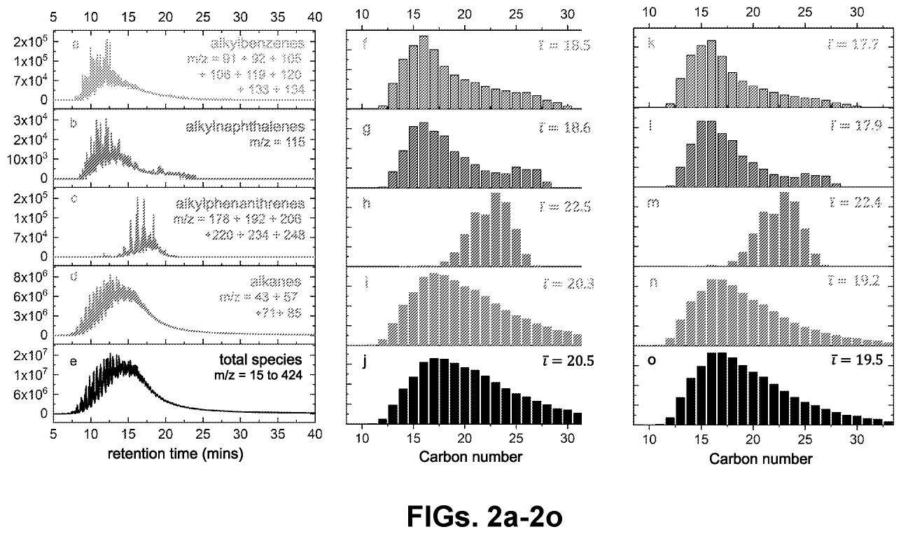

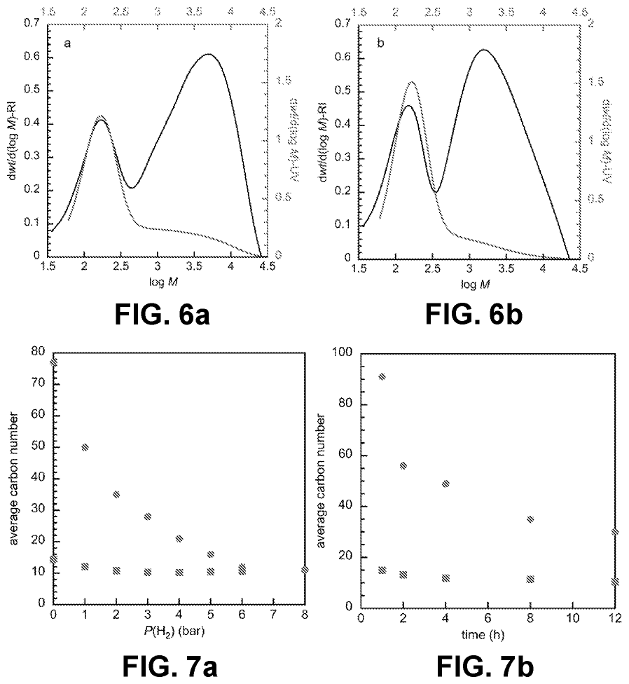

Alkylaromatics were mostly present in the liquid range (C₇-C₃₀, FIGS. 6a, 6b, 7a, and 7b), and their molecular weight distributions were similar to the overall distribution for liquid products (FIGS. 2a-2o). In contrast, alkanes were present in both the liquid and wax fractions. Therefore, the contributions of the liquid and wax fractions to the total protons in eq S6 should be accounted for separately as in eq S7.

$$\frac{{H}_{arom}}{{H}_{total}} = \frac{3 {\sum }_{i , liq} {n}_{ben , i} + 5 {\sum }_{i , liq} {n}_{nap , i} + 7 {\sum }_{i , liq} {n}_{phe , i}}{\begin{matrix}( 2 \overline{{i}_{ben , liq}} - 6 ) {\sum }_{i , liq} {n}_{ben , i} + ( 2 \overline{{i}_{nap , liq}} - 12 ) {\sum }_{i , liq} {n}_{nap , i} + \\ ( 2 \overline{{i}_{phe , liq}} - 18 ) {\sum }_{i , liq} {n}_{phe , i} + ( 2 \overline{{i}_{alk , liq}} + 2 ) {\sum }_{i , liq} {n}_{alk , i} + \text{?}\end{matrix}} \qquad ( S7 )$$

$$\text{?} \text{indicates text missing or illegible when filed}$$

where Σ_{i,wax}n_{alk,i}=Σ_{i,wax}nᵢ, because effectively all hydrocarbons in the wax were alkanes.

The molar selectivity to alkylbenzenes in the liquid fraction (S_{ben.liq}) is defined in eq S8, where Σ_{i,liq}n_{ben,i}+Σ_{i,liq}n_{nap,i}+Σ_{i,liq}n_{phe,i}+Σ_{i,liq}n_{alk,i}+Σ_{i,liq}nᵢ. Molar selectivities for other components are defined similarly. Eq S9 is obtained by combining eqs S7-S8.

$${S}_{ben , liq} = \frac{{\sum }_{i , liq} {n}_{ben , i}}{{\sum }_{i , liq} {n}_{ben , i} + {\sum }_{i , liq} {n}_{nap , i} + {\sum }_{i , liq} {n}_{phe , i} {\sum }_{i , liq} {n}_{alk , i}} \qquad ( S8 )$$

$$\begin{matrix}\frac{{H}_{arom}}{{H}_{total}} = \frac{3 {S}_{ben , liq} + 5 {S}_{nap , liq} + 7 {S}_{phe , liq}}{\begin{matrix}( 2 \overline{{i}_{ben , liq}} - 6 ) {S}_{ben , liq} + ( 2 \overline{{i}_{nap , liq}} - 12 ) {S}_{nap , liq} + \\ ( 2 \overline{{i}_{phe , liq}} - 18 ) {S}_{phe , liq} + ( 2 \overline{{i}_{alk , liq}} + 2 ) {S}_{alk , liq} + ( 2 \overline{i} \text{?}\end{matrix}} & \end{matrix} \qquad ( S9 )$$

$$\text{?} \text{indicates text missing or illegible when filed}$$

To obtain the selectivity for each type of aromatic hydrocarbon, the contributions of methyldialkyl-substituted aromatics to the ¹H NMR signals in the aromatic region were analyzed. The experimental ratios H₁:H₂:H₃ were obtained by integrating the relevant aromatic regions in the ¹H NMR spectrum. These values were related to the molar amounts (nⱼ) for each aromatic structure type to estimate the relative molar selectivities, eq S10.

Sₚₕₑ: Sₙₐₚ: S_{ben}=nₚₕₑ: nₙₐₚ: n_{ben}=1/2H₃: 1/4(H₂−3/2H₃): 1/3H₁−1/12H₂−5/24H₃ (S10)

The average carbon number of alkylbenzenes, alkylnapthalenes, alkylphenanthrenes, and alkanes can be assumed to be similar in the liquid range, eq S11. This was called assumption 1.

$$\overline{{i}_{ben , liq}} = \overline{{i}_{nap , liq}} = \overline{{i}_{phe , liq}} = \overline{{i}_{alk , liq}} \qquad ( S11 )$$

The selectivity for each type of alkylromatic was obtained by solving eqs S9-S11 simultaneously.

Alternatively, since alkylbenzenes, alkylnaphthalenes, and alkylphenanthrenes contain at least 6 carbons, 10 carbons, and 14 carbons, respectively, their average carbon number can be assumed equal to the average carbon number of the alkanes in the ranges C₆₋₃₀, C₁₀₋₃₀, and C₁₄₋₃₀, respectively, eqs S12-14. This was called assumption 2.

$$\overline{{i}_{ben , liq}} = \overline{{i}_{alk , C 6 - 30}} \qquad ( S12 )$$

$$\overline{{i}_{naphthalene , liq}} = \overline{{i}_{alk , C 10 - 30}} \qquad ( S13 )$$

$$\overline{{i}_{phenanthrene , liq}} = \overline{{i}_{alk , C 14 - 30}} \qquad ( S14 )$$

The small difference in the results using either of these assumptions was illustrated. For simplicity, reported selectivities were calculated using assumption 1 unless otherwise stated. The yield of each product type was calculated using eq S8 after obtaining the selectivity as mentioned above, and the total liquid fraction products,

$$⌈ {\sum }_{i , liq} {n}_{i} ,$$

was measured by GC-FID.

### Analysis of Carbon-Carbon Bond Scission

The number of C—C bond scission events (n_{C-C scission}) was calculated by quantifying the change in the number of hydrocarbons at the start of the reaction (N(0)) and at time t (N(t)), eqs S15-17.

n_{C-C scission}=N(t)−N(0) (S15)

N(t)=n_{gas}+n_{liquid}+n_{wax}+n_{solid} (S16)

N(0)=n_{PE} (S17)

where the final number of solid hydrocarbon chains (n_{solid}) and the initial number of PE chains (n_{PE}) were estimated as m_{solid}/Mn_{PE} and m_{PE}/Mn_{PE}, respectively. Here, m_{solid} and m_{PE} refer to the eventual mass of solid residue and the initial mass of PE, respectively, while M_{nPE} represents the initial number-averaged molecular weight of PE.

To correct for incomplete mass recovery, the missing mass (200 mg−mₜₒₜₐₗ) was assumed to have the same distribution as the recovered mass (mₜₒₜₐₗ=m_{gas}+m_{liquid}+m_{wax}+m_{solid}). Consequently, the mass-corrected number of C—C bond scission events was calculated as shown in eq S18.

n_{C-C scission,corr}=n_{C-C scission}+(200−mₜₒₜₐₗ)/mₜₒₜₐₗ·N(t) (S18)

Alternatively, the missing mass may be attributed to incomplete collection of gas (C₁-C₆), light liquid (C₇-C₁₁), heavy liquid (C₁₂-C₃₀), wax (C_{>30}), or solid (C_{insoluble}). Because C₆ hydrocarbons can be lost during the depressurization of the reactor, and also the signals for the C₆ hydrocarbons overlap with the solvent peak in GC-FID analysis, the missing mass was thus assumed to come from the underestimation of C₆ (molecular weight denoted M_{C6}). This assumption gave the mass-corrected number of C—C bond scission events shown in eq S19.

n_{C-C scission.c6corr}=n_{C-C scission}+(200−mₜₒₜₐₗ)/M_{C6} (S19)

The uncorrected number of C—C bond scission events and the corrected value assuming the missing mass being C₆ represent lower and upper limits for the number of C—C bond scission events, respectively.

### H₂ Balance

In the catalytic conversion of PE, aromatic formation generated H₂, in an amount (n_{H2,arom}) which was the sum of the degrees of unsaturation of benzenes, naphthalenes, and phenanthrenes, eq S20. At the same time, C—C bond scission consumes H₂, in an amount (n_{H2,C-C}) represented by eq S21, where n_{C-C scission} was calculated by eq S15.

n_{H2,arom}=4n_{benz}+7nₙₐₚₕ+10nₚₕₑₙ (S20)

n_{H2,C-C}=n_{C-C scission} (S21)

When the Parr reactor (volume Vᵣ=10 mL) was filled initially with Ar, the amount of H₂ present after reaction (n_{H2,final}) was measured by GC-TCD using Ar as the internal standard. The peak areas for H₂ (A_{H2}) and Ar (A_{Ar}) were related as shown in eq S22. Response factors for H₂ and Ar reported in the literature were used here.

$$\frac{{n}_{H 2 , final}}{{n}_{Ar}} = \frac{{V}_{H 2}}{{V}_{Ar}} = \frac{{A}_{H 2} / {f}_{H 2}}{{A}_{Ar} / {f}_{Ar}} \qquad ( S22 )$$

When H₂, not Ar, was not present initially, the amount of H₂ after reaction was estimated by measuring the gas pressure in the reactor. The reactor was connected to a vacuum connector and a Schlenk flask (total volume Vᵥₛ), initially at pressure reading P₀=0. The gases in the reactor were allowed to expand into the vacuum connector and Schlenk flask, resulting in a pressure reading P₁. The total moles of gas were calculated using the ideal gas law, eq S23.

$${n}_{total gas} = \frac{{P}_{1} ( {V}_{vs} + {V}_{r} ) - {P}_{0} {V}_{vs}}{RT} \qquad ( S23 )$$

The moles of H₂ were determined by subtracting the moles of gaseous hydrocarbons (n_{gas hydrocarbon}) measured by GC-FID, eq S24.

n_{H2,final}=n_{total gas}−n_{gas hydrocarbon} (S24)

Amount of H₂ predicted to remain at the end of the reaction (n_{H2,pred}) based on the initial amount of H₂ (n_{H2,initial}) before reaction, the amount of generated H₂ by aromatization, and the amount of consumed H₂ by C—C bond scissions were described in eq S25. In most experiments, the value of predicted H₂ corresponded reasonably well to the measured value, demonstrating that the hydrogen balance was well described by these two major processes.

n_{H2,pred}=n_{H2,initial}+n_{H2,arom}−n_{H2C-C} (S25)

### Results and Discussion

### PE Depolymerization in the Absence of External H₂

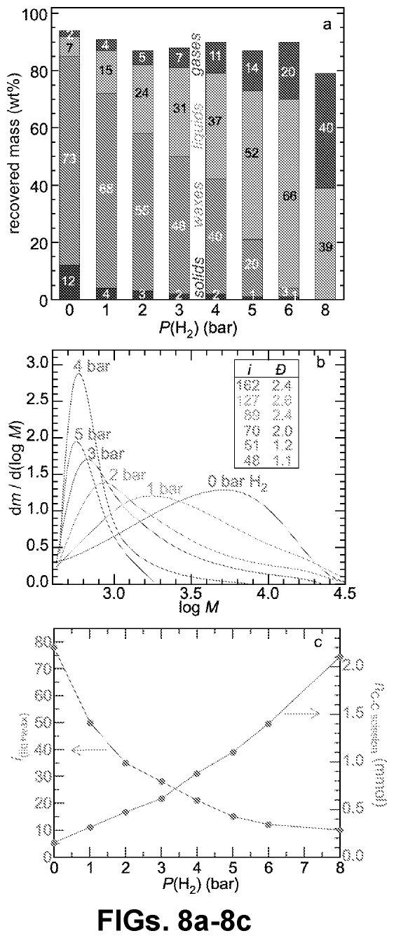

PE depolymerization in the absence of external H₂. Depolymerization was studied initially using a model PE (0.200 g, M_{w}=3.5×10³ g mol⁻¹, Ð=1.9) and a highly acidic bifunctional catalyst, Pt/SiO₂—Al₂O₃ (0.100 g, 7.6 wt % Al, 1.7 wt % Pt at 65% dispersion) under Ar (i.e., in the absence of external H₂) at 275 °C. The yield of light hydrocarbon gases (C₁₋₆) was very low (2 wt %). Prior to reaction, the PE was insoluble in CH₂ °C₁₂, but after 24 h, most hydrocarbons (80 wt %, relative to the initial charge of PE) dissolved in room temperature CH₂°Cl₂(FIGS. 8a-8c and Table 1). This change in solubility was consistent with a significant reduction in molecular weight.

TABLE 1
Effect of P(H₂) on the distribution of hydrocarbon products generated in catalytic PE depolymerization ^{a}

| | Mass fractions of hydrocarbon product types | | | | Mass | | |
|---|---|---|---|---|---|---|---|
| P(H₂) ^{b} | (wt %) | | | | recovery ^{c} | | n_{c-c scission,corr} ^{e} |
| (bar) | Gas ^{f} | Liquid ^{g} | Wax ^{h} | Solid ⁱ | (wt %) | [symbol] | (mmol) |
| 0 | 2 | 7 (12) | 73 (162) | 12 (6) | 94 | 78 | 0.15 |
| 1.0 | 4 | 15 (13) | 68 (127) | 4 (1) | 91 | 50 | 0.31 |
| 2.0 | 5 | 24 (15) | 55 (90) | 3 (1) | 88 | 35 | 0.47 |
| 3.0 | 7 | 31 (14) | 48 (70) | 2 (1) | 88 | 28 | 0.61 |
| 4.0 | 11 | 37 (12) | 40 (51) | 2 (1) | 90 | 21 | 0.88 |
| 5.0 | 14 | 52 (12) | 20 (48) | 1 (0) | 87 | 15 | 1.1 |
| 6.0 | 20 | 66 (11) | 3 (31) | 1 (0) | 90 | 13 | 1.4 |
| 8.0 | 40 | 39 (10) | 0 | 0 | 79 | 10 | 2.1 |
| 0 ^{j} | 3 | 7 (14) | 50 (126) | 32 (13) | 92 | 64 | 0.19 |
| 4.0 ^{j} | 6 | 47 (13) | 27 (59) | 3 (1) | 83 | 18 | 0.88 |

^{a} Reaction conditions: PE (0.200 g, M_{w} = 3.5 × 10³ g mol⁻¹, Ð = 1.9), Pt/SiO₂-Al₂O₃ (0.100 g, 1.7 wt % Pt), magnetically stirred 10 mL Parr reactor, 275 °C, 24 h, except as noted below. In general, reproducibility is indicated by the number of significant figures.
^{b} Measured at room temperature at the start of the experiment.
^{c} Sum of masses for recovered gas, liquid, wax and solid hydrocarbons.
^{d} Average carbon number for combined liquid and wax fractions, measured by GC-FID and GPC, respectively, and estimated as Mₙ/14.
^{e} Number of C—C bond scission events in 24 h, calculated as N(24)-N(0), where N(24) and N(t) represent the total numbers of hydrocarbon chains present initially and after 24 h, respectively. The missing mass (corresponding to a mass balance below 100 %) was assumed to have the same molecular weight distribution as the recovered mass (see the Section: Analysis of Carbon-Carbon Bond Scission).
^{f} C₁-C₆, as measured by GC-FID.
^{g} C₇-C₃₀, as measured by GC-FID. The value in parentheses is the number-averaged carbon number.
^{h} C_{>30}, measured by GPC-RI and calibrated using PS standards. The value in parentheses is the number-averaged carbon number, estimated as Mₙ/14, where Mₙ was calculated using eq S2.
ⁱ Measured by TGA, except where noted. The value in parentheses is the relative amount of unreacted PE, considered to be the carbon that is fully oxidized in air below 350 °C.
^{j} A higher molecular weight PE (0.200 g, M_{w} = 2.6 × 10⁴ g mol-1, Ð = 3.9) was used in this experiment.

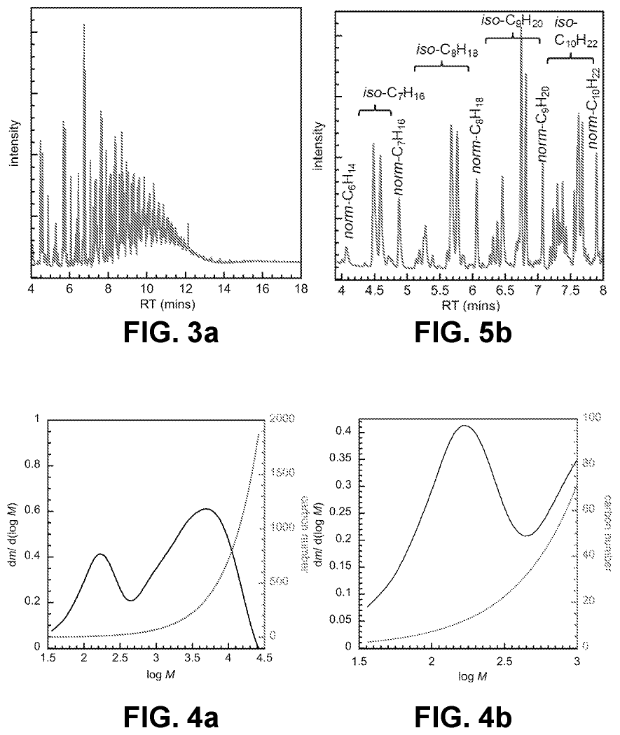

The brown color of the liquid product indicated aromatic formation, specifically, fused-ring aromatics that absorb in the visible. Iso-alkanes were the major paraffinic products in both the gas and liquid phases (FIGS. 1a, 1b, 3a, and 3b), consistent with C—C bond scission by an acid-catalyzed carbenium ion mechanism. Based on the aromatic yield (Table 2), the amount of H₂ originating from aromatization should be 0.5 mmol. Therefore, depolymerization proceeds via tandem hydrocracking/aromatization, with 30% of the H₂ generated by aromatization being consumed by C—C bond scission reactions.

TABLE 2
Effect of P(H₂) on the liquid/wax products of PE depolymerization ^{a}

| | | | | | | | m_{benz}/ | mₐᵣₒₘ/ | m_{benz}/ |
|---|---|---|---|---|---|---|---|---|---|
| P(H₂) ^{b} | P(H₂) ^{c} | nₐₗₖ ^{d} | n_{benz} ^{d} | nₙₐₚₕ ^{d} | nₚₕₑₙ ^{d} | nₐᵣₒₘ ^{e} | m_{arom f} | mₜₒₜₐₗ ^{g} | m_{PE} ^{h} |
| (bar) | (bar) | (μmol) | (μmol) | (μmol) | (μmol) | (μmol) | (%) | (%) | (wt %) |
| 0 | 1.2 | 30 | 5 | 8 | 41 | 54 | 9 | 65 | <1 |
| 1.0 | 1.4 | 118 | 8 | 4 | 32 | 44 | 19 | 27 | 1 |
| 2.0 | 1.8 | 203 | 10 | 6 | 24 | 40 | 27 | 17 | 1 |
| 3.0 | 2.3 | 275 | 18 | 11 | 11 | 39 | 44 | 12 | 2 |
| 4.0 | 2.6 | 383 | 22 | 10 | 7 | 38 | 56 | 9 | 2 |
| 5.0 | 3.0 | 578 | 20 | 4 | 2 | 26 | 70 | 5 | 2 |
| 6.0 | 3.1 | 722 | 14 | 1 | 1 | 16 | 90 | 2 | 1 |
| 8.0 | 3.4 | 549 | <1 | 0 | 0 | <1 | 100 | <1 | <1 |
| 0 ⁱ | 1.2 | 22 | 10 | 8 | 32 | 50 | 20 | 69 | 1 |
| 4.0 ⁱ | 3.0 | 485 | 30 | 5 | 6 | 41 | 73 | 8 | 3 |

^{a} Reaction conditions: PE (0.200 g, M_{w} = 3.5 × 10³ g mol⁻¹, Ð = 1.9), Pt/SiO₂-A1₂O₃ (0.100 g, 1.7 wt % Pt), 275 °C, 24 h, 10 mL, in a stirred Parr reactor. In general, reproducibility is indicated by the number of significant figures.
^{b} H₂ pressure measured before the reaction at room temperature.
^{c} H₂ pressure measured after the reaction at room temperature (see Section: Analysis of H₂ Balance.
^{d} Yields of alkanes (nₐₗₖ), alkylbenzenes (n_{benz}), alkylnaphthalenes (nₙₐₚₕ), and alkylphenanthrenes (nₚₕₑₙ) in liquids, estimated by combining the integrated ¹H NMR intensity and the GC-FID product distribution (see Section: Selectivity and Yield of Aromatics).
^{e} Total molar yield of alkylaromatics, calculated as nₐᵣₒₘ = n_{benz} + nₙₐₚₕ + nₚₕₑₙ.
^{f} Mass selectivity of alkylbenzene relative to total aromatics.
^{g} Mass selectivity of alkylaromatics relative to total hydrocarbons (mₜₒₜₐₗ) in liquids, where mₜₒₜₐₗ = mₐₗₖ + m_{benz} + mₙₐₚₕ + mₚₕₑₙ.
^{h} Mass yield of alkylbenzene relative to the initial mass of PE.
ⁱ A higher molecular weight PE (0.200 g, M_{w} = 2.6 × 10⁴ g mol⁻¹, Ð = 3.9) was used in this experiment.

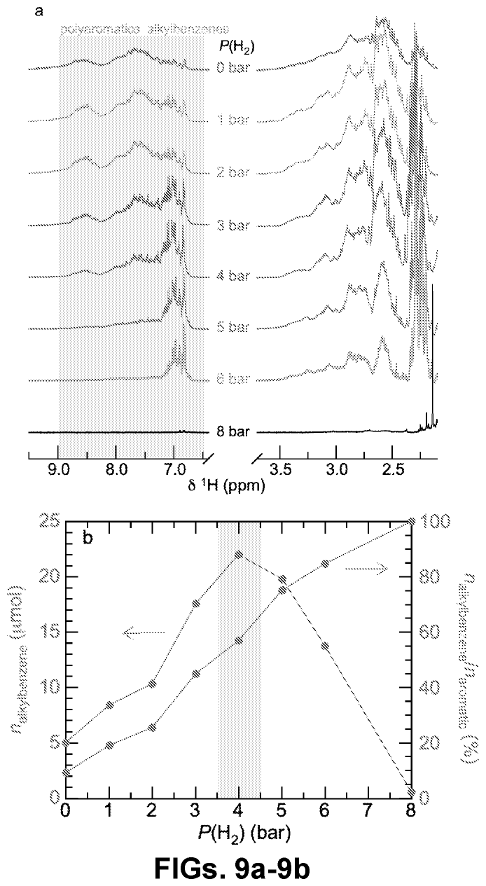

In the ¹H NMR spectrum of the combined liquid and wax products (FIG. 9a), signals from 6.5 to 7.4 ppm can be assigned to alkylbenzenes (H_{benz}), while signals from 7.4 to 9.0 ppm can be assigned to polyaromatics (H_{poly}), where Hₐᵣₒₘ=H_{benz}+H_{poly}. The ¹H NMR-derived ratio H_{benz}/Hₐᵣₒₘ was 0.31. The molar selectivity for alkylbenzenes relative to all alkylaromatics (include alkylnaphthalenes and alkylphenanthrenes) was therefore moderate in the absence of external H₂. The spectrum resembled that for H₂-free PE depolymerization catalyzed by metal-free SiO₂—Al₂O₃ at 280 °C for 24 h, consistent with high catalyst acidity being responsible for polyaromatic formation.

### Effect of External H₂ on PE Chain Scission

When PE depolymerization was conducted under 8 bar H₂ (corresponding to 3.2 mmol H₂), the CH₂°Cl₂-soluble products were colorless and formed in a smaller amount (39 wt %). Instead, the reaction generated much more light hydrocarbon gases (40 wt %), amounting to nearly 14× more C—C bond scission events in the same reaction time. Over half of the external H₂ was consumed, leaving 1.4 mmol H₂ present at the end of the reaction. This result shows that PE hydrocracking to saturated alkanes becomes the dominant reaction when enough H₂ is present.

To probe the influence of H₂ pressure on C—C bond scission and alkane aromatization, reactions were also conducted with external P(H₂) values from 1 to 7 bar. As P(H₂) increased, the total number of C—C bond scission events increased, the average carbon number in the hydrocarbon products decreased, and the major product type shifted gradually from waxes to liquids, then to gases (FIG. 8a). The overall average carbon number for the combined liquid and wax products [symbol] also decreased gradually (FIGS. 8b and 8c), showing random chain scission instead of end-of-chain scission. Regardless of whether the depolymerization was conducted with or without H₂, little CH₄ was formed (<3 wt % of total gas).

### Effect of External H₂ on PE Aromatization

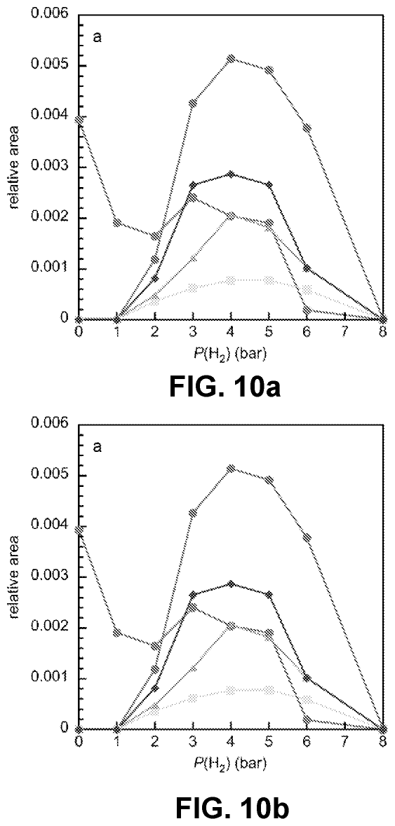

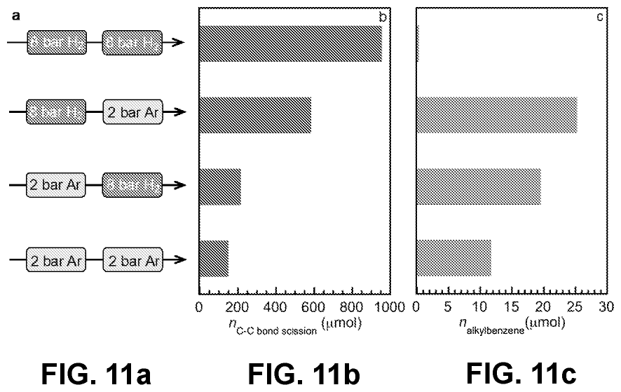

Moderate external P(H₂) significantly improved the selectivity for alkylbenzenes relative to polyaromatics, according to FIG. 9a. Thus, the alkylbenzene selectivity increased from 10 to 100 mol % as P(H₂) increased from 0 to 8 bar (FIG. 11b). GC-MS analysis also confirmed that the characteristic ions of the aromatic products shifted from polyaromatics to monoaromatics as P(H₂) increased (FIG. 10b). Further, quantitative analysis by GC-FID and ¹H NMR (see Section: Selectivity and Yield of Aromatics) showed that the alkylbenzene yield increased with P(H₂) from 0 to ca. 4 bar, then decreased as P(H₂) increases further (FIG. 9b and Table 2). At ca. 4 bar, the maximum alkylbenzene yield was ca. 4× the yield obtained without external H₂. A maximum in the yield of alkylnaphthalenes was observed at ca. 3 bar, but the yield of alkylphenanthrenes decreased monotonically with increasing P(H₂) (Table 2).

GC-MS analysis further revealed that the yields of alkyltetralins and alkyldecalins (i.e., alkyltetrahydronaphthalenes and alkyldecahydronaphthalenes, respectively) increased initially with P(H₂), then decreased (FIG. 10a). To explain this observation, the Gibbs' free energies of three model reactions (the conversion of n-dodecane to 1,2-dipropylbenzene, 2,3-dimethyltetralin, and 2,3-dimethylnaphthalene, respectively) were calculated. As P(H₂) increased, the conversion of n-dodecane to 1,2-dipropylbenzene was more thermodynamically favored relative to the formation of 2,3-dimethyltetralin and 2,3-dimethylnaphthalene (data not shown). As P(H₂) increased further, the formation of 1,2-dipropylbenzene also became unfavorable. The thermodynamic yields of both 1,2-dipropylbenzene and 2,3-dimethyltetralin were predicted to increase with P(H₂) then decrease (data not shown), a trend that is consistent with the experimental observations.

The aromatic substituents in the products of PE depolymerization were a mixture of methyl and longer alkyl chains, where the latter was mostly unbranched at Ca, as indicated by ¹H NMR signals at 2.0-2.5 ppm (C_{α}H₃) and 2.5-3.1 ppm (C_{α}H₂) (FIG. 9a). As P(H₂) increased from 0 to 6 bar, the fraction of methyl substituents increased from 20 to 46%, while the fraction of longer alkyl substituents decreased from 70 to 37% (see Section: Analysis of Alkyl Substituents). This shift suggested more PE isomerization to create methyl branches, as well as more alkyl chain cleavage. On average, the aromatic products were dialkylmethyl-substituted (Schemes 1a and 1b). This result is similar to the products obtained from PE depolymerization catalyzed by acidic Pt/F—Al₂O₃.

The molecular weight distribution of the aromatic products was analyzed by GPC with UV detection. When the reaction was conducted without external H₂ after 24 h at 275 °C, the distribution was bimodal (FIG. 6a). Most aromatics were present in the liquid phase (C₇-C₃₀, 71 wt % relative to total mass of liquid and wax) with a smaller fraction in the wax phase (C_{>30}, 29 wt %, relative to total mass of liquid and wax). With external P(H₂)≥2 bar, the UV absorption of the wax component decreased further (FIG. 6b), such that aromatic products were present only in the liquid phase. The overall average carbon number of the alkylaromatics was ca. C₁₅ without the addition of external H₂, decreasing to C₁₀ as P(H₂) increased to 6 bar (FIG. 7a). In contrast, the average carbon number of the full product mixture decreased much more, from C₇₇ to C₁₂, as external P(H₂) increased from 0 to 6 bar. If assuming the formation of aromatics on the alkane chain was random, shorter chain length of alkylaromatics compared to alkanes could be due to much faster C—C bond scission for alkylaromatics relative to alkanes with the same carbon number. GC-MS further revealed that aromatics were distributed similarly to alkanes in the liquid products obtained under 4 bar H₂ (FIGS. 2a-2o). Therefore, the average carbon number of the aromatic products was the same as the average for all liquid hydrocarbons.

### Behavior of a Higher Molecular Weight PE

Depolymerization of a higher molecular weight PE (M_{w}=2.6×10⁴ g mol−³, Ð=3.9), closer to commercial grade PE, was tested with Pt/SiO₂—Al₂O₃ at 275 °C for 24 h. Similar to the behavior of low molecular weight PE, moderate P(H₂) enhanced C—C bond scission, as well as improving the yield of and selectivity to alkylbenzenes. The number of C—C bond scission events occurring under 4 bar H₂ increased by 0.69 mmol, compared to the same reaction conducted under 2 bar Ar. A similar increase (0.73 mmol) was observed in the reaction of the low molecular weight PE (Table 1). The increases in alkylbenzene yields were 20 and 17 μmol for the higher and lower molecular weight PEs, respectively. Similarly, the increases in alkylbenzene selectivity relative to total alkylaromatic were 53 and 47 mol % for the higher and low molecular weight PEs, respectively (Table 2).

### Varying Reactor P(H₂) to Increase Alkylbenzene Production

The results above showed that moderate H₂ pressure (e.g., 8 bar) was effective in rapidly decreasing the PE molecular weight, but completely suppressed the aromatic yield, while the absence of external H₂ resulted in a slower depolymerization which maximized the aromatic yield but at the expense of alkylbenzene selectivity. Some of the tradeoffs in choosing a single reactor atmosphere may be overcome by varying the headspace gas over the course of a single experiment. For example, the average hydrocarbon molecular weight (and viscosity) would decrease rapidly if the reaction were initiated under moderate P(H₂), then the aromatic yield could be maximized by removing the H₂ from the reactor atmosphere. Alternatively, a high rate of aromatization could be achieved by starting the reaction under Ar, then introducing moderate P(H₂) to convert polyaromatics and higher molecular weight alkylbenzenes to the desired surfactant-range alkylbenzenes.

The potential for dynamic control of the reactor atmosphere was explored in a series of preliminary experiments involving depolymerization of a model PE catalyzed by Pt/SiO₂—Al₂O₃ at 275 °C for two consecutive 4 h periods (data not shown). After the first 4 h period, conducted starting under either 2 bar Ar or 8 bar H₂, the reactor was cooled to room temperature and gases were removed from the reactor headspace for analysis by GC-FID. Next, the same reactor was charged with either 2 bar Ar or 8 bar H₂ for a second 4 h period under the same reaction conditions. When both 4-h reaction stages were conducted under Ar, little C—C bond scission occurred, the average carbon number in the liquid/wax hydrocarbon products was high ( [symbol] =61), and the yield of alkylbenzenes, 12 μmol, was low, representing only 29 mol % of the aromatics produced (Table 3). When both 4-h reaction stages were conducted under 8 bar H₂, a significant increase in C—C bond scission was observed. Consequently, the average carbon number in the liquid/wax hydrocarbon products was much lower ( [symbol] =21), but the yield of alkylbenzenes was negligible.

TABLE 3
Effect of changing the reactor headspace gas during PE depolymerization

| Reaction | | | | | | | | | | |
|---|---|---|---|---|---|---|---|---|---|---|
| conditions ^{a} | | | | | | | | m_{benz}/ | mₐᵣₒₘ/ | m_{benz}/ |
| First | Second | m_{gas} ^{c} | m_{liquid} ^{d} | m_{wax} ^{e} | n_{c-c scission. corr} ^{f} | | n_{benz} ^{h} | mₐᵣₒₘ ⁱ | mₜₒₜₐₗ ^{j} | m_{PE} ^{k} |
| stage ^{b} | stage ^{b} | (wt %) | (wt %) | (wt %) | (mmol) | [symbol] | (μmol) | (wt %) | (wt %) | (wt %) |
| Ar | Ar | 2 | 8 | 46 | 0.15 | 61 | 12 | 29 | 41 | 1 |
| 2 bar | 2 bar | | | | | | | | | |
| H₂ | H₂ | 14 | 44 | 30 | 0.96 | 21 | <1 | — | <1 | <1 |
| 8 bar | 8 bar | | | | | | | | | |
| Ar | H₂ | 2 | 16 | 41 | 0.21 | 40 | 20 | 77 | 13 | 2 |
| 2 bar | 8 bar | | | | | | | | | |
| H₂ | Ar | 5 | 39 | 38 | 0.58 | 24 | 27 | 50 | 13 | 3 |
| 8 bar | 2 bar | | | | | | | | | |

^{a} Reaction conditions: PE (0.200 g, M_{w} = 3.5 × 10³ g mol⁻¹, Ð = 1.9), Pt/SiO₂-Al₂O₃ (0.100 g, 1.7 wt % Pt), 275 °C, 10 mL, stirred Parr reactor.
^{b} Pressure measured at room temperature. The duration of each reaction stage was 4 h.
^{c} C₁-C₆, as measured by GC-FID.
^{d} C₇-C₃₀, as measured by GC-FID.
^{e} C_{>30}, as measured by weight.
^{f} Total number of C—C bond scission events, calculated as N(t)-N(0), where N(0) and N(t) represent the total number of chains present at time 0 and time t, respectively. Any missing mass was assumed to have the same distribution as the recovered mass (see Section: Analysis of Carbon-Carbon Bond Scission).
^{g} Average carbon number in the combined liquid and wax products.
^{h} Yields of alkylbenzenes (n_{benz}) in liquids, estimated by combining the integrated ¹H NMR intensity and the GC-FID product distribution (see Section: Selectivity and Yield of Aromatics).
ⁱ Mass selectivity of alkylbenzene relative to total aromatics.
^{j} Mass selectivity of alkylaromatics relative to total hydrocarbons (mₜₒₜₐₗ) in liquids, where mₜₒₜₐₗ = mₐₗₖ + m_{benz} + mₙₐₚₕ + mₚₕₑₙ.
^{k} Mass yield of alkylbenzene relative to the initial mass of PE.

Conducting the first reaction stage under Ar, followed by a second stage under 8 bar H₂, yielded liquid/wax with an intermediate average carbon number of 39, consistent with the shorter time in the fast hydrogenolysis conditions. However, the yield of alkylbenzenes, 20 μmol, doubled relative to the yield under Ar alone. An even better result was obtained when the first reaction stage was conducted under 8 bar H₂, followed by a second stage under Ar. For this experiment, the average carbon number was lower (24) and the alkylbenzene yield was even higher (27 μmol). Presumably the initial reduction in molecular weight under H₂ without polyaromatic formation reduced catalyst poisoning while accelerating the subsequent aromatization of the lower molecular weight alkanes.

The effectiveness of this strategy for alkylbenzene production was tested with a higher PE: catalyst ratio (0.400 g PE, M_{w}=3.5×10³ g mol⁻¹, Ð=1.9 and 0.100 g Pt/SiO₂—Al₂O₃) in a larger-volume reactor (100 mL) at 275 °C. The reaction was conducted initially under 4 bar H₂ for 4 h, followed by 4 h under 2 bar Ar. The alkylbenzene yield was 0.21 g (g_{catalyst})⁻¹ with an average carbon number C₁₇ (5 wt % relative to initial charge of PE). This yield is 3 times higher than that reported previously in a process catalyzed by Pt/F—Al₂O₃ in generating C₂₃. Furthermore, the alkylbenzene selectivity was 56 mol % relative to all aromatics, compared to 34 mol % in the earlier process.

### CONCLUSION

P(H₂) is a determining parameter in the bifunctional catalytic depolymerization of polyethylene to alkylaromatics. H₂ was needed for C—C bond scission, reducing the molecular weight of hydrocarbons through hydrocracking, while also affecting the selectivity to alkylbenzenes relative to polyaromatics. Consistent with thermodynamic predictions, the highest yields of alkylbenzenes were obtained at moderate P(H₂) values, 3-5 bar, while the highest selectivities for alkylbenzenes relative to polyaromatics were achieved under 6-7 bar P(H₂).

The rate of C—C bond scission was pseudo-zeroth-order with respect to the number of alkane chains. The order with respect to H₂ was positive at low pressures and negative at high pressure. The non-monotonic behavior is consistent with a traditional hydrocracking mechanism combined with competitive adsorption of aromatics. The formation of aromatics was pseudo-first-order and the total aromatic yield decreased with increasing P(H₂). The formation of polyaromatics at low P(H₂) deactivated the catalyst. These findings can guide catalyst design and selection of reaction conditions for selective conversion of polyethylene to surfactant-range alkylaromatics.

### REFERENCES

Johnson, J. V., et al., Int. J. Mass Spectrom. Ion Process. 1991, 106,
Khmel'nitskii, R. A., et al., Chem. Technol. Fuels Oils 1967, 3 (2), 140-143.
McClennen, W. H., et al., Combust. Sci. Technol. 1990, 74 (1-6), 297-309.
Chen, J.; Zhang, H., et al., Org. Geochem. 2013, 61, 6-14.
Marinho, R. S., et al., J. Hazard. Mater. 2011, 192 (3), 1155-1160.
Sun, J., et al., Chem. 2023, 9 (8), 2318-2336.
Lee, Y.-H., et al., STAR Protoc. 2023, 4 (4), 102575.
Itakura, M., et al., J. Appl. Polym. Sci. 2004, 94 (3), 1101-1106.
Chen, Z., et al., Chem. Eng. J. 2022, 136213.
Zhang, F., et al., Science 2020, 370 (6515), 437-441.
Madhushaw, R. J., et al., J. Am. Chem. Soc. 2004, 126 (47), 15560-15565.
Watson, M. D., et al., Chem. Rev. 2001, 101 (5), 1267-1300.
Chanyshev, A. D., et al., Sci. Rep. 2017, 7 (1), 7889.
Weitkamp, J., ChemCatChem 2012, 4 (3), 292-306.
Pennel, M. L., et al., ACS Sustain. Chem. Eng. 2023, 11 (34), 12623-12630.
Serrano, D. P., et al., ACS Catal. 2012, 2 (9), 1924-1941.
Celik, G., et al., ACS Cent. Sci. 2019, 5 (11), 1795-1803.
Yappert, R., et al., J. Mater. Chem. A 2022, 10 (45), 24084-24095.
Smith, G. C., Catalytic Cracking of n-Alkanes and n-Alkylbenzenes over H-ZSM-5 Zeolite, Massachusetts Institute of Technology, 1993.
le Goff, P.-Y., et al., Catalytic Reforming, In Springer Handbook of Petroleum Technology;
Hsu, C. S., Robinson, P. R., Eds.; Springer International Publishing: Cham, 2017; pp 589-616.
Ali, S. A., Pet. Sci. Technol. 2007, 25 (10), 1293-1304.
Liu, S., et al., Sci. Adv. 2021, 7 (17), eabf8283.
Qiu, Z., et al., Sci. Adv. 2023, 9 (25), eadg5332.
Rossetti, I., et al., Chem. Eng. J. 2009, 154 (1), 295-301.
Ribeiro, F., et al., J. Catal. 1982, 78 (2), 267-274.
Snyder, L. R., J. Phys. Chem. 1963, 67 (2), 234-240.

1. A process for upcycling a waste material, wherein the waste material comprises a hydrocarbon polymer, comprising
(i) feeding the waste material into a reactor,
wherein the reactor comprises a catalyst therein,
wherein the catalyst comprises a transition metal; and
(ii) operating the reactor under a sufficient hydrogen gas pressure (P(H₂)) to form a product comprising a liquid comprising one or more alkylbenzene compound(s).

2. The process of claim 1, wherein the liquid further comprises one or more other aromatic compounds and optionally one or more alkane and/or one or more non-aromatic compounds, and wherein the mass selectivity of the one or more alkylbenzene compound(s) relative to all aromatic compounds in the liquid is at least 25%, at least 30%, at least 50%, in a range from 25% to 99.99%, from 25% to 99.9%, from 25% to 99%, from 25% to 95%, from 25% to 90%, from 25% to 90%, from 25% to 80%, from 25% to 70%, or from 25% to 60%.

3. (canceled)

4. (canceled)

5. The process of claim 2, wherein:
(a) the number of carbon atoms in each of the one or more alkylbenzene compound(s) ranges from 7 to 40 carbon atoms or from 10 to 30 carbon atoms, and/or
(b) each of the one or more alkylbenzene compound(s) has a weight average molecular weight (Mw) in a range from 150 g mol⁻¹ to 800 g mol⁻¹, from 200 g mol⁻¹ to 800 g mol⁻¹, from 200 g mol⁻¹ to 600 g mol⁻¹, from 200 g mol⁻¹ to 500 g mol⁻¹, or from 200 g mol⁻¹ to 400 g mol⁻¹.

6. (canceled)

7. The process of claim 2, wherein each of the one or more alkylbenezene compound(s) has a structure of Formula (I): 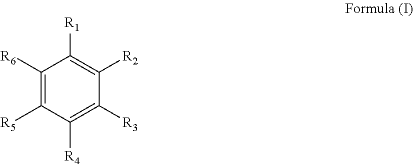
wherein R₁-R₆ are independently hydrogen or an alkyl group, and wherein at least one of R₁-R₆ is an alkyl group, optionally wherein the alkyl group is —CH₂R, and R is a hydrogen, C₁-C₁₉ alkyl group, C₁-C₁₅ alkyl group, a C₁-C₁₂ alkyl group, a C₁-C₁₀ alkyl group, or a C₁-C₅ alkyl group.

8. (canceled)

9. (canceled)

10. The process of claim 2, wherein the transition metal is selected from the group consisting of platinum, palladium, ruthenium, iridium, rhenium, rhodium, iron, cobalt, nickel, copper, molybdenum, and tungsten.

11. The process of claim 2, wherein the catalyst comprises more than one transition metal, wherein each of the transition metals is independently selected from the group consisting of platinum, palladium, ruthenium, iridium, rhenium, rhodium, iron, cobalt, nickel, copper, molybdenum, and tungsten,
optionally wherein the catalyst further comprises a second metal, wherein the second metal is different from each of the transition metals, and wherein the second metal is selected from the group consisting of rhenium, tin, lead, tungsten, molybdenum, chromium, manganese, and zinc, or a combination thereof.

12. (canceled)

13. (canceled)

14. The process of claim 2, wherein the catalyst is dispersed on a surface of a first support and wherein the catalyst is in the form of atoms, nanoclusters, or nanoparticles, or a combination thereof,
optionally wherein the total weight loading of the transition metal is in a range from 0.1 wt % to 10 wt %, from 0.1 wt % to 8 wt %, from 0.1 wt % to 5 wt %, from 0.1 wt % to 1 wt %, from 0.5 wt % to 10 wt %, from 0.5 wt % to 8 wt %, from 0.5 wt % to 5 wt %, from 1 wt % to 10 wt %, from 1 wt % to 8 wt %, or from 1 wt % to 5 wt % of the total weight of the catalyst and the first support.

15. The process of claim 14, wherein the catalyst is platinum nanoparticles, wherein the first support is an aluminum oxide support or silica-aluminum oxide support.

16. The process of claim 14, further comprising a second support, wherein the first support is combined with the second support and the second support is an acidic support, optionally wherein:
(a) the total weight loading of the transition metal is in a range from 0.1 wt % to 10 wt %, from 0.1 wt % to 8 wt %, from 0.1 wt % to 5 wt %, from 0.1 wt % to 1 wt %, from 0.5 wt % to 10 wt %, from 0.5 wt % to 8 wt %, from 0.5 wt % to 5 wt %, from 1 wt % to 10 wt %, from 1 wt % to 8 wt %, or from 1 wt % to 5 wt % of the total weight of the catalyst and the first and second support, and/or
(b) the weight percentage of the second support is in a range from 2% to 90%, from 2% to 50%, from 2% to 35%, from 10% to 90%, from 10% to 80%, from 10% to 70%, from 10% to 60%, from 10% to 50%, from 10% to 30%, from 15% to 40%, from 20% to 50%, from 20% to 40%, or from 20% to 35% of the total weight of the catalyst and the first and second support.

17. The process of claim 16, wherein the second support is a halogenated alumina support, optionally a fluorinated alumina support, and the weight percentage of the halogen, optionally the fluorine, in the second support is in a range from 0.1% to 5%, from 0.5% to 3%, from 0.5% to 2%, from 0.5% to 1.5%, optionally about 0.7% or about 1.3%.

18. (canceled)

19. (canceled)

20. (canceled)

21. The process of claim 2, further comprising;
step (a) feeding hydrogen gas into the reactor during step (i) or after step (i) and prior to step (ii), and optionally purifying the hydrogen gas prior to step (a);
step (b) feeding hydrogen gas into the reactor during step (ii) to maintain the P(H₂), wherein step (b) occurs one or more times;
(iii) processing the waste prior to step (i), wherein the waste is in the form of a solid, and wherein step (iii) includes shredding, cutting, and/or grinding the waste hydrocarbon polymer to small parts, or dissolving the solid waste in a solvent, optionally in a hydrocarbon solvent; and/or
step (e) separating the liquid and a wax in the product.

22. (canceled)

23. (canceled)

24. The process of claim 2, wherein during step (ii), the sufficient P(H₂) in the reactor is maintained at a value in a range from 1 atm to 8 atm, from 1 atm to 7 atm, from 1 atm to 6 atm, from 1 atm to 5 atm, or from 1 atm to 4 atm.

25. The process of claim 1, further comprising, after step (i) and before step (ii),
(iia) operating the reactor under a first P(H₂) to form fragments of the hydrocarbon polymer,
wherein the sufficient P(H₂) is lower than the first P(H₂); or
(iia) operating the reactor under a first P(H₂) to form fragments of the hydrocarbon polymer, and
after step (iia),
(iib) operating the reactor at a second P(H₂), to form aromatic compounds,
wherein the second P(H₂) is lower than the first P(H₂), and the sufficient P(H₂) is higher than the second P(H₂).

26. The process of claim 25, wherein:
step (ii) further comprises removing hydrogen gas from the reactor and, optionally, feeding an inert gas into the reactor to provide the sufficient P(H₂), and optionally, an inert gas pressure, in the reactor; or
step (iib) further comprises removing hydrogen gas from the reactor and, optionally, feeding an inert gas into the reactor to provide the second P(H₂), and optionally, an inert gas pressure, in the reactor.

27. The process of claim 25, wherein the first P(H₂) ranges from 5 atm to 20 atm, from 5 atm to 18 atm, from 5 atm to 15 atm, from 5 atm to 12 atm, from 5 atm to 10 atm, such as about 8 atm, and/or the sufficient P(H₂) or second P(H₂) ranges from 0 to <5 atm, from 0 to 4 atm, from 0 to 3 atm, or from 0 to 2 atm.

28. The process of claim 26, wherein;
in step (ii), the sufficient P(H₂) is 0 and the reactor is under the inert gas pressure ranging from 0 to <5 atm, from 0 to 4 atm, from 0 to 3 atm, or from 0 to 2 atm; or
in step (iib), the second P(H₂) is 0 and the reactor is under the inert gas pressure ranging from 0 to <5 atm, from 0 to 4 atm, from 0 to 3 atm, or from 0 to 2 atm.

29.-33. (canceled)

34. The process of claim 25, wherein step ii further comprises feeding hydrogen gas into the reactor to provide the sufficient P(H₂) in the reactor, optionally wherein the sufficient P(H₂) ranges from 5 atm to 15 atm, from 5 atm to 12 atm, or from 5 atm to 10 atm.

35. (canceled)

36. The process of claim 25, further comprising:
step (c) measuring the fragments of the hydrocarbon polymers during step (iia) to determine the weight average molecular weight (Mw) of the fragments, wherein step (c) occurs one or more times,
step (d) measuring the aromatic compounds during step (ii) to determine the total yield of the aromatic compounds, wherein step (d) occurs one or more times; and/or
step (a) feeding hydrogen gas into the reactor during step (i) or after step (i) and prior to step (iia), and optionally purifying the hydrogen gas prior to step (a).

37.-61. (canceled)

62. A composition comprising one or more alkylbenzene compound(s), wherein the composition is in the form of a liquid, and wherein the composition is produced by the process of claim 2.

63. A composition comprising an alkylbenzene compound having the structure of: 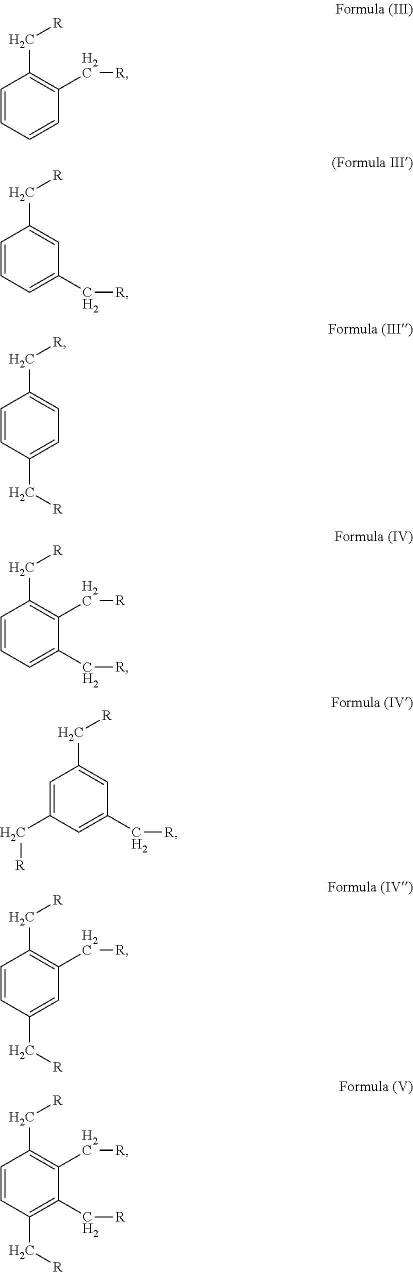 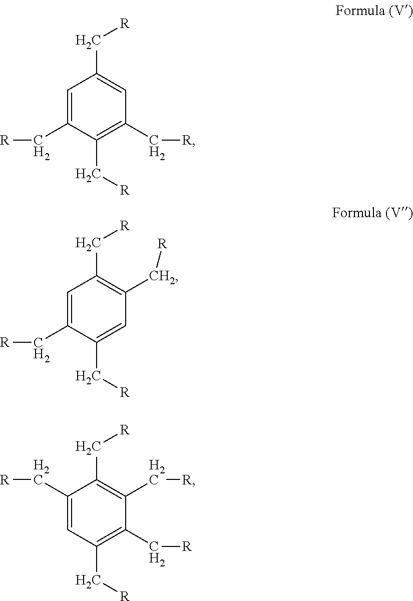
Formula (VI)
wherein two R groups are independently a linear C₃-C₁₅ alkyl group, a linear C₃-C₁₂ alkyl group, a linear C₃-C₁₀ alkyl group, a linear C₃-C₈ alkyl group, a linear C₄-C₁₅ alkyl group, a linear C₄-C₁₂ alkyl group, a linear C₄-C₁₀ alkyl group, or a linear C₄-C₈ alkyl group, such as a linear C₄ or a linear C₅ alkyl group; and the other R group(s) are hydrogen.

64.-67. (canceled)
